SEQUENCE LISTING (25 sequences)

SEQ ID NO: 1 - protein, 200 aa, Homo sapiens
MSMLFYTLITAFLIGIQAEPHSESNVPAGHTIPQVHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSY

SEQ ID NO: 2 - DNA, 726 bp, Homo sapiens
atgtccatgttgttctacactctgatcacagcttttctgatcggcatacaggcggaaccacactcagagagcaatgtccctgcaggacacaccatcccccaagtccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagcctga

SEQ ID NO: 3 - protein, 211 aa, Homo sapiens
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 4 - DNA, 636 bp, Homo sapiens
atgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctga

SEQ ID NO: 5 - DNA, 615 bp, Artificial Sequence
  CAG Promoter
ataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactg

SEQ ID NO: 6 - protein, 735 aa, Dependoparvovirus adeno-associated dependoparvovirus A [sequence omitted]

SEQ ID NO: 7 - DNA, 2208 bp, Dependoparvovirus adeno-associated dependoparvovirus A
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 8 - protein, 533 aa, Dependoparvovirus adeno-associated dependoparvovirus A
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 9 - DNA, 1602 bp, Dependoparvovirus adeno-associated dependoparvovirus A
atggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 10 - protein, 724 aa, Dependoparvovirus adeno-associated dependoparvovirus A
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 11 - DNA, 2175 bp, Dependoparvovirus adeno-associated dependoparvovirus A
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 12 - protein, 738 aa, Dependoparvovirus adeno-associated dependoparvovirus A
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 13 - DNA, 2214 bp, Dependoparvovirus adeno-associated dependoparvovirus A
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 14 - protein, 736 aa, Dependoparvovirus adeno-associated dependoparvovirus A
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 15 - DNA, 2209 bp, Dependoparvovirus adeno-associated dependoparvovirus A
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgt

SEQ ID NO: 16 - DNA, 613 bp, Artificial Sequence
  CMV Promoter
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaat

SEQ ID NO: 17 - DNA, 726 bp, Artificial Sequence
  Codon optimized NGF
atgagcatgctgttttacaccctgatcaccgccttcctgatcggcatccaggccgagccccacagcgagagcaacgtgcccgctggacacacaatccctcaggcccactggacaaaactgcagcatagcctggatacagccctgagaagggccagaagcgcccctgccgccgccatcgccgctagagtggccggccagacaagaaatatcaccgtggacccccggctgttcaagaaaagaagactgcggtctcctagagtgctcttttcaacacagcctcctcgggaagccgctgatacccaggacctggacttcgaggtgggaggcgccgctcctttcaatagaacccacagatccaagagaagctctagccaccctatcttccaccggggcgagttcagcgtgtgcgacagcgtttctgtgtgggtgggagataagaccaccgcaaccgatatcaagggcaaggaagtgatggtgctgggcgaagtgaacatcaacaacagcgtctttaagcagtacttcttcgagacaaagtgccgggacccaaaccccgtggacagcggctgcagaggcattgactccaagcactggaactcctactgcaccacaacacacaccttcgtgaaggccctgaccatggacggcaaacaagctgcctggcggttcatcagaatcgacaccgcctgtgtctgtgtgctgagcagaaaggccgtgcgccgggcctag

SEQ ID NO: 18 - DNA, 635 bp, Artificial Sequence
  Codon Optimized GDNF
atgaactgtgggacgttgtggccgtgtgcctggtcctcctgcacaccgcctccgcctttccactgcctgctggaaagcggcctcctgaggcccctgctgaagatagaagcctgggcagaagaagagcccccttcgccctgagctctgatagcaacatgcctgaggactaccccgaccagttcgacgacgtgatggactttatccaggccaccatcaagagactgaaacggagccctgacaagcagatggccgtgctgcctagacgggaaagaaatagacaggccgcagctgccaaccccgagaacagcagaggcaaaggccggagaggccagaggggaaaaaacagaggatgtgtgctgaccgccatccatctgaacgtgacagatctgggcctgggctatgagaccaaggaagagctgatcttcagatactgcagcggcagctgcgacgccgccgagacaacctacgacaagatcctgaagaacctgtcccggaatcggcggctggtgtctgacaaggtgggccaagcttgctgcagaccaattgccttcgacgatgatctgtctttcctggatgacaacctggtgtaccacatcctgagaaagcacagcgccaagcgctgcggctgtatctag

SEQ ID NO: 19 - DNA, 145 bp, Artificial Sequence
  5′ ITR Sequence
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaac

SEQ ID NO: 20 - DNA, 145 bp, Artificial Sequence
  3′ ITR Sequence
gttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 21 - DNA, 228 bp, Artificial Sequence
  bGHpolyA Sequence
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 22 - DNA, 411 bp, Artificial Sequence
  WPRE Sequence
tcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctccctatcgccacggcagaactcatcgccgcctgccttgcccgctgctggacaggggctaggttgctgggcactgataattccgtggtgttgtcggggaagctgacgtc

SEQ ID NO: 23 - DNA, 2967 bp, Artificial Sequence
  Expression cassette comprising sequence encoding GDNF
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacggatctttacaaattcaagccaggtgatttcaacaaattttgctgacgatttaggcgcactatcccctaaactacaaattagaaaatagcgttccttgacactagtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaataagctttctcaggggagatctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacgccgccgccatgaagctgtgggacgttgtggccgtgtgcctggtcctcctgcacaccgcctccgcctttccactgcctgctggaaagcggcctcctgaggcccctgctgaagatagaagcctgggcagaagaagagcccccttcgccctgagctctgatagcaacatgcctgaggactaccccgaccagttcgacgacgtgatggactttatccaggccaccatcaagagactgaaacggagccctgacaagcagatggccgtgctgcctagacgggaaagaaatagacaggccgcagctgccaaccccgagaacagcagaggcaaaggccggagaggccagaggggaaaaaacagaggatgtgtgctgaccgccatccatctgaacgtgacagatctgggcctgggctatgagaccaaggaagagctgatcttcagatactgcagcggcagctgcgacgccgccgagacaacctacgacaagatcctgaagaacctgtcccggaatcggcggctggtgtctgacaaggtgggccaagcttgctgcagaccaattgccttcgacgatgatctgtctttcctggatgacaacctggtgtaccacatcctgagaaagcacagcgccaagcgctgcggctgtatctagtcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctccctatcgccacggcagaactcatcgccgcctgccttgcccgctgctggacaggggctaggttgctgggcactgataattccgtggtgttgtcggggaagctgacgtccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagcgctggctagaattacctaccggcctccaccataccttcgatattcgcgcccactctcccattaatccgcacaagtggatgtgatgcgattgcccgctaagatagttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 24 - DNA, 3054 bp, Artificial Sequence
  Expression cassette comprising sequence encoding NGF
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacggatctttacaaattcaagccaggtgatttcaacaaattttgctgacgatttaggcgcactatcccctaaactacaaattagaaaatagcgttccttgacactagtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaataagctttctcaggggagatctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacgccgccgccatgagcatgctgttttacaccctgatcaccgccttcctgatcggcatccaggccgagccccacagcgagagcaacgtgcccgctggacacacaatccctcaggcccactggacaaaactgcagcatagcctggatacagccctgagaagggccagaagcgcccctgccgccgccatcgccgctagagtggccggccagacaagaaatatcaccgtggacccccggctgttcaagaaaagaagactgcggtctcctagagtgctcttttcaacacagcctcctcgggaagccgctgatacccaggacctggacttcgaggtgggaggcgccgctcctttcaatagaacccacagatccaagagaagctctagccaccctatcttccaccggggcgagttcagcgtgtgcgacagcgtttctgtgtgggtgggagataagaccaccgcaaccgatatcaagggcaaggaagtgatggtgctgggcgaagtgaacatcaacaacagcgtctttaagcagtacttcttcgagacaaagtgccgggacccaaaccccgtggacagcggctgcagaggcattgactccaagcactggaactcctactgcaccacaacacacaccttcgtgaaggccctgaccatggacggcaaacaagctgcctggcggttcatcagaatcgacaccgcctgtgtctgtgtgctgagcagaaaggccgtgcgccgggcctagtcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctccctatcgccacggcagaactcatcgccgcctgccttgcccgctgctggacaggggctaggttgctgggcactgataattccgtggtgttgtcggggaagctgacgtccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagcgctggctagaattacctaccggcctccaccataccttcgatattcgcgcccactctcccattaatccgcacaagtggatgtgatgcgattgcccgctaagatagttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcg

SEQ ID NO: 25 - DNA, 5895 bp, Artificial Sequence
  pAAV-CMVE-NGF
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggattgacctaacgaaaagtgaacttcataatacgtgctgtcccacgcacatggtagatttggacaaaattgaatggagtctgatcaaccttcacaccgatctagatatcgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacggatctttacaaattcaagccaggtgatttcaacaaattttgctgacgatttaggcgcactatcccctaaactacaaattagaaaatagcgttccttgacactagtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaataagctttctcaggggagatctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacgccgccgccatgagcatgctgttttacaccctgatcaccgccttcctgatcggcatccaggccgagccccacagcgagagcaacgtgcccgctggacacacaatccctcaggcccactggacaaaactgcagcatagcctggatacagccctgagaagggccagaagcgcccctgccgccgccatcgccgctagagtggccggccagacaagaaatatcaccgtggacccccggctgttcaagaaaagaagactgcggtctcctagagtgctcttttcaacacagcctcctcgggaagccgctgatacccaggacctggacttcgaggtgggaggcgccgctcctttcaatagaacccacagatccaagagaagctctagccaccctatcttccaccggggcgagttcagcgtgtgcgacagcgtttctgtgtgggtgggagataagaccaccgcaaccgatatcaagggcaaggaagtgatggtgctgggcgaagtgaacatcaacaacagcgtctttaagcagtacttcttcgagacaaagtgccgggacccaaaccccgtggacagcggctgcagaggcattgactccaagcactggaactcctactgcaccacaacacacaccttcgtgaaggccctgaccatggacggcaaacaagctgcctggcggttcatcagaatcgacaccgcctgtgtctgtgtgctgagcagaaaggccgtgcgccgggcctagtcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctccctatcgccacggcagaactcatcgccgcctgccttgcccgctgctggacaggggctaggttgctgggcactgataattccgtggtgttgtcggggaagctgacgtccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagcgctggctagaattacctaccggcctccaccataccttcgatattcgcgcccactctcccattaatccgcacaagtggatgtgatgcgattgcccgctaagatagttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcgatatcgaatcgaatgcagtgatctcccaggtgcgaatcaaaaatactaggtaactagaggatttgcgacgttctaagcgttggtccatgtgaatcgccatccaggatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

(19) United States
(12) Patent Application Publication
(10) Pub. No.: US 2024/0238375 A1
(43) Pub. Date: Jul. 18, 2024

(54) AAV VIRION ENCODING NEUROTROPHIC FACTOR AND USES THEREOF

(71) Applicant: OYSTER POINT PHARMA, INC., Princeton, NJ (US)

(72) Inventors: Jeffrey Alan NAU, Princeton, NJ (US); Eric C. CARLSON, Princeton, NJ (US)

(21) Appl. No.: 18/559,512

(22) PCT Filed: May 4, 2022
(86) PCT No.: PCT/US2022/027647

§ 371 Date: Nov. 7, 2023

Related U.S. Application Data
(60) Provisional application No. 63/185,889, filed on May 7, 2021.

(51) Int. Cl. A61K 38/18; A61K 9/00; A61K 48/00; A61P 27/02; C12N 15/86

(52) U.S. Cl. CPC A61K 38/185; A61K 9/0048; A61K 48/0033; A61K 48/0066; A61P 27/02; C12N 15/86; C12N 2750/14122; C12N 2750/14143; C12N 2750/14171

(57) ABSTRACT

Provided are recombinant adeno-associated virus (rAAV) virions, methods of treating an ocular condition, pharmaceutical compositions, and other compositions and methods, in which the rAAV virion comprises a poly nucleotide encoding a neurotrophic factor, such as Nerve Growth Factor (NGF) or Glial Derived Neurotrophic Factor (GDNF). Methods of treatment may include administration to the lacrimal gland.

### CROSS-RELATED APPLICATIONS

This application claims the benefit of U.S. Provisional Patent Application Ser. No. 63/185,889 filed May 7, 2021. The entire contents of which is incorporated herein by this reference.

### REFERENCE TO SEQUENCE LISTING

The instant application contains a Sequence Listing which has been submitted electronically in ASCII format via EFS-Web, and is hereby incorporated by reference in its entirety. Said ASCII copy, created on Apr. 29, 2022, is named OYST_015_03WO_SeqList_ST25 and is 72,019 bytes in size.

### BACKGROUND

Medical treatment of ocular conditions that affect the ocular surface, such as neurotrophic keratopathy; chemical burn; corneal wounds; persistent epithelial defect; dry eye disease; herpes simplex viral infection of the trigeminal nerve and/or the eye; varicella zoster virus infection of the trigeminal nerve and/or the eye; and diabetic complications of the corneal nerves, are known. In addition, medical treatment of glaucoma, presbyopia, myopia, and other anterior chamber disorders are known. These medical treatments include topical eye drops, oral pharmaceuticals, and others.

Despite available treatments these ocular conditions remain a challenge for ophthalmologists and therapeutic strategies focus on the severity of the disease. Treatments for less severe cases may include bandage contact lenses and lubricating eyedrops. For more severe cases, amniotic membrane transplantation, blood derivates at specified concentrations in serum tears, tarsorrhaphy, and topically applied autologous serum eye drops have been widely used (Di Zazzo et al. Ocul Surf. 17 (4):619-623 (2019)). Emerging treatment strategies are derived from the efficacy of autologous serum eye drops. Autologous serum eye drops are prepared by diluting serum harvested from patient blood with saline solution ((Bradley et al. Clin Exp Ophthalmol. 36 (8):717-20 (2008)). The preparation is performed on an individual basis, and is thought to be effective in treating ocular conditions due to the presence of growth factors, including neurotrophic factors such as NGF and glial cell line-derived neurotrophic factor (GDNF).

Delivery of neurotrophic factors for treatment of the ocular surface currently relies on repeated application of autologous serum eye drops or eye drops containing a purified recombinant neurotrophic factor. Autologous serum eye drops may vary due to differences in the serum composition between patients, the possibility for contamination during preparation of the drops, and special storage conditions required to minimize degradation of the neurotrophic factors. Likewise, eye drops containing recombinant neurotrophic factors require expression and purification conditions that result in an active neurotrophic factor and have limited stability and require special storage conditions. In addition, recombinant human neurotrophic factor, such as NGF, must be administered by the patient 6 times per day at 2-hour intervals, for eight weeks to provide benefit. Thus, there is a need for methods to deliver neurotrophic factors to the ocular surface without the need for repeated topical applications that can be challenging or impossible for some patients. Improvement to medical treatment of ocular surface disorders would be desirable.

### SUMMARY OF INVENTION

The disclosure relates generally to expression of neurotrophic factors in the eye using adeno-associated virus (AAV) vectors. For example, the disclosure provides vectors for expression of Nerve Growth Factor (NGF) or Glial Derived Neurotrophic Factor (GDNF), and medical uses thereof.

In one aspect, the disclosure provides a method of treating an ocular condition in a subject in need thereof, the method comprising administering an rAAV virion, the rAAV virion comprising an AAV capsid and an expression cassette comprising a polynucleotide encoding a neurotrophic factor operatively linked to a promoter, to the eye of the subject or to a lacrimal gland of the eye of the subject. In some embodiments, the rAAV virion is administered to at least one eye and/or lacrimal gland of the subject and transduces the cells of the at least one eye and/or lacrimal gland of a subject. Transduced cells within at least one eye and/or lacrimal gland express a neurotrophic factor. The neurotrophic factor expressed by the transduced cells of the at least one eye and/or lacrimal gland express the neurotrophic factor into the tear film and/or onto the ocular surface of the subject. The expressed neurotrophic factor can be, for example, human Nerve Growth Factor (NGF) protein or Glial Derived Neurotrophic Factor (GDNF) protein. Expression of the gene product results in a reduction of the symptoms associated with the ocular condition in the subject.

In some embodiments, the disclosure provides a method of treating an ocular condition in a subject in need thereof, the method comprising administering a recombinant adeno-associated virus (rAAV) virion, the rAAV virion comprising an AAV capsid and an expression cassette comprising a polynucleotide encoding a neurotrophic factor operatively linked to a promoter, wherein the rAAV virion is administered to at least one eye of the subject or to at least one lacrimal gland of the eye of the subject, thereby treating an ocular condition in a subject in need thereof.

In some embodiments, the rAAV virion is administered to at least one lacrimal gland of the subject. In some embodiments, the lacrimal gland is the main lacrimal gland. In some embodiments, the at least one lacrimal gland is the main lacrimal gland or any one of the Wolfring's glands or the Krause's glands of the subject. In some embodiments, cells within the at least one lacrimal gland are transduced by the rAAV virion. In some embodiments the transduced cells within the at least one lacrimal gland express a therapeutically effective amount of the neurotrophic factor into the tear film and optionally onto the ocular surface of the subject. In some embodiments the transduced cells within the at least one lacrimal gland express an effective amount of the neurotrophic factor into the tear film and optionally onto the ocular surface of the subject.

In some embodiments, the neurotrophic factor is a Nerve Growth Factor (NGF) protein. In some embodiments, the polynucleotide encoding the NGF protein comprises a sequence that shares at least 95% identity to (SEQ ID NO. 2) In some embodiments, the NGF protein comprises a sequence that shares at least 95% identity to (SEQ ID NO: 1). In some embodiments, the NGF protein comprises SEQ ID NO: 1. In some embodiments, the polynucleotide encoding the NGF protein comprises SEQ ID NO: 2.

In some embodiments, the neurotrophic factor is a Glial Derived Neurotrophic Factor (GDNF) protein. In some embodiments, the polynucleotide encoding the GDNF protein comprises a sequence that shares at least 95% identity to (SEQ ID NO: 4). In some embodiments, the GDNF protein comprises a sequence that shares at least 95% identity to (SEQ ID NO: 3). In some embodiments, the GDNF protein comprises SEQ ID NO: 3. In some embodiments, the polynucleotide encoding the GDNF protein comprises SEQ ID NO: 4.

In some embodiments, the AAV capsid comprises a capsid that shares at least 95%, 98%, or 100% identity to a capsid of AAV2 VP1, AAV2 VP3, AAV5, AAV8, or AAV9.

In some embodiments, the promoter is a CMV enhancer, chicken beta-Actin (CAG) promoter (SEQ ID NO: 5).

In some embodiments, the ocular condition is chemical burn of the ocular surface, corneal wound, persistent epithelial defect, dry eye disease, neurotrophic keratitis, herpes simplex viral infection of the trigeminal nerve and/or the eye, varicella zoster virus infection of the trigeminal nerve, and/or the eye, and diabetic complications of the corneal nerves.

In some embodiments, the ocular condition is chemical burn of the ocular surface, corneal wound, corneal ulcer, persistent epithelial defect, dry eye disease, neurotrophic keratitis, herpes simplex viral infection of the trigeminal nerve and/or the eye, varicella zoster virus infection of the trigeminal nerve, and/or the eye, and diabetic complications of the corneal nerves.

In some embodiments, the ocular condition is characterized by clinical signs as defined under Mackie's Classification.

In some embodiments, one or more symptoms of the ocular condition are reduced compared to the symptoms of the ocular condition before administration of the rAAV virion. In some embodiments, one or more symptoms of the ocular condition are reduced compared to the symptoms of the ocular condition in an untreated control subject. In some embodiments, one or more symptoms of the ocular condition are reduced compared to the symptoms of the ocular condition in a contralateral eye.

In another aspect, the disclosure provides an rAAV virion, comprising an AAV capsid and an expression cassette comprising a polynucleotide encoding a neurotrophic factor operatively linked to a promoter.

In some embodiments, the neurotrophic factor is a Nerve Growth Factor (NGF) protein. In some embodiments, the polynucleotide encoding the NGF protein comprises a sequence that shares at least 95% identity to (SEQ ID NO: 2). In some embodiments, the NGF protein comprises a sequence that shares at least 95% identity to (SEQ ID NO: 1). In some embodiments, the NGF protein comprises SEQ ID NO: 1. In some embodiments, the polynucleotide encoding the NGF protein comprises SEQ ID NO: 2.

In some embodiments, the neurotrophic factor is a Glial Derived Neurotrophic Factor (GDNF) protein. In some embodiments, the polynucleotide encoding the GDNF protein comprises a sequence that shares at least 95% identity to (SEQ ID NO: 4). In some embodiments, the GDNF protein comprises a sequence that shares at least 95% identity to (SEQ ID NO: 3). In some embodiments, the GDNF protein comprises SEQ ID NO: 3. In some embodiments, the polynucleotide encoding the GDNF protein comprises SEQ ID NO: 4.

In some embodiments, the AAV capsid comprises a capsid that shares at least 95%, 98%, or 100% identity to a capsid of AAV2 VP1, AAV2 VP3, AAV5, AAV8, or AAV9.

In some embodiments, the promoter is a CAG promoter (SEQ ID NO: 5).

In one aspect, the disclosure relates to a pharmaceutical composition comprising the rAAV virion as disclosed herein and a pharmaceutically acceptable carrier. In some embodiments, the pharmaceutical composition comprises about 1×10⁹ to about 1×10¹⁴ genome copies per milliliter of the rAAV virion. In some embodiments, the pharmaceutical composition comprises about 1×10⁷ to about 1×10¹⁴ genome copies per milliliter of the rAAV virion. In some embodiments, the pharmaceutical composition comprises about 1×10¹² to about 6.2×10¹² genome copies per milliliter of the rAAV virion.

In one aspect, the disclosure relates to a method of treating an ocular condition in a subject in need thereof, the method comprising administering a therapeutically effective amount of the pharmaceutical composition as disclosed herein to the eye of the subject to an ocular secretory gland of the subject. In one aspect, the disclosure relates to a method of treating an ocular condition in a subject in need thereof, the method comprising administering an effective amount of the pharmaceutical composition as disclosed herein to the eye of the subject to an ocular secretory gland of the subject.

In some embodiments, the rAAV virion is administered to at least one eye and/or lacrimal gland of the subject. In some embodiments, cells within the at least one eye and/or lacrimal gland are transduced by the rAAV virion. In some embodiments, the transduced cells within the at least one eye and/or lacrimal gland express a therapeutically effective amount of the neurotrophic factor into the tear film and optionally onto the ocular surface of the subject. In some embodiments, the transduced cells within the at least one eye and/or lacrimal gland express an effective amount of the neurotrophic factor into the tear film and optionally onto the ocular surface of the subject.

In some embodiments, the rAAV virion may be administered repeatedly to at least one eye and/or lacrimal gland of the subject to ensure that the gland is secreting an appropriate amount of target (protein, peptide, enzyme, etc) into the tear film. In some embodiments, cells within the at least one eye and/or lacrimal gland are transduced by the rAAV virion. In some embodiments, the transduced cells within the at least one eye and/or lacrimal gland express a therapeutically effective amount of the neurotrophic factor into the tear film and optionally onto the ocular surface of the subject. In some embodiments, the transduced cells within the at least one eye and/or lacrimal gland express an effective amount of the neurotrophic factor into the tear film and optionally onto the ocular surface of the subject.

In some embodiments, the ocular condition is neurotrophic keratitis. In some embodiments, one or more symptoms of the ocular condition are reduced compared to the symptoms of the ocular condition before administration of the rAAV virion. In some embodiments, one or more symptoms of the ocular condition are reduced compared to the symptoms of the ocular condition in an untreated control subject. In some embodiments, one or more symptoms of the ocular condition are reduced compared to the symptoms of the ocular condition in a contralateral eye.

Also provided are rAAV virions and pharmaceutical compositions described herein for use in the preparation of a medicament for the treatment of an ocular condition.

In some aspects, the disclosure provides a method of treating an ocular condition in a subject in need thereof, the method comprising administering a recombinant adeno-associated virus (rAAV) virion, the rAAV virion comprising an AAV capsid and an expression cassette comprising a polynucleotide encoding a neurotrophic factor operatively linked to a promoter, to at least one eye of the subject or to at least one lacrimal gland of the eye of the subject.

In the embodiments described herein, the rAAV virion is administered to a lacrimal gland of the subject. In some embodiments, the lacrimal gland is the main lacrimal gland or any one of the Wolfring's glands or the Krause's glands of the subject. In some embodiments, the lacrimal gland is the main lacrimal gland.

In the embodiments described herein, cells within the lacrimal gland are transduced by the rAAV virion. In some embodiments, the transduced cells within the lacrimal gland express an effective amount of the neurotrophic factor into the tear film and optionally onto the ocular surface of the subject. In some embodiments, the neurotrophic factor is a Nerve Growth Factor (NGF) protein. In some embodiments, the polynucleotide encoding the NGF protein comprises a sequence that shares at least 95% identity to SEQ ID NO: 2. In some embodiments, the NGF protein comprises a sequence that shares at least 95% identity to SEQ ID NO: 1. In some embodiments, the NGF protein comprises SEQ ID NO: 1. In some embodiments, the polynucleotide encoding the NGF protein comprises SEQ ID NO: 2.

In the embodiments described herein, the neurotrophic factor is a Glial Derived Neurotrophic Factor (GDNF) protein. In some embodiments, the polynucleotide encoding the GDNF protein comprises a sequence that shares at least 95% identity to SEQ ID NO: 4. In some embodiments, the GDNF protein comprises a sequence that shares at least 95% identity to SEQ ID NO: 3. In some embodiments, the GDNF protein comprises SEQ ID NO: 3. In some embodiments, the polynucleotide encoding the GDNF protein comprises SEQ ID NO: 4.

In the embodiments described herein, the AAV capsid shares at least 95%, 98%, or 100% identity to AAV2 VP1 (SEQ ID NO: 6), AAV2 VP3 (SEQ ID NO: 8), AAV5 (SEQ ID NO: 10), AAV8 (SEQ ID NO: 12), or AAV9 (SEQ ID NO: 14).

In the embodiments described herein, the promoter is a CAG promoter (SEQ ID NO: 5).

In the embodiments described herein, the ocular condition is a chemical burn of the ocular surface, corneal wound, corneal ulcer, persistent epithelial defect, dry eye disease, neurotrophic keratitis, herpes simplex viral infection of the trigeminal nerve and/or the eye, varicella zoster virus infection of the trigeminal nerve and/or the eye, or diabetic complications of the corneal nerves.

In the embodiments described herein, one or more symptoms of the ocular condition are reduced compared to the symptoms of the ocular condition before administration of the rAAV virion. In some embodiments, one or more symptoms of the ocular condition are reduced compared to the symptoms of the ocular condition in an untreated control subject. In some embodiments, one or more symptoms of the ocular condition are reduced compared to the symptoms of the ocular condition in a contralateral eye.

In some embodiments, the disclosure provides a recombinant adeno-associated virus (rAAV) virion, comprising an AAV capsid and an expression cassette, wherein the expression cassette comprises a polynucleotide encoding a neurotrophic factor operatively linked to a promoter.

In the embodiments described herein, the neurotrophic factor is a Nerve Growth Factor (NGF) protein. In some embodiments, the polynucleotide encoding the NGF protein comprises a sequence that shares at least 95% identity to SEQ ID NO: 2 or SEQ ID NO: 17. In some embodiments, the NGF protein comprises a sequence that shares at least 95% identity to SEQ ID NO: 1. In some embodiments, the NGF protein comprises SEQ ID NO: 1. In some embodiments, the polynucleotide encoding the NGF protein comprises SEQ ID NO: 2 or SEQ ID NO: 17.

In the embodiments described herein, the neurotrophic factor is a Glial Derived Neurotrophic Factor (GDNF) protein. In some embodiments, the polynucleotide encoding the GDNF protein comprises a sequence that shares at least 95% identity to SEQ ID NO: 4 or SEQ ID NO: 18.

In some embodiments, the GDNF protein comprises a sequence that shares at least 95% identity to SEQ ID NO: 3. In some embodiments, the GDNF protein comprises SEQ ID NO: 3. In some embodiments, the polynucleotide encoding the GDNF protein comprises SEQ ID NO: 4 or SEQ ID NO: 18.

In the embodiments described herein, the AAV capsid comprises a VP3 that shares at least 95%, 98%, or 100% identity to AAV2 VP1 (SEQ ID NO: 6), AAV2 VP3 (SEQ ID NO: 8), AAV5 (SEQ ID NO: 10), AAV8 (SEQ ID NO: 12), or AAV9 (SEQ ID NO: 14).

In the embodiments described herein, the promoter is a CAG promoter (SEQ ID NO: 5 or a CMV promoter (SEQ ID NO: 16).

In the embodiments described herein, the expression cassette comprises a sequence that shares at least 90%, 95%, 96%, 97%, 98%, 99%, or 100% identity to SEQ ID NO: 23.

In the embodiments described herein, the expression cassette comprises a sequence that shares at least 90%, 95%, 96%, 97%, 98%, 99%, or 100% identity to SEQ ID NO: 24.

In the embodiments described herein, the expression cassette comprises a sequence that shares at least 90%, 95%, 96%, 97%, 98%, 99%, or 100% identity to SEQ ID NO: 25.

In some embodiments, the disclosure provides a recombinant adeno-associated virus (rAAV) virion comprising an expression cassette comprising a polynucleotide of SEQ ID NO: 25.

In the embodiments described herein, the rAAV comprises an AAV capsid.

In the embodiments described herein, the AAV capsid shares at least 95%, 98%, or 100% identity to AAV2 VP1 (SEQ ID NO: 6), AAV2 VP3 (SEQ ID NO: 8), AAV5 (SEQ ID NO: 10), AAV8 (SEQ ID NO: 12), or AAV9 (SEQ ID NO: 14).

In some embodiments, the disclosure provides a composition comprising an rAAV virion, wherein the rAAV virion comprises:
(a) an AAV capsid, and
(b) an expression cassette, wherein the expression cassette comprises a polynucleotide sharing at least 95% identity to SEQ ID NO: 2 or SEQ ID NO: 17, and wherein the polynucleotide is linked to a promoter.

In some embodiments, the disclosure provides a composition comprising an rAAV virion, wherein the rAAV virion comprises:
(a) an AAV capsid, and
(b) an expression cassette, wherein the expression cassette comprises a polynucleotide sharing at least 95% identity to SEQ ID NO: 4 or SEQ ID NO: 18, and wherein the polynucleotide is linked to a promoter.

In some embodiments, the disclosure provides a composition comprising an rAAV virion, wherein the rAAV virion comprises:
(a) an AAV2, AAV5, AAV8, or AAV9 capsid, and
(b) an expression cassette, wherein the expression cassette comprises a polynucleotide sharing at least 95% identity to SEQ ID NO: 2 or SEQ ID NO: 17, and wherein the polynucleotide is linked to a promoter.

In some embodiments, the disclosure provides a composition comprising an rAAV virion, wherein the rAAV virion comprises:
(a) an AAV2, AAV5, AAV8, or AAV9 capsid, and
(b) an expression cassette, wherein the expression cassette comprises a polynucleotide sharing at least 95% identity to SEQ ID NO: 4 or SEQ ID NO: 18, and wherein the polynucleotide is linked to a promoter.

In the embodiments described herein, the AAV capsid shares at least 95%, 98%, or 100% identity to AAV2 VP1 (SEQ ID NO: 6), AAV2 VP3 (SEQ ID NO: 8), AAV5 (SEQ ID NO: 10), AAV8 (SEQ ID NO: 12), or AAV9 (SEQ ID NO: 14). In some embodiments, the AAV capsid is AAV2. In some embodiments, the AAV capsid is AAV5. In some embodiments, the AAV capsid is AAV9.

In some embodiments, the disclosure provides a composition comprising an rAAV virion, wherein the rAAV virion comprises:
(a) an AAV2 capsid, and
(b) an expression cassette, wherein the expression cassette comprises a polynucleotide sharing at least 95% identity to SEQ ID NO: 25.

In some embodiments, the disclosure provides a composition comprising an rAAV virion, wherein the rAAV virion comprises:
(a) an AAV5 capsid, and
(b) an expression cassette, wherein the expression cassette comprises a polynucleotide sharing at least 95% identity to SEQ ID NO: 25.

In some embodiments, the disclosure provides a composition comprising an rAAV virion, wherein the rAAV virion comprises:
(a) an AAV9 capsid, and
(b) an expression cassette, wherein the expression cassette comprises a polynucleotide sharing at least 95% identity to SEQ ID NO: 25.

In some embodiments, the disclosure provides a pharmaceutical composition comprising an rAAV virion or composition described herein, and a pharmaceutically acceptable carrier.

In the embodiments described herein, the composition comprises about 1×10⁷ to about 1×10¹⁴ genome copies per milliliter of the rAAV virion.

In the embodiments described herein, the composition comprises about 1×10¹² to about 6.2×10¹² genome copies per milliliter of the rAAV virion.

In the embodiments described herein, the composition is formulated for administration into the lacrimal gland. In some embodiments, the composition is formulated for administration onto the ocular surface.

In the embodiments described herein, the composition is formulated for use, or adaptable for use, in the treatment of an ocular disease, disorder, or condition.

In some embodiments, the disclosure provides a method of treating an ocular condition in a subject in need thereof, the method comprising administering an effective amount of a pharmaceutical composition described herein to the eye of the subject to an ocular secretory gland of the subject.

In the embodiments described herein, the rAAV virion is administered to the lacrimal gland of the subject. In some embodiments, cells within the lacrimal gland are transduced by the rAAV virion. In some embodiments, the transduced cells within the lacrimal gland express an effective amount of the neurotrophic factor into the tear film and optionally onto the ocular surface of the subject.

In the embodiments described herein, the ocular condition is neurotrophic keratitis.

In the embodiments described herein, one or more symptoms of the ocular condition are reduced compared to the symptoms of the ocular condition before administration of the rAAV virion.

In some embodiments, one or more symptoms of the ocular condition are reduced compared to the symptoms of the ocular condition in an untreated control subject.

In the embodiments described herein, one or more symptoms of the ocular condition are reduced compared to the symptoms of the ocular condition in a contralateral eye.

In some embodiments, the disclosure provides for use of an rAAV virion, composition, or pharmaceutical composition of any one of foregoing or related aspects for use in a method of any one of the foregoing or related aspects.

In some embodiments, the disclosure provides for use of an rAAV or composition of any of the foregoing or related aspects, in the manufacture of a medicament for use in the method of any one of the foregoing or related aspects.

In some embodiments, the disclosure provides a kit comprising an rAAV virion, composition, or pharmaceutical composition of any of the foregoing or related aspects, and instructions for use in the method of any one of the foregoing or related aspects.

In some embodiments, the disclosure provides a recombinant adeno-associated virus (rAAV) virion for use in a method of treating an ocular condition in a subject in need thereof, the method comprising administering a recombinant adeno-associated virus (rAAV) virion, the rAAV virion comprising an AAV capsid and an expression cassette comprising a polynucleotide encoding a neurotrophic factor operatively linked to a promoter, to at least one eye of the subject or to at least one lacrimal gland of the eye of the subject.

In the embodiments described herein, the rAAV virion is administered to a lacrimal gland of the subject.

In the embodiments described herein, cells within the lacrimal gland are transduced by the rAAV virion.

In the embodiments described herein, the neurotrophic factor is a Nerve Growth Factor (NGF) protein. In some embodiments, the neurotrophic factor is a Glial Derived Neurotrophic Factor (GDNF) protein.

In the embodiments described herein, the ocular condition is a chemical burn of the ocular surface, corneal wound, persistent epithelial defect, dry eye disease, neurotrophic keratitis, herpes simplex viral infection of the trigeminal nerve and/or the eye, varicella zoster virus infection of the trigeminal nerve and/or the eye, or diabetic complications of the corneal nerves.

### BRIEF DESCRIPTION OF FIGURES

FIG. 1A-FIG. 1C show illustrative plasmids containing rAAV expression cassettes of the disclosure with inverted terminal repeats (ITRs), a CAG promoter, and glial-derived neurotrophic factor (GDNF)(FIG. 1A), nerve growth factor (NGF)(FIG. 1B), or enhanced green fluorescent protein (eGFP)(FIG. 1C) transgene polynucleotide elements.

FIGS. 2A-2B each show an example of delivery of a viral vector to the lacrimal gland of a human subject.

FIG. 3 shows an example of the visual analog scale used in the Eye Dryness test for subjects to record the severity of their symptoms.

FIG. 4 shows a grading diagram of the division of the corneal surface based on the NEI/Industry Workshop scale.

FIG. 5 shows the Mackie Classification of Neurotrophic Keratitis

FIGS. 6A-6K are images of lacrimal tissue stained with anti-eGFP antibody. Lacrimal glands were dosed with rAAV virions containing an expression cassettes with eGFP transgene by intralacrimal injection. Lacrimal tissue was stained with anti-eGFP antibody to assess eGFP expression. Black arrows indicate staining showing eGFP expression.

FIGS. 7A-7E are images of lacrimal tissue stained with anti-hNGF antibody. Lacrimal glands were dosed with rAAV virions (AAV9 serotype) engineered to deliver an expression cassettes with hNGF transgene by intralacrimal injection. Lacrimal tissue was stained with anti-hNGF antibody to assess hNGF expression. Black arrows indicate staining showing hNGF expression.

FIG. 8 is a plot showing the concentration of hNGF in tear film in animals treated with AAV.hNGF. Protein concentration was measured using Schirmer strips to collect tear film from Dutch-belted rabbits at the indicated number of days following intralacrimal administration of AAV2.hNGF, AAV5.hNGF, AAV9.hNGF, or control (no injection of AAV).

FIG. 9 shows a schematic diagram depicting the elements between the ITRs of an AAV plasmid. The plasmid encodes EGFP linked to a secretion signal at its N-terminus (“secEGFP”) under control of a CMV promoter. The woodchuck hepatitis virus post-translational regulatory element (WPRE) serves to increase transgene expression and is proximal to the bovine growth hormone polyadenylation (pA) signal.

FIG. 10 provides images of porcine lacrimal gland that received an injection of AAV-secEGFP (AAV2 or AAV9 serotype) and was harvested on Day 103 and fixed in paraffin. IHC of 5 μM sections was performed using anti-GFP antibody and DAPI (nuclear) counterstain. Images were captured using a confocal microscope at 100× magnification. Negative control animals received no injection.

FIG. 11 provides an image of porcine lacrimal gland that received an injection of AAV9-secEGFP and was harvested on Day 103 and fixed in paraffin. IHC of 5 μM sections was performed using anti-GFP antibody and DAPI (nuclear) counterstain. In addition to lacrimal gland acinar cells, ductile epithelial cells appear to be transduced by AAV9 (white arrows).

FIG. 12 provides an image of porcine lacrimal gland that received an injection of AAV was harvested at Day 103 and fixed in paraffin. H&E staining of 5 μM paraffin sections at 100× reveal no inflammatory infiltrate, macro, or micro abnormalities.

FIGS. 13A-13C are plots showing concentration (pg/mL) of hNFGB in tear film of pigs. Tears were collected from the left eye of pigs (n=4) on Day 7, 14, 21, 28, and 35 after transduction with an adeno-associated viral vector (AAV) encoding human nerve growth factor (AAV-hNGFβ). AAV2-hNGFβ (FIG. 13A) and AAV5-hNGFβ (FIG. 13B), had a detectable levels of hNGFβ within the standard curve range of the MSD assay 7 days after transduction. AAV9-hNGFβ (FIG. 13C) had tear levels of hNGFβ above the level of quantitation for the upper limit of the MSD assay 7 days after transduction.

FIG. 14 shows an illustrative plasmid containing an rAAV expression cassette of SEQ ID NO: 25 with inverted terminal repeats (ITRs), a CAG promoter, and nerve growth factor (NGF) transgene polynucleotide elements.

### DETAILED DESCRIPTION

### Overview

The present disclosure provides methods of treating an ocular condition in a subject in need thereof. In healthy subjects, neurotrophic factors provide maintenance and repair to the cornea. Corneal epithelial cells release the soluble neurotrophic factors NGF and GDNF on the ocular surface, promoting survival and healing of corneal cells through activation of the TrkA and GFRa-1 receptors, respectively. NGF and GDNF are constitutively present in tear film, and increase in abundance during the response to irritation or damage to the cornea.

As disclosed herein, AAV-based delivery of transgenes to the eye provides a method to treat ocular disorders. rAAV virions have distinct advantages for transgene delivery. A transgene delivered by an rAAV virion is likely to be incorporated into the genome of the transduced cell, allowing potential long-term expression of the transgene product. In addition, rAAV virions are not as immunogenic as other viral delivery vectors, such as adenovirus. AAV-based delivery vectors using subretinal and intravitreal injection to transduce cells in the posterior segment of the eye are described and show efficacy in vivo (U.S. Pat. No. 10,308,957; Petrs-Silva et al., Mol Ther. 19:293-301 (2011); Rodriques et al., Pharm Res. 36:29 (2019)).

AAV serotypes used in AAV-based delivery of transgene to the eye include AAVI, AAV2, AAV4, AAV5, AAV6, AAV7, AAV8, and AAV9 used to deliver transgene (Lebherz et al. J Gene Med.; 10 (4): 375-382 (2008)). AAV serotypes used in AAV-based delivery of a transgene to the lacrimal gland include AAV2, AAV4, AAV5, AAV5w8, AAV x5, AAV 9, AAV 12, and BAAV (Rocha et al., Invest Ophthalmol Vis Sci. 52:9567-9572 (2011)).

Viral vector-based delivery of transgenes encoding a protein product by injection into the lacrimal gland (i.e. intralacrimal delivery, intralacrimal injection, or intralacrimal administration) can be advantageous for treating ocular diseases and symptoms. Proteins produced in the lacrimal gland and secreted in the lacrimal fluid of a subject may be improved due to post-translational modification (PTM) (covalent and generally enzymatic modification of proteins following protein biosynthesis) whereby the proteins are synthesized by ribosomes translating mRNA into polypeptide chains, which may then undergo PTM to form the mature protein product. PTMs may confer important properties in cell signaling that would otherwise be absent with topical delivery of recombinant human proteins that are often made in bacteria such as escherichia coli. In addition, when secreted with natural tear film components that may contain co-factors, chaperones, enzymes or other proteins, administration via the lacrimal fluid may confer biologic activity that could not be achieved with only administration of topically administered protein alone.

In one aspect, an rAAV virion is provided for the expression of a neurotrophic factor. In some embodiments, cells of the at least one eye and/or lacrimal gland are transduced with the rAAV virion of the disclosure. The lacrimal glands are the major source of tear fluid responsible for promoting a healthy ocular surface and maintaining normal visual function. The main lacrimal gland comprises palpebral and orbital lobes, which are continuous with each other at the lateral edge of the aponeurosis of levator palpebrae superioris. The lobules have many acini and intralobular ducts that form excretory ducts that open into the fornix of the conjunctiva. The main lacrimal gland is comprised of acinar cells, ductal cells, and/or myoepithelial cells (Obata Cornea.; 25 (10 Suppl 1):S82-9 (2006)). The main lacrimal gland secretes an aqueous layer of tear film onto the ocular surface of the eye of a subject. As used herein, the term “lacrimal gland” refers to the main lacrimal gland, as well as the Wolfring's glands and the Krause's glands of a subject. The accessory glands, known as the Wolfring's glands and Krause's glands are located in the eyelid. In the upper eyelid there are about 2 to 5 Wolfring's glands and about forty Krause's glands. In the lower eyelid there are about 6 to 8 Krause's glands. Specific location and anatomy of the lacrimal functional unit is well-known (Conrady et al., J Ophthalmol. Article ID 7542929 (2016)).

### Expression Cassette

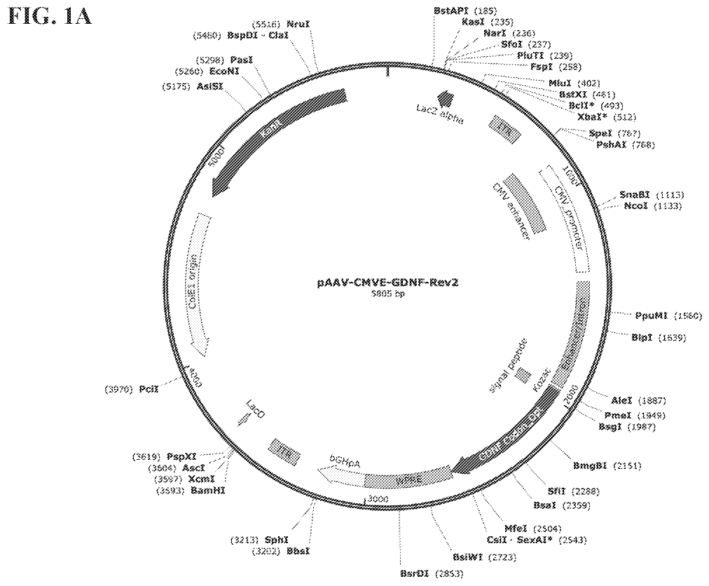

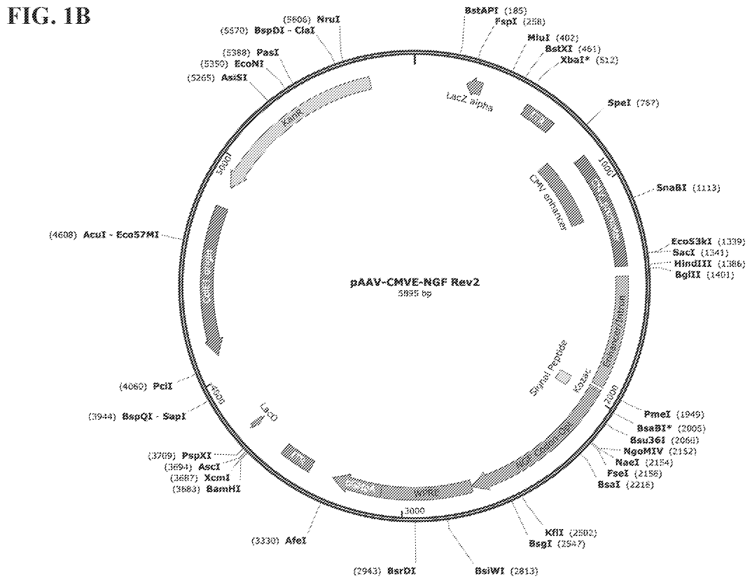

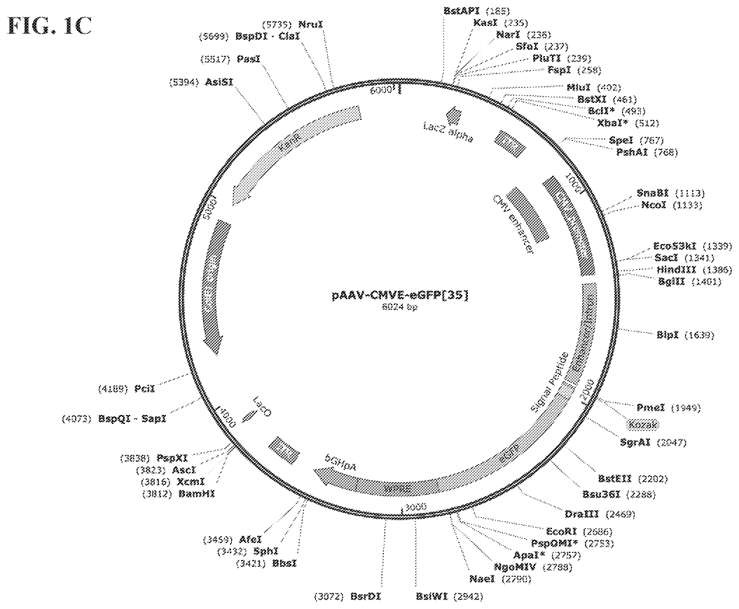

The rAAV virions of the disclosure may comprise an expression cassette. As used herein, the term “expression cassette” refers to a polynucleotide comprising at least one polynucleotide sequence encoding a protein of interest or transgene, e.g., a neurotrophic factor, flanked by inverted terminal repeats. The expression cassette may further comprise other polynucleotide sequences, e.g., promoters, regulatory elements (e.g., one or more promoters or enhancers), translation initiation sequences, coding sequences, and termination sequences (FIGS. 1A-1C and FIG. 14). Regulatory elements may be operably linked to the transgene and promote expression.

In some embodiments, the expression cassette of the present disclosure comprises a polynucleotide sequence encoding a neurotrophic factor. In some embodiments, the expression cassette provides increased expression of a neurotrophic factor in the at least one eye and/or lacrimal glands. In some embodiments, expression of the neurotrophic factor may be increased 5%, 10%, 15%, 20%, or 25% compared to expression of the neurotrophic factor in an untreated subject, or in the contralateral eye of a treated subject. As used herein, “subject” means any mammal, including mice, rabbits, pigs, canines, non-human primates (NHP) and humans. In some embodiment, the subject is a human or NHP. Moreover, “individual” or “patient” may be used interchangeably with “subject.” In some embodiments, expression of the neurotrophic factor may be increased 1-fold, 2-fold, 3-fold, 4-fold, or 5-fold compared to expression of the neurotrophic factor in an untreated subject, or in the contralateral eye of a treated subject. In some embodiments, expression of the neurotrophic factor is increased 5%, 10%, 15%, 20%, 25%, 30%, 35%, 40%, 45%, 50%, 55%, 60%, 65%, 70%, 75%, 80%, 85%, 90%, 95%, or 99% compared to expression of the neurotrophic factor in an untreated subject, or in the contralateral eye of a treated subject. In some embodiments, expression of the neurotrophic factor may be increased 1-fold, 2-fold, 3-fold, 4-fold, or 5-fold, 6-fold, 7-fold, 8-fold, or 9-fold compared to expression of the neurotrophic factor in an untreated subject, or in the contralateral eye of a treated subject. In some embodiments, the neurotrophic factor may be expression at any detectable level in the treated eye, whereas the neurotrophic factor may be not be expressed, or expressed at undetectable levels, in an untreated subject, or in the contralateral eye of a treated subject. Put another way, the eye or lacrimal gland to which the rAAV virion is administered may express a neurotrophic factor in higher abundance than in an eye or lacrimal gland that has only endogenous (i.e., native) expression of the neurotrophic factor or in eyes that have a lower or impaired secretion of endogenous (i.e., native) expression of the neurotrophic factor.

In some embodiments, the neurotrophic factor is nerve growth factor (NGF) protein or a functional variant thereof. As used herein, “NGF”, “NGFβ”, or “Nerve Growth Factor Beta” refer to Nerve Growth Factor which is a gene that encodes a protein which homodimerizes and performs nerve growth stimulating activity. NGF is also involved in the regulation and differentiation of neurons. As used herein, the term “NGF protein” refers to an NGF protein from any species. The term “functional variant” refers to variants having sequence substitutions, insertions, deletions, and/or N- or C-terminal truncations, where the functional variant retains one or more functions of the reference protein, e.g., a native NGF protein. NGF is a 26-kDa polypeptide involved primarily in the growth, maintenance, proliferation, and survival of nerve cells. In the cornea, NGF binds its receptor trkA^{NGFR} and p75^{NTR}, eliciting signal transduction pathways associated with healing of both cornea and conjunctiva (Micera et al., Exp Eye Res. 83:747-57 (2006)). In some embodiments, the NGF protein comprises SEQ ID NO. 1. In some embodiments, the NGF protein comprises a sequence at least 80%, 85%, 90%, 95%, 96%, 97%, 98%, 99%, or 100% identical to SEQ ID NO: 1. In some embodiments, the NGF protein shares a sequence with at least 80%, at least 85%, at least 90%, at least 95%, at least 96%, at least 97%, at least 98%, at least 99%, or 100% identity to SEQ ID NO: 1. In some embodiments, the polynucleotide encoding the NGF protein comprises SEQ ID NO: 2. In some embodiments, the polynucleotide encoding the NGF protein comprises a sequence at least 80%, 85%, 90%, 95%, 96%, 97%, 98%, 99%, or 100% identical to SEQ ID NO: 2. In some embodiments, the polynucleotide encoding the NGF protein is encoded by a sequence that shares at least 80%, at least 85%, at least 90%, at least 95%, at least 96%, at least 97%, at least 98%, at least 99%, or 100% identity to SEQ ID NO: 2. In some embodiments, the polynucleotide encoding the NGF protein is codon optimized. In some embodiments, the polynucleotide encoding the NGF protein comprises SEQ ID NO: 17. In some embodiments, the polynucleotide encoding the NGF protein comprises a sequence at least 80%, 85%, 90%, 95%, 96%, 97%, 98%, 99%, or 100% identical to SEQ ID NO: 17. In some embodiments, the polynucleotide encoding the NGF protein is encoded by a sequence that shares at least 80%, at least 85%, at least 90%, at least 95%, at least 96%, at least 97%, at least 98%, at least 99%, or 100% identity to SEQ ID NO: 17.

>Codon optimized NGF
(SEQ ID NO: 17)
atgagcatgctgttttacaccctgatcaccgccttcctgatcggca
tccaggccgagccccacagcgagagcaacgtgcccgctggacaca
caatccctcaggcccactggacaaaactgcagcatagcctggata
cagccctgagaagggccagaagcgcccctgccgccgccatcgccg
ctagagtggccggccagacaagaaatatcaccgtggacccccggc
tgttcaagaaaagaagactgcggtctcctagagtgctcttttcaa
cacagcctcctcgggaagccgctgatacccaggacctggacttcg
aggtgggaggcgccgctcctttcaatagaacccacagatccaaga
gaagctctagccaccctatcttccaccggggcgagttcagcgtgt
gcgacagcgtttctgtgtgggtgggagataagaccaccgcaaccg
atatcaagggcaaggaagtgatggtgctgggcgaagtgaacatca
acaacagcgtctttaagcagtacttcttcgagacaaagtgccggg
acccaaaccccgtggacagcggctgcagaggcattgactccaagc
actggaactcctactgcaccacaacacacaccttcgtgaaggccc
tgaccatggacggcaaacaagctgcctggcggttcatcagaatcg
acaccgcctgtgtctgtgtgctgagcagaaaggccgtgcgccggg
cctag

In some embodiments, the NGF protein is a human NGF protein.

In some embodiments, the neurotrophic factor is glial derived neurotrophic factor (GDNF) protein. As used herein, the term “GDNF protein” refers to a GDNF protein from any species. The term, “functional variant” refers to variants having sequence substitutions, insertions, deletions, and/or N- or C-terminal truncations, where the functional variant retains one or more functions of the reference protein, e.g., a native GDNF protein. GDNF is a 21 kDa protein that supports the growth, maintenance, and differentiation of a wide variety of neuronal systems by GDNF acting on its receptor GFRα-1 (Qi et al., Br J Ophthalmol. 92:1269-74 (2008)). In some embodiments, the GDNF protein comprises SEQ ID NO: 3. In some embodiments, the GDNF protein comprises a sequence at least 80%, 85%, 90%, 95%, 96%, 97%, 98%, 99%, or 100% identical to SEQ ID NO: 3. In some embodiments, the GDNF protein shares a sequence with at least 80%, at least 85%, at least 90%, at least 95%, at least 96%, at least 97%, at least 98%, at least 99%, or 100% identity to SEQ ID NO: 3. In some embodiments, the polynucleotide encoding the GDNF protein comprises SEQ ID NO: 4. In some embodiments, the polynucleotide encoding the GDNF protein comprises a sequence at least 80%, 85%, 90%, 95%, 96%, 97%, 98%, 99%, or 100% identical to SEQ ID NO: 4. In some embodiments, the GDNF protein is encoded by a sequence that shares a sequence at least 80%, at least 85%, at least 90%, at least 95%, at least 96%, at least 97%, at least 98%, at least 99%, or 100% identity to SEQ ID NO: 4. In some embodiments, the polynucleotide encoding the GDNF protein comprises SEQ ID NO: 18. In some embodiments, the polynucleotide encoding the GDNF protein comprises a sequence at least 80%, 85%, 90%, 95%, 96%, 97%, 98%, 99%, or 100% identical to SEQ ID NO: 18. In some embodiments, the polynucleotide encoding the GDNF protein is codon optimized. In some embodiments, the polynucleotide encoding the GDNF protein is encoded by a sequence that shares at least 80%, at least 85%, at least 90%, at least 95%, at least 96%, at least 97%, at least 98%, at least 99%, or 100% identity to SEQ ID NO: 18.

>Codon Optimized GDNF
(SEQ ID NO: 18)
atgaactgtgggacgttgtggccgtgtgcctggtcctcctgcaca
ccgcctccgcctttccactgcctgctggaaagcggcctcctgagg
cccctgctgaagatagaagcctgggcagaagaagagcccccttcg
ccctgagctctgatagcaacatgcctgaggactaccccgaccagt
tcgacgacgtgatggactttatccaggccaccatcaagagactga
aacggagccctgacaagcagatggccgtgctgcctagacgggaaa
gaaatagacaggccgcagctgccaaccccgagaacagcagaggca
aaggccggagaggccagaggggaaaaaacagaggatgtgtgctga
ccgccatccatctgaacgtgacagatctgggcctgggctatgaga
ccaaggaagagctgatcttcagatactgcagcggcagctgcgacg
ccgccgagacaacctacgacaagatcctgaagaacctgtcccgga
atcggcggctggtgtctgacaaggtgggccaagcttgctgcagac
caattgccttcgacgatgatctgtctttcctggatgacaacctgg
tgtaccacatcctgagaaagcacagcgccaagcgctgcggctgta
tctag

In some embodiments, the GDNF is human GDNF.

TABLE 1
Sequences of Neurotrophic Factors

| | | Nucleotide |
|---|---|---|
| | Protein | (Open Reading Frame) |
| NGF | MSMLFYTLITAFLIGIQAEP | ATGTCCATGTTGTTCTACAC |
| | HSESNVPAGHTIPQVHWTKL | TCTGATCACAGCTTTTCTGA |
| | QHSLDTALRRARSAPAAAIA | TCGGCATACAGGCGGAACCA |
| | ARVAGQTRNITVDPRLFKKR | CACTCAGAGAGCAATGTCCC |
| | RLRSPRVLFSTQPPREAADT | TGCAGGACACACCATCCCCC |
| | QDLDFEVGGAAPFNRTHRSK | AAGTCCACTGGACTAAACTT |
| | RSSSHPIFHRGEFSVCDSVS | CAGCATTCCCTTGACACTGC |
| | VWVGDKTTATDIKGKEVMVL | CCTTCGCAGAGCCCGCAGCG |
| | GEVNINNSVFKQYFFETKCR | CCCCGGCAGCGGCGATAGCT |
| | DPNPVDSGCRGIDSKHWNSY | GCACGCGTGGCGGGGCAGAC |
| | (SEQ ID NO: 1) | CCGCAACATTACTGTGGACC |
| | | CCAGGCTGTTTAAAAAGCGG |
| | | CGACTCCGTTCACCCCGTGT |
| | | GCTGTTTAGCACCCAGCCTC |
| | | CCCGTGAAGCTGCAGACACT |
| | | CAGGATCTGGACTTCGAGGT |
| | | CGGTGGTGCTGCCCCCTTCA |
| | | ACAGGACTCACAGGAGCAAG |
| | | CGGTCATCATCCCATCCCAT |
| | | CTTCCACAGGGGCGAATTCT |
| | | CGGTGTGTGACAGTGTCAGC |
| | | GTGTGGGTTGGGGATAAGAC |
| | | CACCGCCACAGACATCAAGG |
| | | GCAAGGAGGTGATGGTGTTG |
| | | GGAGAGGTGAACATTAACAA |
| | | CAGTGTATTCAAACAGTACT |
| | | TTTTTGAGACCAAGTGCCGG |
| | | GACCCAAATCCCGTTGACAG |
| | | CGGGTGCCGGGGCATTGACT |
| | | CAAAGCACTGGAACTCATAT |
| | | TGTACCACGACTCACACCTT |
| | | TGTCAAGGCGCTGACCATGG |
| | | ATGGCAAGCAGGCTGCCTGG |
| | | CGGTTTATCCGGATAGATAC |
| | | GGCCTGTGTGTGTGTGCTCA |
| | | GCAGGAAGGCTGTGAGAAGA |
| | | GCCTGA |
| | | (SEQID NO: 2) |
| GDNF | MKLWDVVAVCLVLLHTASAF | ATGAAGTTATGGGATGTCGT |
| | PLPAGKRPPEAPAEDRSLGR | GGCTGTCTGCCTGGTGCTGC |
| | RRAPFALSSDSNMPEDYPDQ | TCCACACCGCGTCCGCCTTC |
| | FDDVMDFIQATIKRLKRSPD | CCGCTGCCCGCCGGTAAGAG |
| | KQMAVLPRRERNRQAAAANP | GCCTCCCGAGGCGCCCGCCG |
| | ENSRGKGRRGQRGKNRGCVL | AAGACCGCTCCCTCGGCCGC |
| | TAIHLNVTDLGLGYETKEEL | CGCCGCGCGCCCTTCGCGCT |
| | IFRYCSGSCDAAETTYDKIL | GAGCAGTGACTCAAATATGC |
| | KNLSRNRRLVSDKVGQACCR | CAGAGGATTATCCTGATCAG |
| | PIAFDDDLSFLDDNLVYHIL | TTCGATGATGTCATGGATTT |
| | RKHSAKRCGCI | TATTCAAGCCACCATTAAAA |
| | (SEQ ID NO: 3) | GACTGAAAAGGTCACCAGAT |
| | | AAACAAATGGCAGTGCTTCC |
| | | TAGAAGAGAGCGGAATCGGC |
| | | AGGCTGCAGCTGCCAACCCA |
| | | GAGAATTCCAGAGGAAAAGG |
| | | TCGGAGAGGCCAGAGGGGCA |
| | | AAAACCGGGGTTGTGTCTTA |
| | | ACTGCAATACATTTAAATGT |
| | | CACTGACTTGGGTCTGGGCT |
| | | ATGAAACCAAGGAGGAACTG |
| | | ATTTTTAGGTACTGCAGCGG |
| | | CTCTTGCGATGCAGCTGAGA |
| | | CAACGTACGACAAAATATTG |
| | | AAAAACTTATCCAGAAATAG |
| | | AAGGCTGGTGAGTGACAAAG |
| | | TAGGGCAGGCATGTTGCAGA |
| | | CCCATCGCCTTTGATGATGA |
| | | CCTGTCGTTTTTAGATGATA |
| | | ACCTGGTTTACCATATTCTA |
| | | AGAAAGCATTCCGCTAAAAG |
| | | GTGTGGATGTATCTGA |
| | | (SEQ ID NO: 4) |

In some embodiments, the expression cassette of the present disclosure comprises a promoter. The term “promoter” as used herein refers to a DNA sequence that directs the binding of RNA polymerase and thereby promotes RNA synthesis, i.e., a minimal sequence sufficient to direct transcription. A promoter can be operably linked to a transgene, e.g., a neurotrophic factor. Transcription of a transgene can be initiated and regulated by the promoter to which it is operably linked. For example, an expression cassette comprising a promoter operably linked to a transgene will express the transgene if the RNA synthesis initiated at the promoter. Promoters and corresponding protein or polypeptide expression may be ubiquitous, meaning strongly active in a wide range of cells, tissues and species or cell-type specific, tissue-specific, or species specific. Promoters may be “constitutive,” meaning continually active, or “inducible,” meaning the promoter can be activated or deactivated by the presence or absence of biotic or abiotic factors. Also included in the nucleic acid constructs or vectors of the invention are enhancer sequences that may or may not be contiguous with the promoter sequence. Enhancer sequences influence promoter-dependent gene expression and may be located in the 5′ or 3′ regions of the native gene.

Any suitable promoter region or promoter sequence therein can be used in the subject polynucleotide cassettes, so long as the promoter region promotes expression of a polynucleotide sequence encoding an NGF or a GNDF protein in the at least one eye and/or lacrimal glands. In some embodiments, the promoter promotes expression of the gene in mammalian eye and/or lacrimal glands. In some embodiments, the expression cassette comprises a cell-type specific promoter. The promoter may specifically promote transcription in the cells of the eye and/or the cells of the lacrimal gland.

In some embodiments, the promoter is a CAG promoter. In some embodiments, the promoter comprises SEQ ID NO: 5. In some embodiments, the promoter comprises a sequence at least 95%, 96%, 97%, 98%, 99%, or 100% identical to SEQ ID NO: 5. In some embodiments, the promoter shares at least 90%, 95%, 96%, 97%, 98%, 99%, or 100% identity to a CAG promoter sequence (SEQ ID NO: 5).

>CAG promoter
(SEQ ID NO: 5)
ataacttacggtaaatggcccgcctggctgaccgcccaacgaccc
ccgcccattgacgtcaataatgacgtatgttcccatagtaacgcc
aatagggactttccattgacgtcaatgggtggagtatttacggta
aactgcccacttggcagtacatcaagtgtatcatatgccaagtac
gccccctattgacgtcaatgacggtaaatggcccgcctggcatta
tgcccagtacatgaccttatgggactttcctacttggcagtacat
ctactcgaggccacgttctgcttcactctccccatctcccccccc
tccccacccccaattttgtatttatttattttttaattattttgt
gcagcgatgggggcggggggggggggggggcgcgcgccaggcggg
gggggggggcgaggggggggggggcgaggcggagaggtgcggcgg
cagccaatcagagcggcgcgctccgaaagtttccttttatggcga
ggcggcggcggcggcggccctataaaaagcgaagcgcgcgggggg
ggagcgggatcagccaccgcggtggcggcctagagtcgacgagga
actgaaaaaccagaaagttaactg

In some embodiments, the promoter is a CMV promoter. In some embodiments, the promoter comprises SEQ ID NO: 16. In some embodiments, the promoter comprises a sequence at least 95%, 96%, 97%, 98%, 99%, or 100% identical to SEQ ID NO: 16. In some embodiments, the promoter shares at least 90%, 95%, 96%, 97%, 98%, 99%, or 100% identity to a CMV promoter sequence (SEQ ID NO: 16):

>CMV promoter
(SEQ ID NO: 16)
ctagttattaatagtaatcaattacggggtcattagttcatagcc
catatatggagttccgcgttacataacttacggtaaatggcccgc
ctggctgaccgcccaacgacccccgcccattgacgtcaataatga
cgtatgttcccatagtaacgccaatagggactttccattgacgtc
aatgggtggactatttacggtaaactgcccacttggcagtacatc
aagtgtatcatatgccaagtacgccccctattgacgtcaatgacg
gtaaatggcccgcctggcattatgcccagtacatgaccttatggg
actttcctacttggcagtacatctacgtattagtcatcgctatta
ccatggtgatgcggttttggcagtacatcaatgggcgtggatagc
ggtttgactcacggggatttccaagtctccaccccattgacgtca
atgggagtttgttttggcaccaaaatcaacgggactttccaaaat
gtcgtaacaactccgccccattgacgcaaatgggcggtaggcgtg
tacggtgggaggtctatataagcagagctctctggctaactagag
aacccactgcttactggcttategaaat

In some embodiments, the expression cassette comprises a nucleotide sequence operably linked to a polyadenylation sequence. Suitable polyadenylation sequences include bovine growth hormone polyA signal (bGHpolyA) and short poly A signal. In some embodiments, the polyadenylation sequence comprises SEQ ID NO: 21. Optionally the rAAV vectors of the disclosure comprise the Woodchuck Post-transcriptional Regulatory Element (WPRE). In some embodiments, the rAAV vector comprises a WPRE comprising SEQ ID NO: 22.

>bGHpolyA Sequence
(SEQ ID NO: 21)
cgactgtgccttctagttgccagccatctgttgtttgcccctccc
ccgtgccttccttgaccctggaaggtgccactcccactgtccttt
cctaataaaatgaggaaattgcatcgcattgtctgagtaggtgtc
attctattctggggggggggggggcaggacagcaagggggaggat
tgggaagacaatagcaggcatgctggggatgcggtgggctctatg
g
WPRE Sequence
(SEQ ID NO: 22)
tcctgttaatcaacctctggattacaaaatttgtgaaagattgac
tgatattcttaactatgttgctccttttacgctgtgtggatatgc
tgctttaatgcctctgtatcatgctattgcttcccgtacggcttt
cgttttctcctccttgtataaatcctggttgctgtctctttatga
ggagttgtggcccgttgtccgtcaacgtggcgtggtgtgctctgt
gtttgctgacgcaacccccactggctggggcattgccaccacctg
tcaactcctttctgggactttcgctttccccctccctatcgccac
ggcagaactcatcgccgcctgccttgcccgctgctggacaggggc
taggttgctgggcactgataattccgtggtgttgtcggggaagct
gacgtc

In some embodiments, the expression cassette comprises SEQ ID NO: 23. In some embodiments, the expression cassette comprises a sequence at least 90%, 95%, 96%, 97%, 98%, 99%, or 100% identical to SEQ ID NO: 23. In some embodiments, the expression cassette comprises a sequence sharing at least 90%, 95%, 96%, 97%, 98%, 99%, or 100% identity to SEQ ID NO: 23.

>Expression cassette comprising sequence
encoding GDNF
(SEQ ID NO: 23)
gcgcgctcgctcgctcactgaggccgcccgggcaaagcccgggcg
tcgggcgacctttggtcgcccggcctcagtgagcgagcgagcgcg
cagagagggagtggccaactccatcactaggggttccttgtagtt
aatgattaacggatctttacaaattcaagccaggtgatttcaaca
aattttgctgacgatttaggcgcactatcccctaaactacaaatt
agaaaatagcgttccttgacactagtctagttattaatagtaatc
aattacggggtcattagttcatagcccatatatggagttccgcgt
tacataacttacggtaaatggcccgcctggctgaccgcccaacga
cccccgcccattgacgtcaataatgacgtatgttcccatagtaac
gccaatagggactttccattgacgtcaatgggtggactatttacg
gtaaactgcccacttggcagtacatcaagtgtatcatatgccaag
tacgccccctattgacgtcaatgacggtaaatggcccgcctggca
ttatgcccagtacatgaccttatgggactttcctacttggcagta
catctacgtattagtcatcgctattaccatggtgatgcggttttg
gcagtacatcaatgggcgtggatagcggtttgactcacggggatt
tccaagtctccaccccattgacgtcaatgggagtttgttttggca
ccaaaatcaacgggactttccaaaatgtcgtaacaactccgcccc
attgacgcaaatgggcggtaggcgtgtacggtgggaggtctatat
aagcagagctctctggctaactagagaacccactgcttactggct
tatcgaaataagctttctcaggggagatctcgtttagtgaaccgt
cagatcctcactctcttccgcatcgctgtctgcgagggccagctg
ttgggctcgcggttgaggacaaactcttcgcggtctttccagtac
tcttggatcggaaacccgtcggcctccgaacggtactccgccacc
gagggacctgagcgagtccgcatcgaccggatcggaaaacctctc
gagaaaggcgtctaaccagtcacagtcgcaaggtaggctgagcac
cgtggcgggcggcagcgggtggcggtcggggttgtttctggcgga
ggtgctgctgatgatgtaattaaagtaggcggtcttgagacggcg
gatggtcgaggtgaggtgtggcaggcttgagatccagctgttggg
gtgagtactccctctcaaaagcgggcattacttctgcgctaagat
tgtcagtttccaaaaacgaggaggatttgatattcacctggcccg
atctggccatacacttgagtgacaatgacatccactttgcctttc
tctccacaggtgtccactcccaggtccaagtttaaacgccgccgc
catgaagctgtgggacgttgtggccgtgtgcctggtcctcctgca
caccgcctccgcctttccactgcctgctggaaagcggcctcctga
ggcccctgctgaagatagaagcctgggcagaagaagagccccctt
cgccctgagctctgatagcaacatgcctgaggactaccccgacca
gttcgacgacgtgatggactttatccaggccaccatcaagagact
gaaacggagccctgacaagcagatggccgtgctgcctagacggga
aagaaatagacaggccgcagctgccaaccccgagaacagcagagg
caaaggccggagaggccagaggggaaaaaacagaggatgtgtgct
gaccgccatccatctgaacgtgacagatctgggcctgggctatga
gaccaaggaagagctgatcttcagatactgcagcggcagctgcga
cgccgccgagacaacctacgacaagatcctgaagaacctgtcccg
gaatcggcggctggtgtctgacaaggtgggccaagcttgctgcag
accaattgccttcgacgatgatctgtctttcctggatgacaacct
ggtgtaccacatcctgagaaagcacagcgccaagcgctgcggctg
tatctagtcctgttaatcaacctctggattacaaaatttgtgaaa
gattgactgatattcttaactatgttgctccttttacgctgtgtg
gatatgctgctttaatgcctctgtatcatgctattgcttcccgta
cggctttcgttttctcctccttgtataaatcctggttgctgtctc
tttatgaggagttgtggcccgttgtccgtcaacgtggcgtggtgt
gctctgtgtttgctgacgcaacccccactggctggggcattgcca
ccacctgtcaactcctttctgggactttcgctttccccctcccta
tcgccacggcagaactcatcgccgcctgccttgcccgctgctgga
caggggctaggttgctgggcactgataattccgtggtgttgtcgg
ggaagctgacgtccgactgtgccttctagttgccagccatctgtt
gtttgcccctcccccgtgccttccttgaccctggaaggtgccact
cccactgtcctttcctaataaaatgaggaaattgcatcgcattgt
ctgagtaggtgtcattctattctggggggtggggtggggcaggac
agcaagggggaggattgggaagacaatagcaggcatgctggggat
gcggtgggctctatggagcgctggctagaattacctaccggcctc
caccataccttcgatattcgcgcccactctcccattaatccgcac
aagtggatgtgatgcgattgcccgctaagatagttaatcattaac
tacaaggaacccctagtgatggagttggccactccctctctgcgc
gctcgctcgctcactgaggccgggcgaccaaaggtcgcccgacgc
ccgggctttgcccgggcggcctcagtgagcgagcgagcgcgc

In some embodiments, the expression cassette comprises SEQ ID NO: 24. In some embodiments, the expression cassette comprises a sequence at least 90%, 95%, 96%, 97%, 98%, 99%, or 100% identical to SEQ ID NO: 24. In some embodiments, the expression cassette comprises a sequence shares at least 90%, 95%, 96%, 97%, 98%, 99%, or 100% identity to SEQ ID NO: 24.

>Expression cassette comprising sequence
encoding NGF
(SEQ ID NO: 24)
gcgcgctcgctcgctcactgaggccgcccgggcaaagcccgggcg
tcgggcgacctttggtcgcccggcctcagtgagcgagcgagcgcg
cagagagggagtggccaactccatcactaggggttccttgtagtt
aatgattaacggatctttacaaattcaagccaggtgatttcaaca
aattttgctgacgatttaggcgcactatcccctaaactacaaatt
agaaaatagcgttccttgacactagtctagttattaatagtaatc
aattacggggtcattagttcatagcccatatatggagttccgcgt
tacataacttacggtaaatggcccgcctggctgaccgcccaacga
cccccgcccattgacgtcaataatgacgtatgttcccatagtaac
gccaatagggactttccattgacgtcaatgggtggactatttacg
gtaaactgcccacttggcagtacatcaagtgtatcatatgccaag
tacgccccctattgacgtcaatgacggtaaatggcccgcctggca
ttatgcccagtacatgaccttatgggactttcctacttggcagta
catctacgtattagtcatcgctattaccatggtgatgcggttttg
gcagtacatcaatgggcgtggatagcggtttgactcacggggatt
tccaagtctccaccccattgacgtcaatgggagtttgttttggca
ccaaaatcaacgggactttccaaaatgtcgtaacaactccgcccc
attgacgcaaatgggcggtaggcgtgtacggtgggaggtctatat
aagcagagctctctggctaactagagaacccactgcttactggct
tatcgaaataagctttctcaggggagatctcgtttagtgaaccgt
cagatcctcactctcttccgcatcgctgtctgcgagggccagctg
ttgggctcgcggttgaggacaaactcttcgcggtctttccagtac
tcttggatcggaaacccgtcggcctccgaacggtactccgccacc
gagggacctgagcgagtccgcatcgaccggatcggaaaacctctc
gagaaaggcgtctaaccagtcacagtcgcaaggtaggctgagcac
cgtggcgggcggcagcgggtggcggtcggggttgtttctggcgga
ggtgctgctgatgatgtaattaaagtaggcggtcttgagacggcg
gatggtcgaggtgaggtgtggcaggcttgagatccagctgttggg
gtgagtactccctctcaaaagcgggcattacttctgcgctaagat
tgtcagtttccaaaaacgaggaggatttgatattcacctggcccg
atctggccatacacttgagtgacaatgacatccactttgcctttc
tctccacaggtgtccactcccaggtccaagtttaaacgccgccgc
catgagcatgctgttttacaccctgatcaccgccttcctgatcgg
catccaggccgagccccacagcgagagcaacgtgcccgctggaca
cacaatccctcaggcccactggacaaaactgcagcatagcctgga
tacagccctgagaagggccagaagcgcccctgccgccgccatcgc
cgctagagtggccggccagacaagaaatatcaccgtggacccccg
gctgttcaagaaaagaagactgcggtctcctagagtgctcttttc
aacacagcctcctcgggaagccgctgatacccaggacctggactt
cgaggtgggaggcgccgctcctttcaatagaacccacagatccaa
gagaagctctagccaccctatcttccaccggggcgagttcagcgt
gtgcgacagcgtttctgtgtggggggagataagaccaccgcaacc
gatatcaagggcaaggaagtgatggtgctgggcgaagtgaacatc
aacaacagcgtctttaagcagtacttcttcgagacaaagtgccgg
gacccaaaccccgtggacagcggctgcagaggcattgactccaag
cactggaactcctactgcaccacaacacacaccttcgtgaaggcc
ctgaccatggacggcaaacaagctgcctggcggttcatcagaatc
gacaccgcctgtgtctgtgtgctgagcagaaaggccgtgcgccgg
gcctagtcctgttaatcaacctctggattacaaaatttgtgaaag
attgactgatattcttaactatgttgctccttttacgctgtgtgg
atatgctgctttaatgcctctgtatcatgctattgcttcccgtac
ggctttcgttttctcctccttgtataaatcctggttgctgtctct
ttatgaggagttgtggcccgttgtccgtcaacgtggcgtggtgtg
ctctgtgtttgctgacgcaacccccactggctggggcattgccac
cacctgtcaactcctttctgggactttcgctttccccctccctat
ogccacggcagaactcatcgccgcctgccttgcccgctgctggac
aggggctaggttgctgggcactgataattccgtggtgttgtcggg
gaagctgacgtccgactgtgccttctagttgccagccatctgttg
tttgcccctcccccgtgccttccttgaccctggaaggtgccactc
ccactgtcctttcctaataaaatgaggaaattgcatcgcattgtc
tgagtaggtgtcattctattctggggggtggggggggcaggacag
caagggggaggattgggaagacaatagcaggcatgctggggatgc
ggtgggctctatggagcgctggctagaattacctaccggcctcca
ccataccttcgatattcgcgcccactctcccattaatccgcacaa
gtggatgtgatgcgattgcccgctaagatagttaatcattaacta
caaggaacccctagtgatggagttggccactccctctctgcgcgc
tcgctcgctcactgaggccgggcgaccaaaggtcgcccgacgccc
gggctttgcccgggcggcctcagtgagcgagcgagcgcgc

In some embodiments, the rAAV virion comprises the nucleotide sequence of SEQ ID NO: 25. In some embodiments, the rAAV virion comprises the expression cassette depicted by the plasmid in FIG. 14. In some embodiments, the expression cassette comprises a sequence at least 90%, 95%, 96%, 97%, 98%, 99%, or 100% identical to SEQ ID NO: 25. In some embodiments, the expression cassette comprises a sequence shares at least 90%, 95%, 96%, 97%, 98%, 99%, or 100% identity to SEQ ID NO: 25.

TABLE 2
pAAV-CMVE-NGF Sequence

| | |
|---|---|
| pAAV-CMVE-NGF | tcgcgcgtttcggtgatgacggtgaaaacc |
| comprising a: | tctgacacatgcagctcccggagacggtca |
| 5′ ITR | cagcttgtctgtaagcggatgccgggagca |
| CMV Promoter | gacaagcccgtcagggcgcgtcagcgggtg |
| NGF Codon- | ttggcgggtgtcggggctggcttaactatg |
| optimized | cggcatcagagcagattgtactgagagtgc |
| sequence | accatatgcggtgtgaaataccgcacagat |
| WPRE | gcgtaaggagaaaataccgcatcaggcgcc |
| 3′ ITR | attcgccattcaggctgcgcaactgttggg |
| Where each | aagggcgatcggtgcgggcctcttcgctat |
| domain above | tacgccagctggcgaaagggggatgtgctg |
| corresponds | caaggcgattaagttgggtaacgccagggt |
| in the | tttcccagtcacgacgttgtaaaacgacgg |
| sequence to | ccagtgaattgacgcgtattgggattgacc |
| a region | taacgaaaagtgaacttcataatacgtgct |
| in the | gtcccacgcacatggtagatttggacaaaa |
| same | ttgaatggagtctgatcaaccttcacaccg |
| typeface. | atctagatatcgcgcgctcgctcgctcact |
| (SEQ ID | gaggccgcccgggcaaagcccgggcgtcgg |
| NO: 25) | gcgacctttggtcgcccggcctcagtgagc |
| | gagcgagcgcgcagagagggagtggccaac |
| | tccatcactaggggttccttgtagttaatg |
| | attaacggatctttacaaattcaagccagg |
| | tgatttcaacaaattttgctgacgatttag |
| | gcgcactatcccctaaactacaaattagaa |
| | aatagcgttccttgacactagtctagttat |
| | taatagtaatcaattacggggtcattagtt |
| | catagcccatatatggagttccgcgttaca |
| | taacttacggtaaatggcccgcctggctga |
| | ccgcccaacgacccccgcccattgacgtca |
| | ataatgacgtatgttcccatagtaacgcca |
| | atagggactttccattgacgtcaatgggtg |
| | gactatttacggtaaactgcccacttggca |
| | gtacatcaagtgtatcatatgccaagtacg |
| | ccccctattgacgtcaatgacggtaaatgg |
| | cccgcctggcattatgcccagtacatgacc |
| | ttatgggactttcctacttggcagtacatc |
| | tacgtattagtcatcgctattaccatggtg |
| | atgcggttttggcagtacatcaatgggcgt |
| | ggatagcggtttgactcacggggatttcca |
| | agtctccaccccattgacgtcaatgggagt |
| | ttgttttggcaccaaaatcaacgggacttt |
| | ccaaaatgtcgtaacaactccgccccattg |
| | acgcaaatgggcggtaggcgtgtacggtgg |
| | gaggtctatataagcagagctctctggcta |
| | actagagaacccactgcttactggcttatc |
| | gaaataagctttctcaggggagatctcgtt |
| | tagtgaaccgtcagatcctcactctcttcc |
| | gcatcgctgtctgcgagggccagctgttgg |
| | gctcgcggttgaggacaaactcttcgcggt |
| | ctttccagtactcttggatcggaaacccgt |
| | cggcctccgaacggtactccgccaccgagg |
| | gacctgagcgagtccgcatcgaccggatcg |
| | gaaaacctctcgagaaaggcgtctaaccag |
| | tcacagtcgcaaggtaggctgagcaccgtg |
| | gcgggcggcagcgggtggcggtcggggttg |
| | tttctggcggaggtgctgctgatgatgtaa |
| | ttaaagtaggcggtcttgagacggcggatg |
| | gtcgaggtgaggtgtggcaggcttgagatc |
| | cagctgttggggtgagtactccctctcaaa |
| | agcgggcattacttctgcgctaagattgtc |
| | agtttccaaaaacgaggaggatttgatatt |
| | cacctggcccgatctggccatacacttgag |
| | tgacaatgacatccactttgcctttctctc |
| | cacaggtgtccactcccaggtccaagttta |
| | aacgccgccgccatgagcatgctcttttac |
| | accctgatcaccgccttcctgatcggcatc |
| | caggccgagccccacagcgagagcaacgtg |
| | cccgctggacacacaatccctcaggcccac |
| | tggacaaaactgcagcatagcctggataca |
| | gccctgagaagggccagaagcgcccctgcc |
| | gccgccatcgccgctagagtggccggccag |
| | acaagaaatatcaccgtggacccccggctg |
| | ttcaagaaaagaagactgcggtctcctaga |
| | gtgctcttttcaacacagcctcctcgggaa |
| | gccgctgatacccaggacctggacttcgag |
| | gtgggaggcgccgctcctttcaatagaacc |
| | cacagatccaagagaagctctagccaccct |
| | atcttccaccggggcgagttcagcgtgtgc |
| | gacagcgtttctgtgtgggtgggagataag |
| | accaccgcaaccgatatcaagggcaaggaa |
| | gtgatggtgctgggcgaagtgaacatcaac |
| | aacagcgtctttaagcagtacttcttcgag |
| | acaaagtgccgggacccaaaccccgtggac |
| | agcggctgcagaggcattgactccaagcac |
| | tggaactcctactgcaccacaacacacacc |
| | ttcgtgaaggccctgaccatggacggcaaa |
| | caagctgcctggcggttcatcagaatcgac |
| | accgcctgtgtctgtgtgctgagcagaaag |
| | gccgtgcgccgggcctagtcctgttaatca |
| | acctctggattacaaaatttgtgaaagatt |
| | gactgatattcttaactatgttgctccttt |
| | tacgctgtgtggatatgctgctttaatgcc |
| | tctgtatcatgctattgcttcccgtacggc |
| | tttcgttttctcctccttgtataaatcctg |
| | gttgctgtctctttatgaggagttgtggcc |
| | cgttgtccgtcaacgtggcgtggtgtgctc |
| | tgtgtttgctgacgcaacccccactggctg |
| | gggcattgccaccacctgtcaactcctttc |
| | tgggactttcgctttccccctccctatcgc |
| | cacggcagaactcatcgccgcctgccttgc |
| | ccgctgctggacaggggctaggttgctggg |
| | cactgataattccgtggtgttgtcggggaa |
| | gctgacgtccgactgtgccttctagttgcc |
| | agccatctgttgtttgcccctcccccgtgc |
| | cttccttgaccctggaaggtgccactccca |
| | ctgtcctttcctaataaaatgaggaaattg |
| | catcgcattgtctgagtaggtgtcattcta |
| | ttctggggggtggggtggggcaggacagca |
| | agggggaggattgggaagacaatagcaggc |
| | atgctggggatgcggtgggctctatggagc |
| | gctggctagaattacctaccggcctccacc |
| | ataccttcgatattcgcgcccactctccca |
| | ttaatccgcacaagtggatgtgatgcgatt |
| | gcccgctaagatagttaatcattaactaca |
| | aggaacccctagtgatggagttggccactc |
| | cctctctgcgcgctcgctcgctcactgagg |
| | ccgggcgaccaaaggtcgcccgacgcccgg |
| | gctttgcccgggcggcctcagtgagcgagc |
| | gagcgcgcgatatcgaatcgaatgcagtga |
| | tctcccaggtgcgaatcaaaaatactaggt |
| | aactagaggatttgcgacgttctaagcgtt |
| | ggtccatgtgaatcgccatccaggatccca |
| | atggcgcgccgagcttggctcgagcatggt |
| | catagctgtttcctgtgtgaaattgttatc |
| | cgctcacaattccacacaacatacgagccg |
| | gaagcataaagtgtaaagcctggggtgcct |
| | aatgagtgagctaactcacattaattgcgt |
| | tgcgctcactgcccgctttccagtcgggaa |
| | acctgtcgtgccagctgcattaatgaatcg |
| | gccaacgcgcggggagaggcggtttgcgta |
| | ttgggcgctcttccgcttcctcgctcactg |
| | actcgctgcgctcggtcgttcggctgcggc |
| | gagcggtatcagctcactcaaaggcggtaa |
| | tacggttatccacagaatcaggggataacg |
| | caggaaagaacatgtgagcaaaaggccagc |
| | aaaaggccaggaaccgtaaaaaggccgcgt |
| | tgctggcgtttttccataggctccgccccc |
| | ctgacgagcatcacaaaaatcgacgctcaa |
| | gtcagaggtggcgaaacccgacaggactat |
| | aaagataccaggcgtttccccctggaagct |
| | ccctcgtgcgctctcctgttccgaccctgc |
| | cgcttaccggatacctgtccgcctttctcc |
| | cttcgggaagcgtggcgctttctcatagct |
| | cacgctgtaggtatctcagttcggtgtagg |
| | tcgttcgctccaagctgggctgtgtgcacg |
| | aaccccccgttcagcccgaccgctgcgcct |
| | tatccggtaactatcgtcttgagtccaacc |
| | cggtaagacacgacttatcgccactggcag |
| | cagccactggtaacaggattagcagagcga |
| | ggtatgtaggcggtgctacagagttcttga |
| | agtggtggcctaactacggctacactagaa |
| | gaacagtatttggtatctgcgctctgctga |
| | agccagttaccttcggaaaaagagttggta |
| | gctcttgatccggcaaacaaaccaccgctg |
| | gtagcggtggtttttttgtttgcaagcagc |
| | agattacgcgcagaaaaaaaggatctcaag |
| | aagatcctttgatcttttctacggggtctg |
| | acgctcagtggaacgaaaactcacgttaag |
| | ggattttggtcatgagattatcaaaaagga |
| | tcttcacctagatccttttaaattaaaaat |
| | gaagttttaaatcaatctaaagtatatatg |
| | agtaaacttggtctgacagttagaaaaact |
| | catcgagcatcaaatgaaactgcaatttat |
| | tcatatcaggattatcaataccatattttt |
| | gaaaaagccgtttctgtaatgaaggagaaa |
| | actcaccgaggcagttccataggatggcaa |
| | gatcctggtatcggtctgcgattccgactc |
| | gtccaacatcaatacaacctattaatttcc |
| | cctcgtcaaaaataaggttatcaagtgaga |
| | aatcaccatgagtgacgactgaatccggtg |
| | agaatggcaaaagtttatgcatttctttcc |
| | agacttgttcaacaggccagccattacgct |
| | cgtcatcaaaatcactcgcatcaaccaaac |
| | cgttattcattcgtgattgcgcctgagcga |
| | gacgaaatacgcgatcgctgttaaaaggac |
| | aattacaaacaggaatcgaatgcaaccggc |
| | gcaggaacactgccagcgcatcaacaatat |
| | tttcacctgaatcaggatattcttctaata |
| | cctggaatgctgttttcccagggatcgcag |
| | tggtgagtaaccatgcatcatcaggagtac |
| | ggataaaatgcttgatggtcggaagaggca |
| | taaattccgtcagccagtttagtctgacca |
| | tctcatctgtaacatcattggcaacgctac |
| | ctttgccatgtttcagaaacaactctggcg |
| | catcgggcttcccatacaatcgatagattg |
| | tcgcacctgattgcccgacattatcgcgag |
| | cccatttatacccatataaatcagcatcca |
| | tgttggaatttaatcgcggcctagagcaag |
| | acgtttcccgttgaatatggctcatactct |
| | tcctttttcaatattattgaagcatttatc |
| | agggttattgtctcatgagcggatacatat |
| | ttgaatgtatttagaaaaataaacaaatag |
| | gggttccgcgcacatttccccgaaaagtgc |
| | cacctgacgtctaagaaaccattattatca |
| | tgacattaacctataaaaataggcgtatca |
| | cgaggccctttcgtc |

### Recombinant AAV Virion

In some aspects of the present invention, the subject expression cassettes are used to deliver a neurotrophic factor to at least one eye and/or lacrimal gland of a subject, e.g. to treat an ocular disorder. Accordingly, in some embodiments of the invention, the composition that provides for the expression of a neurotrophic factor in at least one eye and/or lacrimal gland of a subject is a gene delivery vector, wherein the gene delivery vector comprises the polynucleotide cassettes of the present disclosure.

In some embodiments, the gene delivery vector is an rAAV virion. In such embodiments, the subject expression cassette comprises AAV inverted terminal repeat sequences. In some embodiments, the expression cassette is flanked on the 5′ and 3′ ends by functional AAV inverted terminal repeat (ITR) sequences. By “functional AAV ITR sequences” is meant that the ITR sequences function as intended for the rescue, replication and packaging of the AAV virion. Hence, AAV ITRs for use in the gene delivery vectors of the present disclosure need not have a wild-type nucleotide sequence, and may be altered by the insertion, deletion or substitution of nucleotides or the AAV ITRs may be derived from any of several AAV serotypes, e.g. AAV1, AAV2, AAV3, AAV4, AAV5, AAV6, AAV7, AAV8, AAV9, AAV10. In some embodiments, the AAV ITR is derived from AAV1. In some embodiments, the AAV ITR is derived from AAV2. In some embodiments, the AAV ITR is derived from AAV3. In some embodiments, the AAV ITR is derived from AAV4. In some embodiments, the AAV ITR is derived from AAV5. In some embodiments, the AAV ITR is derived from AAV6. In some embodiments, the AAV ITR is derived from AAV7. In some embodiments, the AAV ITR is derived from AAV9. In some embodiments, the AAV ITR is derived from AAV1. In some embodiments, the AAV ITR is derived from AAV10. Certain rAAV virions have the wild type REP and CAP genes deleted in whole or part, but retain functional flanking ITR sequences. In some embodiments, the 5′ ITR comprises SEQ ID NO: 19. In some embodiments, the 5′ ITR comprises a sequence at least 90%, 95%, 96%, 97%, 98%, 99%, or 100% identical to SEQ ID NO: 19. In some embodiments, the 5′ ITR shares a sequence with at least 90%, 95%, 96%, 97%, 98%, 99%, or 100% identity to SEQ ID NO: 19.

>Illustrative 5′ ITR Sequence
(SEQ ID NO: 19)
gcgcgctcgctcgctcactgaggccgcccgggcaaagcccgggcg
tcgggcgacctttggtcgcccggcctcagtgagcgagcgagcgcg
cagagagggagtggccaactccatcactaggggttccttgtagtt
aatgattaac

In some embodiments, the 5′ ITR comprises SEQ ID NO: 20. In some embodiments, the 5′ ITR comprises a sequence at least 90%, 95%, 96%, 97%, 98%, 99%, or 100% identical to SEQ ID NO: 20. In some embodiments, the 5′ ITR shares a sequence with at least 90%, 95%, 96%, 97%, 98%, 99%, or 100% identity to SEQ ID NO: 20.

>Illustrative 3′ ITR Sequence
(SEQ ID NO: 20)
gttaatcattaactacaaggaacccctagtgatggagttggccac
tccctctctgcgcgctcgctcgctcactgaggccgggcgaccaaa
ggtcgcccgacgcccgggctttgcccgggcggcctcagtgagcga
gcgagcgcgc

In such embodiments, the rAAV virion comprises an AAV capsid, derived from any adeno-associated virus serotype known in the art, or prospectively discovered, including without limitation, AAVI, AAV2, AAV3, AAV4, AAV5, AAV6, AAV7, AAV8, AAV9, AAV10, etc. For example, the AAV capsid may be a wild type (or “native”) capsid. In some embodiments, the rAAV virion comprises an AAV capsid derived from AAV1. In some embodiments, the rAAV virion comprises an AAV capsid derived from AAV2. In some embodiments, the rAAV virion comprises an AAV capsid derived from AAV3. In some embodiments, the rAAV virion comprises an AAV capsid derived from AAV4. In some embodiments, the rAAV virion comprises an AAV capsid derived from AAV5. In some embodiments, the rAAV virion comprises an AAV capsid derived from AAV6. In some embodiments, the rAAV virion comprises an AAV capsid derived from AAV7. In some embodiments, the rAAV virion comprises an AAV capsid derived from AAV8. In some embodiments, the rAAV virion comprises an AAV capsid derived from AAV9. In some embodiments, the rAAV virion comprises an AAV capsid derived from AAV10. AAV capsids of particular interest include AAV2, AAV5, AAV8, and AAV9 (Table 3).

However, as with the ITRs, the capsid need not have a wild-type nucleotide sequence, but rather may be altered by the insertion, deletion or substitution of nucleotides in the VP1, VP2 or VP3 sequence, so long as the capsid is able to transduce cells of the eye and/or lacrimal glands. Put another way, the AAV capsid may be a variant AAV capsid. In some embodiments, the rAAV virion is a “pseudotyped” AAV created by using the capsid (cap) gene of one AAV and the rep gene and ITRs from a different AAV, e.g. a pseudotyped AAV2 created by using rep from AAV2 and cap from AAVI, AAV3, AAV4, AAV5, AAV6, AAV7, AAV8, or AAV9 together with a plasmid containing a vector based on AAV2. For example, the rAAV virion may be rAAV2/1, rAAV2/3, rAAV2/4, rAAV2/5, rAAV2/6, rAAV2/7, rAAV2/8, rAAV2/9, etc. In some embodiments, the rAAV is rAAV2/1. In some embodiments, the rAAV is rAAV2/3. In some embodiments, the rAAV is rAAV2/4. In some embodiments, the rAAV is rAAV2/5. In some embodiments, the rAAV is rAAV2/6. In some embodiments, the rAAV is rAAV2/7. In some embodiments, the rAAV is rAAV2/8. In some embodiments, the rAAV is rAAV2/9.

In some embodiments, the rAAV is replication defective, in that the rAAV virion cannot independently further replicate and package its genome. For example, when eye and/or lacrimal glands are transduced with rAAV virions, the gene is expressed in the transduced eye and/or lacrimal gland, however, due to the fact that the transduced eye and/or lacrimal glands lack AAV rep and cap genes and accessory function genes, the rAAV is not able to replicate.

rAAV virions of the present disclosure encapsulating the expression cassettes as described herein, can be produced using helper-free production. rAAVs are replication-deficient viruses and normally require components from a live helper virus, such as adenovirus, in a host cell for packaging of infectious rAAV virions. rAAV helper-free production systems allow the production of infectious rAAV virions without the use of a live helper virus. In the helper-free system, a host packaging cell line is co-transfected with three plasmids. A first plasmid contains adenovirus gene products (i.e., E2A, E4, and VA RNA genes) needed for the packaging of rAAV virions. A second plasmid contains the required AAV genes (i.e., REP and CAP genes). A third plasmid contains the polynucleotide sequence encoding the protein of interest and a promoter flanked by ITRs. A host packaging cell line can be, for example, AAV-293 host cells. Suitable host cells contain additional components required for packaging infectious rAAV virions that are not supplied by the plasmids. In some embodiments, the CAP genes can encode, for example, AAV capsid proteins as described herein. In some embodiments, the promoter is a promoter sequence as described herein. In some embodiments, the promoter sequence is a CAG sequence. In some embodiments, the protein of interest is a neurotrophic factor. In some embodiments, the neurotrophic factor is NGF. In some embodiments, the neurotrophic factor is GDNF.

AAV serotypes shown to infect the eye and/or lacrimal gland include AAV2, AAV5, AAV 5w8, and AAV9 (Rocha et al., supra). In some embodiments, the AAV serotype used to infect the eye and/or lacrimal gland is AAV2. In some embodiments, the AAV capsid protein comprises SEQ ID NO: 6. In some embodiments, the AAV capsid protein comprises a sequence at least 95%, 96%, 97%, 98%, 99%, or 100% identical to SEQ ID NO: 6. In some embodiments, the AAV capsid protein shares at least 95%, 98%, or 100% identity to the AAV2 VP1 protein (SEQ ID NO: 6). In some embodiments, the polynucleotide sequence encoding the AAV2 VP1 protein comprises SEQ ID NO: 7. In some embodiments, the polynucleotide sequence encoding the AAV2 VP 1 protein comprises a sequence at least 95%, 96%, 97%, 98%, 99%, or 100% identical to SEQ ID NO: 7. In some embodiments, the polynucleotide sequence encoding the AAV2 VP1 protein shares at least 95%, 98%, or 100% identity with SEQ ID NO: 7. In some embodiments, the AAV capsid protein comprises SEQ ID NO: 8. In some embodiments, the AAV capsid protein comprises a sequence at least 95%, 96%, 97%, 98%, 99%, or 100% identical to SEQ ID NO: 8. In some embodiments, the AAV capsid protein shares at least 95%, 98%, or 100% identity to the AAV2 VP3 protein (SEQ ID NO: 8). In some embodiments, the polynucleotide sequence encoding the AAV2 VP3 protein comprises SEQ ID NO: 9. In some embodiments, the polynucleotide sequence encoding the AAV capsid protein comprises a sequence at least 95%, 96%, 97%, 98%, 99%, or 100% identical to SEQ ID NO: 9. In some embodiments, the polynucleotide sequence encoding the AAV2 VP3 protein shares at least 95%, 98%, or 100% identity with SEQ ID NO: 9. In some embodiments, the AAV serotype used to infect the eye and/or lacrimal gland is AAV5. In some embodiments, the AAV capsid protein comprises SEQ ID NO: 10. In some embodiments, the AAV capsid protein comprises a sequence at least 95%, 96%, 97%, 98%, 99%, or 100% identical to SEQ ID NO: 10. In some embodiments, the AAV capsid protein shares at least 95%, 98%, or 100% identity to the AAV5 capsid protein (SEQ ID NO: 10). In some embodiments, the polynucleotide sequence encoding the AAV5 capsid protein comprises SEQ ID NO: 11. In some embodiments, the polynucleotide sequence encoding the AAV capsid protein comprises a sequence at least 95%, 96%, 97%, 98%, 99%, or 100% identical to SEQ ID NO: 11. In some embodiments, the polynucleotide sequence encoding the AAV5 capsid protein shares at least 95%, 98%, or 100% identity with SEQ ID NO: 11. In some embodiments, the AAV serotype used to infect the eye and/or lacrimal gland is AAV8. In some embodiments, the capsid protein comprises SEQ ID NO: 12. In some embodiments, the AAV capsid protein comprises a sequence at least 95%, 96%, 97%, 98%, 99%, or 100% identical to SEQ ID NO: 12. In some embodiments, the AAV capsid protein shares at least 95%, 98%, or 100% identity to the AAV8 capsid protein (SEQ ID NO: 12). In some embodiments, the polynucleotide encoding the AAV8 capsid protein comprises SEQ ID NO: 13. In some embodiments, the polynucleotide sequence encoding the AAV capsid protein comprises a sequence at least 95%, 96%, 97%, 98%, 99%, or 100% identical to SEQ ID NO: 13. In some embodiments, the polynucleotide sequence encoding the AAV8 capsid protein shares at least 95%, 98%, or 100% identity with SEQ ID NO: 13. In some embodiments, the AAV serotype used to infect the eye and/or lacrimal gland is AAV9. In some embodiments, the AAV capsid protein comprises SEQ ID NO: 14. In some embodiments, the AAV capsid protein comprises a sequence at least 95%, 96%, 97%, 98%, 99%, or 100% identical to SEQ ID NO: 14. In some embodiments, the AAV capsid protein shares at least 95%, 98%, or 100% identity to the AAV9 capsid protein (SEQ ID NO: 14). In some embodiments, the polynucleotide sequence encoding the AAV9 capsid protein comprises SEQ ID NO: 15. In some embodiments, the polynucleotide sequence encoding the AAV capsid protein comprises a sequence at least 95%, 96%, 97%, 98%, 99%, or 100% identical to SEQ ID NO: 15. In some embodiments, the polynucleotide sequence encoding the AAV9 capsid protein shares at least 95%, 98%, or 100% identity with SEQ ID NO: 15.

AAV Capsid Sequences

| | | Nucleotide |
|---|---|---|
| | Protein | (Open Reading Frame) |
| AAV2 | MAADGYLPDWLEDTLSEGIR | atggctgccgatggttatct |
| VP1 | QWWKLKPGPPPPKPAERHKD | tccagattggctcgaggaca |
| | DSRGLVLPGYKYLGPFNGLD | ctctctctgaaggaataaga |
| | KGEPVNEADAAALEHDKAYD | cagtggtggaagctcaaacc |
| | RQLDSGDNPYLKYNHADAEF | tggcccaccaccaccaaagc |
| | QERLKEDTSFGGNLGRAVFQ | ccgcagagcggcataaggac |
| | AKKRVLEPLGLVEEPVKTAP | gacagcaggggtcttgtgct |
| | GKKRPVEHSPVEPDSSSGTG | tcctgggtacaagtacctcg |
| | KAGQQPARKRLNFGQTGDAD | gacccttcaacggactcgac |
| | SVPDPQPLGQPPAAPSGLGT | aagggagagccggtcaacga |
| | NTMATGSGAPMADNNEGADG | ggcagacgccgcggccctcg |
| | VGNSSGNWHCDSTWMGDRVI | agcacgacaaagcctacgac |
| | TTSTRTWALPTYNNHLYKQI | cggcagctcgacagcggaga |
| | SSQSGASNDNHYFGYSTPWG | caacccgtacctcaagtaca |
| | YFDFNRFHCHFSPRDWQRLI | accacgccgacgcggagttt |
| | NNNWGFRPKRLNFKLFNIQV | caggagcgccttaaagaaga |
| | KEVTQNDGTTTIANNLTSTV | tacgtcttttgggggcaacc |
| | QVFTDSEYQLPYVLGSAHQG | toggacgagcagtcttccag |
| | CLPPFPADVFMVPQYGYLTL | gcgaaaaagagggttcttga |
| | NNGSQAVGRSSFYCLEYFPS | acctctgggcctggttgagg |
| | QMLRTGNNFTFSYTFEDVPF | aacctgttaagacggctccg |
| | HSSYAHSQSLDRLMNPLIDQ | ggaaaaaagaggccggtaga |
| | YLYYLSRTNTPSGTTTQSRL | gcactctcctgtggagccag |
| | QFSQAGASDIRDQSRNWLPG | actcctcctcgggaaccgga |
| | PCYRQQRVSKTSADNNNSEY | aaggcgggccagcagcctgc |
| | SWTGATKYHLNGRDSLVNPG | aagaaaaagattgaattttg |
| | PAMASHKDDEEKFFPQSGVL | gtcagactggagacgcagac |
| | IFGKQGSEKTNVDIEKVMIT | tcagtacctgacccccagcc |
| | DEEEIRTTNPVATEQYGSVS | tctcggacagccaccagcag |
| | TNLQRGNRQAATADVNTQGV | ccccctctggtctgggaact |
| | LPGMVWQDRDVYLQGPIWAK | aatacgatggctacaggcag |
| | IPHTDGHFHPSPLMGGFGLK | tggcgcaccaatggcagaca |
| | HPPPQILIKNTPVPANPSTT | ataacgagggcgccgacgga |
| | FSAAKFASFITQYSTGQVSV | gtgggtaattcctcgggaaa |
| | EIEWELQKENSKRWNPEIQY | ttggcattgcgattccacat |
| | TSNYNKSVNVDFTVDTNGVY | ggatgggcgacagagtcatc |
| | SEPRPIGTRYLTRNL | accaccagcacccgaacctg |
| | (SEQ ID NO: 6) | ggccctgcccacctacaaca |
| | | accacctctacaaacaaatt |
| | | tccagccaatcaggagcctc |
| | | gaacgacaatcactactttg |
| | | gctacagcaccccttggggg |
| | | tattttgacttcaacagatt |
| | | ccactgccacttttcaccac |
| | | gtgactggcaaagactcatc |
| | | aacaacaactggggattccg |
| | | acccaagagactcaacttca |
| | | agctctttaacattcaagtc |
| | | aaagaggtcacgcagaatga |
| | | cggtacgacgacgattgcca |
| | | ataaccttaccagcacggtt |
| | | caggtgtttactgactcgga |
| | | gtaccagctcccgtacgtcc |
| | | teggctcggcgcatcaagga |
| | | tgcctcccgccgttcccagc |
| | | agacgtcttcatggtgccac |
| | | agtatggatacctcaccctg |
| | | aacaacgggagtcaggcagt |
| | | aggacgctcttcattttact |
| | | gcctggagtactttccttct |
| | | cagatgctgcgtaccggaaa |
| | | caactttaccttcagctaca |
| | | cttttgaggacgttcctttc |
| | | cacagcagctacgctcacag |
| | | ccagagtctggaccgtctca |
| | | tgaatcctctcatcgaccag |
| | | tacctgtattacttgagcag |
| | | aacaaacactccaagtggaa |
| | | ccaccacgcagtcaaggctt |
| | | cagttttctcaggccggagc |
| | | gagtgacattcgggaccagt |
| | | ctaggaactggcttcctgga |
| | | ccctgttaccgccagcagcg |
| | | agtatcaaagacatctgcgg |
| | | ataacaacaacagtgaatac |
| | | tcgtggactggagctaccaa |
| | | gtaccacctcaatggcagag |
| | | actctctggtgaatccgggc |
| | | ccggccatggcaagccacaa |
| | | ggacgatgaagaaaagtttt |
| | | ttcctcagagcggggttctc |
| | | atctttgggaagcaaggctc |
| | | agagaaaacaaatgtggaca |
| | | ttgaaaaggtcatgattaca |
| | | gacgaagaggaaatcaggac |
| | | aaccaatcccgtggctacgg |
| | | agcagtatggttctgtatct |
| | | accaacctccagagaggcaa |
| | | cagacaagcagctaccgcag |
| | | atgtcaacacacaaggcgtt |
| | | cttccaggcatggtctggca |
| | | ggacagagatgtgtaccttc |
| | | aggggcccatctgggcaaag |
| | | attccacacacggacggaca |
| | | ttttcacccctctcccctca |
| | | tgggggatteggacttaaac |
| | | accctcctccacagattctc |
| | | atcaagaacaccccggtacc |
| | | tgcgaatccttcgaccacct |
| | | tcagtgcggcaaagtttgct |
| | | tccttcatcacacagtactc |
| | | cacgggacaggtcagcgtgg |
| | | agatcgagtgggagctgcag |
| | | aaggaaaacagcaaacgctg |
| | | gaatcccgaaattcagtaca |
| | | cttccaactacaacaagtct |
| | | gttaatgtggactttactgt |
| | | ggacactaatggcgtgtatt |
| | | cagagcctcgccccattggc |
| | | accagatacctgactcgtaa |
| | | tctgtaa |
| | | (SEQ ID NO: 7) |
| AAV2 | MATGSGAPMADNNEGADGVG | atggctacaggcagtggcgc |
| VP3 | NSSGNWHCDSTWMGDRVITT | accaatggcagacaataacg |
| | STRTWALPTYNNHLYKQISS | agggcgccgacggagtgggt |
| | QSGASNDNHYFGYSTPWGYF | aattcctcgggaaattggca |
| | DFNRFHCHFSPRDWQRLINN | ttgcgattccacatggatgg |
| | NWGFRPKRLNFKLFNIQVKE | gcgacagagtcatcaccacc |
| | VTQNDGTTTIANNLTSTVQV | agcacccgaacctgggccct |
| | FTDSEYQLPYVLGSAHQGCL | gcccacctacaacaaccacc |
| | PPFPADVFMVPQYGYLTLNN | tctacaaacaaatttccagc |
| | GSQAVGRSSFYCLEYFPSQM | caatcaggagcctcgaacga |
| | LRTGNNFTFSYTFEDVPFHS | caatcactactttggctaca |
| | SYAHSQSLDRLMNPLIDQYL | gcaccccttgggggtatttt |
| | YYLSRTNTPSGTTTQSRLQF | gacttcaacagattccactg |
| | SQAGASDIRDQSRNWLPGPC | ccacttttcaccacgtgact |
| | YRQQRVSKTSADNNNSEYSW | ggcaaagactcatcaacaac |
| | TGATKYHLNGRDSLVNPGPA | aactggggattccgacccaa |
| | MASHKDDEEKFFPQSGVLIF | gagactcaacttcaagctct |
| | GKQGSEKTNVDIEKVMITDE | ttaacattcaagtcaaagag |
| | EEIRTTNPVATEQYGSVSTN | gtcacgcagaatgacggtac |
| | LQRGNRQAATADVNTQGVLP | gacgacgattgccaataacc |
| | GMVWQDRDVYLQGPIWAKIP | ttaccagcacggttcaggtg |
| | HTDGHFHPSPLMGGFGLKHP | tttactgactcggagtacca |
| | PPQILIKNTPVPANPSTTFS | gctcccgtacgtcctcggct |
| | AAKFASFITQYSTGQVSVEI | cggcgcatcaaggatgcctc |
| | EWELQKENSKRWNPEIQYTS | ccgccgttcccagcagacgt |
| | NYNKSVNVDFTVDINGVYSE | cttcatggtgccacagtatg |
| | PRPIGTRYLTRNL | gatacctcaccctgaacaac |
| | (SEQ ID NO: 8) | gggagtcaggcagtaggacg |
| | | ctcttcattttactgcctgg |
| | | agtactttccttctcagatg |
| | | ctgcgtaccggaaacaactt |
| | | taccttcagctacacttttg |
| | | aggacgttcctttccacagc |
| | | agctacgctcacagccagag |
| | | tctggaccgtctcatgaatc |
| | | ctctcatcgaccagtacctg |
| | | tattacttgagcagaacaaa |
| | | cactccaagtggaaccacca |
| | | cgcagtcaaggcttcagttt |
| | | tctcaggccggagcgagtga |
| | | cattcgggaccagtctagga |
| | | actggcttcctggaccctgt |
| | | taccgccagcagcgagtatc |
| | | aaagacatctgcggataaca |
| | | acaacagtgaatactcgtgg |
| | | actggagctaccaagtacca |
| | | cctcaatggcagagactctc |
| | | tggtgaatccgggcccggcc |
| | | atggcaagccacaaggacga |
| | | tgaagaaaagttttttcctc |
| | | agagcggggttctcatcttt |
| | | gggaagcaaggctcagagaa |
| | | aacaaatgtggacattgaaa |
| | | aggtcatgattacagacgaa |
| | | gaggaaatcaggacaaccaa |
| | | tcccgtggctacggagcagt |
| | | atggttctgtatctaccaac |
| | | ctccagagaggcaacagaca |
| | | agcagctaccgcagatgtca |
| | | acacacaaggcgttcttcca |
| | | ggcatggtctggcaggacag |
| | | agatgtgtaccttcaggggc |
| | | ccatctgggcaaagattcca |
| | | cacacggacggacattttca |
| | | cccctctcccctcatgggtg |
| | | gatteggacttaaacaccct |
| | | cctccacagattctcatcaa |
| | | gaacaccccggtacctgcga |
| | | atccttcgaccaccttcagt |
| | | gcggcaaagtttgcttcctt |
| | | catcacacagtactccacgg |
| | | gacaggtcagcgtggagatc |
| | | gagtgggagctgcagaagga |
| | | aaacagcaaacgctggaatc |
| | | ccgaaattcagtacacttcc |
| | | aactacaacaagtctgttaa |
| | | tgtggactttactgtggaca |
| | | ctaatggcgtgtattcagag |
| | | cctcgccccattggcaccag |
| | | atacctgactcgtaatctgt |
| | | aa |
| | | (SEQ ID NO: 9) |
| AAV5 | MSFVDHPPDWLEEVGEGLRE | atgtcttttgttgatcaccc |
| | FLGLEAGPPKPKPNQQHQDQ | tccagattggttggaagaag |
| | ARGLVLPGYNYLGPGNGLDR | ttggtgaaggtcttcgcgag |
| | GEPVNRADEVAREHDISYNE | tttttgggccttgaagcggg |
| | QLEAGDNPYLKYNHADAEFQ | cccaccgaaaccaaaaccca |
| | EKLADDTSFGGNLGKAVFQA | atcagcagcatcaagatcaa |
| | KKRVLEPFGLVEEGAKTAPT | gcccgtggtcttgtgctgcc |
| | GKRIDDHFPKRKKARTEEDS | tggttataactatctcggac |
| | KPSTSSDAEAGPSGSQQLQI | ccggaaacggtctcgatcga |
| | PAQPASSLGADTMSAGGGGP | ggagagcctgtcaacagggc |
| | LGDNNQGADGVGNASGDWHC | agacgaggtcgcgcgagagc |
| | DSTWMGDRVVTKSTRTWVLP | acgacatctcgtacaacgag |
| | SYNNHQYREIKSGSVDGSNA | cagcttgaggcgggagacaa |
| | NAYFGYSTPWGYFDFNRFHS | cccctacctcaagtacaacc |
| | HWSPRDWQRLINNYWGFRPR | acgcggacgccgagtttcag |
| | SLRVKIFNIQVKEVTVQDST | gagaagctcgccgacgacac |
| | TTIANNLTSTVQVFTDDDYQ | atccttcgggggaaacctcg |
| | LPYVVGNGTEGCLPAFPPQV | gaaaggcagtctttcaggcc |
| | FTLPQYGYATLNRDNTENPT | aagaaaagggttctcgaacc |
| | ERSSFFCLEYFPSKMLRTGN | ttttggcctggttgaagagg |
| | NFEFTYNFEEVPFHSSFAPS | gtgctaagacggcccctacc |
| | QNLFKLANPLVDQYLYRFVS | ggaaagcggatagacgacca |
| | TNNTGGVQFNKNLAGRYANT | ctttccaaaaagaaagaagg |
| | YKNWFPGPMGRTQGWNLGSG | ctcggaccgaagaggactcc |
| | VNRASVSAFATTNRMELEGA | aagccttccacctcgtcaga |
| | SYQVPPQPNGMTNNLQGSNT | cgccgaagctggacccagcg |
| | YALENTMIFNSQPANPGTTA | gatcccagcagctgcaaatc |
| | TYLEGNMLITSESETQPVNR | ccagcccaaccagcctcaag |
| | VAYNVGGQMATNNQSSTTAP | tttgggagctgatacaatgt |
| | ATGTYNLQEIVPGSVWMERD | ctgcgggaggtggcggccca |
| | VYLQGPIWAKIPETGAHFHP | ttgggcgacaataaccaagg |
| | SPAMGGFGLKHPPPMMLIKN | tgccgatggagtgggcaatg |
| | TPVPGNITSFSDVPVSSFIT | cctcgggagattggcattgc |
| | QYSTGQVTVEMEWELKKENS | gattccacgtggatggggga |
| | KRWNPEIQYTNNYNDPQFVD | cagagtcgtcaccaagtcca |
| | FAPDSTGEYRTTRPIGTRYL | cccgaacctgggtgctgccc |
| | TRPL | agctacaacaaccaccagta |
| | (SEQ ID NO: 10) | ccgagagatcaaaagcggct |
| | | ccgtcgacggaagcaacgcc |
| | | aacgcctactttggatacag |
| | | caccccctgggggtactttg |
| | | actttaaccgcttccacagc |
| | | cactggagcccccgagactg |
| | | gcaaagactcatcaacaact |
| | | actggggcttcagaccccgg |
| | | tccctcagagtcaaaatctt |
| | | caacattcaagtcaaagagg |
| | | tcacggtgcaggactccacc |
| | | accaccategccaacaacct |
| | | cacctccaccgtccaagtgt |
| | | ttacggacgacgactaccag |
| | | ctgccctacgtcgtcggcaa |
| | | cgggaccgagggatgcctgc |
| | | cggccttccctccgcaggtc |
| | | tttacgctgccgcagtacgg |
| | | ttacgcgacgctgaaccgcg |
| | | acaacacagaaaatcccacc |
| | | gagaggagcagcttcttctg |
| | | cctagagtactttcccagca |
| | | agatgctgagaacgggcaac |
| | | aactttgagtttacctacaa |
| | | ctttgaggaggtgcccttcc |
| | | actccagcttcgctoccagt |
| | | cagaacctgttcaagctggc |
| | | caacccgctggtggaccagt |
| | | acttgtaccgcttcgtgagc |
| | | acaaataacactggcggagt |
| | | ccagttcaacaagaacctgg |
| | | ccgggagatacgccaacacc |
| | | tacaaaaactggttcccggg |
| | | gcccatgggccgaacccagg |
| | | gctggaacctgggctccggg |
| | | gtcaaccgcgccagtgtcag |
| | | cgccttcgccacgaccaata |
| | | ggatggagctcgagggcgcg |
| | | agttaccaggtgcccccgca |
| | | gccgaacggcatgaccaaca |
| | | acctccagggcagcaacacc |
| | | tatgccctggagaacactat |
| | | gatcttcaacagccagccgg |
| | | cgaacccgggcaccaccgcc |
| | | acgtacctcgagggcaacat |
| | | gctcatcaccagcgagagcg |
| | | agacgcagccggtgaaccgc |
| | | gtggcgtacaacgtcggggg |
| | | cagatggccaccaacaacca |
| | | gagctccaccactgcccccg |
| | | cgaccggcacgtacaacctc |
| | | caggaaatcgtgcccggcag |
| | | cgtgtggatggagagggacg |
| | | tgtacctccaaggacccatc |
| | | tgggccaagatcccagagac |
| | | gggggcgcactttcacccct |
| | | ctccggccatgggcggatte |
| | | ggactcaaacacccaccgcc |
| | | catgatgctcatcaagaaca |
| | | cgcctgtgcccggaaatatc |
| | | accagcttctcggacgtgcc |
| | | cgtcagcagcttcatcaccc |
| | | agtacagcaccgggcaggtc |
| | | accgtggagatggagtggga |
| | | gctcaagaaggaaaactcca |
| | | agaggtggaacccagagatc |
| | | cagtacacaaacaactacaa |
| | | cgacccccagtttgtggact |
| | | ttgccccggacagcaccggg |
| | | gaatacagaaccaccagacc |
| | | tatcggaacccgatacctta |
| | | cccgacccctttaa |
| | | (SEQ ID NO: 11) |
| AAV8 | MAADGYLPDWLEDNLSEGIR | atggctgccgatggttatct |
| | EWWALKPGAPKPKANQQKQD | tccagattggctcgaggaca |
| | DGRGLVLPGYKYLGPFNGLD | acctctctgagggcattcgc |
| | KGEPVNAADAAALEHDKAYD | gagtggtgggcgctgaaacc |
| | QQLQAGDNPYLRYNHADAEF | tggagccccgaagcccaaag |
| | QERLQEDTSFGGNLGRAVFQ | ccaaccagcaaaagcaggac |
| | AKKRVLEPLGLVEEGAKTAP | gacggccggggtctggtgct |
| | GKKRPVEPSPQRSPDSSTGI | tcctggctacaagtaccteg |
| | GKKGQQPARKRLNFGQTGDS | gacccttcaacggactcgac |
| | ESVPDPQPLGEPPAAPSGVG | aagggggagcccgtcaacgc |
| | PNTMAAGGGAPMADNNEGAD | ggcggacgcagcggccctcg |
| | GVGSSSGNWHCDSTWLGDRV | agcacgacaaggcctacgac |
| | ITTSTRTWALPTYNNHLYKQ | cagcagctgcaggcgggtga |
| | ISNGTSGGATNDNTYFGYST | caatccgtacctgcggtata |
| | PWGYFDFNRFHCHFSPRDWQ | accacgccgacgccgagttt |
| | RLINNNWGFRPKRLSFKLFN | caggagcgtctgcaagaaga |
| | IQVKEVTQNEGTKTIANNLT | tacgtcttttgggggcaacc |
| | STIQVFTDSEYQLPYVLGSA | tcgggcgagcagtcttccag |
| | HQGCLPPFPADVFMIPQYGY | gccaagaagcgggttctega |
| | LTLNNGSQAVGRSSFYCLEY | acctctcggtctggttgagg |
| | FPSQMLRTGNNFQFTYTFED | aaggcgctaagacggctcct |
| | VPFHSSYAHSQSLDRLMNPL | ggaaagaagagaccggtaga |
| | IDQYLYYLSRTQTTGGTANT | gccatcaccccagogttctc |
| | QTLGFSQGGPNTMANQAKNW | cagactcctctacgggcatc |
| | LPGPCYRQQRVSTTTGQNNN | ggcaagaaaggccaacagcc |
| | SNFAWTAGTKYHLNGRNSLA | cgccagaaaaagactcaatt |
| | NPGIAMATHKDDEERFFPSN | ttggtcagactggcgactca |
| | GILIFGKQNAARDNADYSDV | gagtcagttccagaccctca |
| | MLTSEEEIKTTNPVATEEYG | acctctcggagaacctccag |
| | IVADNLQQQNTAPQIGTVNS | cagcgccctctggtgtggga |
| | QGALPGMVWQNRDVYLQGPI | cctaatacaatggctgcagg |
| | WAKIPHTDGNFHPSPLMGGF | cggtggcgcaccaatggcag |
| | GLKHPPPQILIKNTPVPADP | acaataacgaaggcgccgac |
| | PTTFNQSKLNSFITQYSTGQ | ggagtgggtagttcctcggg |
| | VSVEIEWELQKENSKRWNPE | aaattggcattgcgattcca |
| | IQYTSNYYKSTSVDFAVNTE | catggctgggcgacagagtc |
| | GVYSEPRPIGTRYLTRNL | atcaccaccagcacccgaac |
| | (SEQ ID NO: 12) | ctgggccctgcccacctaca |
| | | acaaccacctctacaagcaa |
| | | atctccaacgggacatcggg |
| | | aggagccaccaacgacaaca |
| | | cctacttcggctacagcacc |
| | | ccctgggggtattttgactt |
| | | taacagattccactgccact |
| | | tttcaccacgtgactggcag |
| | | cgactcatcaacaacaactg |
| | | gggattccggcccaagagac |
| | | tcagcttcaagctcttcaac |
| | | atccaggtcaaggaggtcac |
| | | gcagaatgaaggcaccaaga |
| | | ccatcgccaataacctcacc |
| | | agcaccatccaggtgtttac |
| | | ggactcggagtaccagctgc |
| | | cgtacgttctcggctctgcc |
| | | caccagggctgcctgcctcc |
| | | gttcccggcggacgtgttca |
| | | tgattccccagtacggctac |
| | | ctaacactcaacaacggtag |
| | | tcaggccgtgggacgctcct |
| | | ccttctactgcctggaatac |
| | | tttccttcgcagatgctgag |
| | | aaccggcaacaacttccagt |
| | | ttacttacaccttcgaggac |
| | | gtgcctttccacagcagcta |
| | | cgcccacagccagagcttgg |
| | | accggctgatgaatcctctg |
| | | attgaccagtacctgtacta |
| | | cttgtctcggactcaaacaa |
| | | caggaggcacggcaaatacg |
| | | cagactctgggcttcagcca |
| | | aggtgggcctaatacaatgg |
| | | ccaatcaggcaaagaactgg |
| | | ctgccaggaccctgttaccg |
| | | ccaacaacgcgtctcaacga |
| | | caaccgggcaaaacaacaat |
| | | agcaactttgcctggactgc |
| | | tgggaccaaataccatctga |
| | | atggaagaaattcattggct |
| | | aatcctggcatcgctatggc |
| | | aacacacaaagacgacgagg |
| | | agcgtttttttcccagtaac |
| | | gggatcctgatttttggcaa |
| | | acaaaatgctgccagagaca |
| | | atgcggattacagcgatgtc |
| | | atgctcaccagcgaggaaga |
| | | aatcaaaaccactaaccctg |
| | | tggctacagaggaatacggt |
| | | atcgtggcagataacttgca |
| | | gcagcaaaacacggctcctc |
| | | aaattggaactgtcaacagc |
| | | cagggggccttacccggtat |
| | | ggtctggcagaaccgggacg |
| | | tgtacctgcagggtcccatc |
| | | tgggccaagattcctcacac |
| | | ggacggcaacttccacccgt |
| | | ctccgctgatgggcggcttt |
| | | ggcctgaaacatcctccgcc |
| | | tcagatcctgatcaagaaca |
| | | cgcctgtacctgoggatcct |
| | | ccgaccaccttcaaccagtc |
| | | aaagctgaactctttcatca |
| | | cgcaatacagcaccggacag |
| | | gtcagcgtggaaattgaatg |
| | | ggagctgcagaaggaaaaca |
| | | gcaagcgctggaaccccgag |
| | | atccagtacacctccaacta |
| | | ctacaaatctacaagtgtgg |
| | | actttgctgttaatacagaa |
| | | ggcgtgtactctgaaccccg |
| | | ccccattggcacccgttacc |
| | | tcaccegtaatctg |
| | | (SEQ ID NO: 13) |
| AAV9 | MAADGYLPDWLEDNLSEGIR | atggctgccgatggttatct |
| | EWWALKPGAPQPKANQQHQD | tccagattggctcgaggaca |
| | NARGLVLPGYKYLGPGNGLD | accttagtgaaggaattcgc |
| | KGEPVNAADAAALEHDKAYD | gagtggtgggctttgaaacc |
| | QQLKAGDNPYLKYNHADAEF | tggagcccctcaacccaagg |
| | QERLKEDTSFGGNLGRAVFQ | caaatcaacaacatcaagac |
| | AKKRLLEPLGLVEEAAKTAP | aacgctcgaggtcttgtgct |
| | GKKRPVEQSPQEPDSSAGIG | tccgggttacaaataccttg |
| | KSGAQPAKKRLNFGQTGDTE | gacccggcaacggactcgac |
| | SVPDPQPIGEPPAAPSGVGS | aagggggagccggtcaacgc |
| | LTMASGGGAPVADNNEGADG | agcagacgcggcggccctcg |
| | VGSSSGNWHCDSQWLGDRVI | agcacgacaaggcctacgac |
| | TTSTRTWALPTYNNHLYKQI | cagcagctcaaggccggaga |
| | SNSTSGGSSNDNAYFGYSTP | caacccgtacctcaagtaca |
| | WGYFDFNRFHCHFSPRDWQR | accacgccgacgccgagttc |
| | LINNNWGFRPKRLNFKLFNI | caggagcggctcaaagaaga |
| | QVKEVTDNNGVKTIANNLTS | tacgtcttttgggggcaacc |
| | TVQVFTDSDYQLPYVLGSAH | tcgggcgagcagtcttccag |
| | EGCLPPFPADVFMIPQYGYL | gccaaaaagaggcttcttga |
| | TLNDGSQAVGRSSFYCLEYF | acctcttggtctggttgagg |
| | PSQMLRTGNNFQFSYEFENV | aagcggctaagacggctcct |
| | PFHSSYAHSQSLDRLMNPLI | ggaaagaagaggcctgtaga |
| | DQYLYYLSKTINGSGQNQQT | gcagtctcctcaggaaccgg |
| | LKFSVAGPSNMAVQGRNYIP | actcctccgcgggtattggc |
| | GPSYRQQRVSTTVTQNNNSE | aaatcgggtgcacagcccgc |
| | FAWPGASSWALNGRNSLMNP | taaaaagagactcaatttcg |
| | GPAMASHKEGEDRFFPLSGS | gtcagactggcgacacagag |
| | LIFGKQGTGRDNVDADKVMI | tcagtcccagaccctcaacc |
| | TNEEEIKTTNPVATESYGQV | aatcggagaacctcccgcag |
| | ATNHQSAQAQAQTGWVQNQG | ccccctcaggtgtgggatct |
| | ILPGMVWQDRDVYLQGPIWA | cttacaatggcttcaggtgg |
| | KIPHTDGNFHPSPLMGGFGM | tggcgcaccagtggcagaca |
| | KHPPPQILIKNTPVPADPPT | ataacgaaggtgccgatgga |
| | AFNKDKLNSFITQYSTGQVS | gtgggtagttcctcgggaaa |
| | VEIEWELQKENSKRWNPEIQ | ttggcattgcgattcccaat |
| | YTSNYYKSNNVEFAVNTEGV | ggctgggggacagagtcatc |
| | YSEPRPIGTRYLTRNL | accaccagcacccgaacctg |
| | (SEQ ID NO: 14) | ggccctgcccacctacaaca |
| | | atcacctctacaagcaaatc |
| | | tccaacagcacatctggagg |
| | | atcttcaaatgacaacgcct |
| | | acttcggctacagcaccccc |
| | | tgggggtattttgacttcaa |
| | | cagattccactgccacttct |
| | | caccacgtgactggcagcga |
| | | ctcatcaacaacaactgggg |
| | | attccggcctaagcgactca |
| | | acttcaagctcttcaacatt |
| | | caggtcaaagaggttacgga |
| | | caacaatggagtcaagacca |
| | | tcgccaataaccttaccagc |
| | | acggtccaggtcttcacgga |
| | | ctcagactatcagctcccgt |
| | | acgtgctcgggtcggctcac |
| | | gagggctgcctcccgccgtt |
| | | cccagcggacgttttcatga |
| | | ttcctcagtacgggtatctg |
| | | acgcttaatgatggaagcca |
| | | ggccgtgggtcgttcgtcct |
| | | tttactgcctggaatatttc |
| | | ccgtcgcaaatgctaagaac |
| | | gggtaacaacttccagttca |
| | | gctacgagtttgagaacgta |
| | | cctttccatagcagctacgc |
| | | tcacagccaaagcctggacc |
| | | gactaatgaatccactcatc |
| | | gaccaatacttgtactatct |
| | | ctcaaagactattaacggtt |
| | | ctggacagaatcaacaaacg |
| | | ctaaaattcagtgtggccgg |
| | | acccagcaacatggctgtcc |
| | | agggaagaaactacatacct |
| | | ggacccagctaccgacaaca |
| | | acgtgtctcaaccactgtga |
| | | ctcaaaacaacaacagcgaa |
| | | tttgcttggcctggagcttc |
| | | ttcttgggctctcaatggac |
| | | gtaatagcttgatgaatcct |
| | | ggacctgctatggccagcca |
| | | caaagaaggagaggaccgtt |
| | | tctttcctttgtctggatct |
| | | ttaatttttggcaaacaagg |
| | | aactggaagagacaacgtgg |
| | | atgcggacaaagtcatgata |
| | | accaacgaagaagaaattaa |
| | | aactactaacccggtagcaa |
| | | cggagtcctatggacaagtg |
| | | gccacaaaccaccagagtgc |
| | | ccaagcacaggcgcagaccg |
| | | gctgggttcaaaaccaagga |
| | | atacttccgggtatggtttg |
| | | gcaggacagagatgtgtacc |
| | | tgcaaggacccatttgggcc |
| | | aaaattcctcacacggacgg |
| | | caactttcacccttctccgc |
| | | tgatgggagggtttggaatg |
| | | aagcaccegcctcctcagat |
| | | cctcatcaaaaacacacctg |
| | | tacctgoggatcctccaacg |
| | | gccttcaacaaggacaagct |
| | | gaactctttcatcacccagt |
| | | attctactggccaagtcagc |
| | | gtggagatcgagtgggagct |
| | | gcagaaggaaaacagcaagc |
| | | gctggaacccggagatccag |
| | | tacacttccaactattacaa |
| | | gtctaataatgttgaatttg |
| | | ctgttaatactgaaggtgta |
| | | tatagtgaaccccgccccat |
| | | tggcaccagatacctgactc |
| | | gtaatctgt |
| | | (SEQ ID NO: 15) |

Exemplary rAAVs

In some embodiments, the rAAV comprises an AAV capsid. In some embodiments, an rAAV described herein comprises an expression cassette comprising a polynucleotide encoding a neurotrophic factor. In some embodiments, the rAAV comprises an expression cassette comprising a polynucleotide encoding a neurotrophic factor operably linked to a promoter. In some embodiments, the rAAV comprises an AAV capsid, and an expression cassette comprising a polynucleotide encoding a neurotrophic factor operatively linked to a promoter. In some embodiments, the rAAV comprises an AAV capsid, and an expression cassette comprising a polynucleotide encoding a neurotrophic factor operatively linked to a CAG promoter.

In some embodiments, the rAAV comprises an AAV capsid and an expression cassette comprising a polynucleotide encoding NGF operatively linked to a promoter. In some embodiments, the rAAV comprises an AAV capsid and an expression cassette comprising a polynucleotide encoding an NGF protein comprising a sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 2, and wherein the polynucleotide is operatively linked to a promoter.

In some embodiments, the rAAV comprises an AAV capsid and an expression cassette comprising a polynucleotide encoding an NGF protein comprising a sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 17, and wherein the polynucleotide is operatively linked to a promoter. In some embodiments, the rAAV comprises an AAV capsid and an expression cassette comprising a polynucleotide encoding an NGF protein comprising an amino acid sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 1, and wherein the polynucleotide is operatively linked to a promoter. In some embodiments, the rAAV comprises an AAV capsid and an expression cassette comprising a polynucleotide encoding NGF operatively linked to a CAG promoter.

In some embodiments, the rAAV comprises an AAV capsid and an expression cassette comprising a polynucleotide encoding GDNF operatively linked to a promoter. In some embodiments, the rAAV comprises an AAV capsid and an expression cassette comprising a polynucleotide encoding an GDNF protein comprising a sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 4, and wherein the polynucleotide is operatively linked to a promoter. In some embodiments, the rAAV comprises an AAV capsid and an expression cassette comprising a polynucleotide encoding an GDNF protein comprising a sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 18, and wherein the polynucleotide is operatively linked to a promoter. In some embodiments, the rAAV comprises an AAV capsid and an expression cassette comprising a polynucleotide encoding an GDNF protein comprising an amino acid sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 3, and wherein the polynucleotide is operatively linked to a promoter. In some embodiments, the rAAV comprises an AAV capsid and an expression cassette comprising a polynucleotide encoding GDNF operatively linked to a CAG promoter.

In some embodiments, the rAAV comprises an AAV2 capsid and an expression cassette comprising a polynucleotide encoding NGF operatively linked to a promoter. In some embodiments, the rAAV comprises an AAV2 capsid and an expression cassette comprising a polynucleotide encoding an NGF protein comprising a sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 2, and wherein the polynucleotide is operatively linked to a promoter. In some embodiments, the rAAV comprises an AAV2 capsid and an expression cassette comprising a polynucleotide encoding an NGF protein comprising a sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 17, and wherein the polynucleotide is operatively linked to a promoter. In some embodiments, the rAAV comprises an AAV2 capsid and an expression cassette comprising a polynucleotide encoding an NGF protein comprising an amino acid sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 1, and wherein the polynucleotide is operatively linked to a promoter.

In some embodiments, the rAAV comprises an AAV2 capsid and an expression cassette comprising a polynucleotide encoding GDNF operatively linked to a promoter. In some embodiments, the rAAV comprises an AAV2 capsid and an expression cassette comprising a polynucleotide encoding an GDNF protein comprising a sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 4, and wherein the polynucleotide is operatively linked to a promoter. In some embodiments, the rAAV comprises an AAV2 capsid and an expression cassette comprising a polynucleotide encoding an GDNF protein comprising a sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 18, and wherein the polynucleotide is operatively linked to a promoter. In some embodiments, the rAAV comprises an AAV2 capsid and an expression cassette comprising a polynucleotide encoding an GDNF protein comprising an amino acid sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 3, and wherein the polynucleotide is operatively linked to a promoter.

In some embodiments, the rAAV comprises an AAV5 capsid and an expression cassette comprising a polynucleotide encoding NGF operatively linked to a promoter. In some embodiments, the rAAV comprises an AAV5 capsid and an expression cassette comprising a polynucleotide encoding an NGF protein comprising a sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 2, and wherein the polynucleotide is operatively linked to a promoter. In some embodiments, the rAAV comprises an AAV5 capsid and an expression cassette comprising a polynucleotide encoding an NGF protein comprising a sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 17, and wherein the polynucleotide is operatively linked to a promoter. In some embodiments, the rAAV comprises an AAV5 capsid and an expression cassette comprising a polynucleotide encoding an NGF protein comprising an amino acid sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 1, and wherein the polynucleotide is operatively linked to a promoter.

In some embodiments, the rAAV comprises an AAV5 capsid and an expression cassette comprising a polynucleotide encoding GDNF operatively linked to a promoter. In some embodiments, the rAAV comprises an AAV5 capsid and an expression cassette comprising a polynucleotide encoding an GDNF protein comprising a sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 4, and wherein the polynucleotide is operatively linked to a promoter. In some embodiments, the rAAV comprises an AAV5 capsid and an expression cassette comprising a polynucleotide encoding an GDNF protein comprising a sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 18, and wherein the polynucleotide is operatively linked to a promoter. In some embodiments, the rAAV comprises an AAV5 capsid and an expression cassette comprising a polynucleotide encoding an GDNF protein comprising an amino acid sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 3, and wherein the polynucleotide is operatively linked to a promoter.

In some embodiments, the rAAV comprises an AAV9 capsid and an expression cassette comprising a polynucleotide encoding NGF operatively linked to a promoter. In some embodiments, the rAAV comprises an AAV9 capsid and an expression cassette comprising a polynucleotide encoding an NGF protein comprising a sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 2, and wherein the polynucleotide is operatively linked to a promoter. In some embodiments, the rAAV comprises an AAV9 capsid and an expression cassette comprising a polynucleotide encoding an NGF protein comprising a sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 17, and wherein the polynucleotide is operatively linked to a promoter. In some embodiments, the rAAV comprises an AAV9 capsid and an expression cassette comprising a polynucleotide encoding an NGF protein comprising an amino acid sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 1, and wherein the polynucleotide is operatively linked to a promoter.

In some embodiments, the rAAV comprises an AAV9 capsid and an expression cassette comprising a polynucleotide encoding GDNF operatively linked to a promoter. In some embodiments, the rAAV comprises an AAV9 capsid and an expression cassette comprising a polynucleotide encoding an GDNF protein comprising a sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 4, and wherein the polynucleotide is operatively linked to a promoter. In some embodiments, the rAAV comprises an AAV9 capsid and an expression cassette comprising a polynucleotide encoding an GDNF protein comprising a sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 18, and wherein the polynucleotide is operatively linked to a promoter. In some embodiments, the rAAV comprises an AAV9 capsid and an expression cassette comprising a polynucleotide encoding an GDNF protein comprising an amino acid sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 3, and wherein the polynucleotide is operatively linked to a promoter.

In some embodiments, the rAAV comprises an AAV capsid and an expression cassette comprising a polynucleotide sequence comprising SEQ ID NO: 23.

In some embodiments, the rAAV comprises an AAV capsid and an expression cassette comprising a polynucleotide sequence comprising SEQ ID NO: 24.

In some embodiments, the rAAV comprises an AAV capsid and expression cassette comprising a polynucleotide sequence comprising SEQ ID NO: 25

### Methods of Use

Methods and compositions described herein can treat ocular conditions and reduce the associated symptoms of the ocular conditions. The terms “treatment”, “treating” and the like are used herein to generally mean obtaining a desired pharmacologic and/or physiologic effect. The effect may be prophylactic in terms of completely or partially preventing a disease or symptom thereof, e.g. reducing the likelihood that the disease or symptom thereof occurs in the subject, and/or may be therapeutic in terms of a partial or complete cure for a disease and/or adverse effect attributable to the disease. “Treatment” as used herein covers any treatment of a disease in a mammal, and includes: (a) inhibiting progress of the disease; (b) alleviating, reducing, or reducing an increase in one or more symptoms of the disease; (c) alleviating, reducing, or reducing an increase in one or more signs of the disease; (d) causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

As used herein, “administer,” “administering,” “administration” and the like refers to providing a substance (e.g., rAAV virion) to a subject in a manner that is pharmacologically useful (e.g., to treat a disease, disorder, or condition in the subject).

The methods and compositions of the present disclosure may support, maintain, and/or repair the ocular surface in a subject with an ocular condition. Ocular conditions can be, for example, neurotrophic keratitis, chemical burn of the ocular surface, corneal wound, persistent epithelial defect, dry eye disease, herpes simplex viral infection of the trigeminal nerve and/or the eye, varicella zoster virus infection of the trigeminal nerve and/or the eye, and diabetic complications of the corneal nerves. In some embodiments, the ocular condition is neurotrophic keratitis, chemical burn of the ocular surface, corneal wound, corneal ulcer, persistent epithelial defect, dry eye disease, herpes simplex viral infection of the trigeminal nerve and/or the eye, varicella zoster virus infection of the trigeminal nerve and/or the eye, or diabetic complications of the corneal nerves. In some embodiments, the ocular condition is neurotrophic keratitis. In some embodiments, the ocular condition is chemical burn of the ocular surface. In some embodiments, the ocular condition is a corneal wound. In some embodiments, the ocular condition is a corneal ulcer. In some embodiments, the ocular condition is a persistent epithelial defect. In some embodiments, the ocular condition is dry eye disease. In some embodiments, the ocular condition is herpes simplex viral infection of the trigeminal nerve and/or the eye. In some embodiments, the ocular condition is varicella zoster virus infection of the trigeminal nerve and/or the eye. In some embodiments, the ocular condition is diabetic complications of the corneal nerves.

Neurotrophic keratitis, or neurotrophic keratopathy, presents as epithelial keratopathy, ulceration, and perforation in the cornea. Neurotrophic keratitis is a degenerative disease of the corneal epithelium resulting from impaired corneal innervation. Symptoms include reduced or total loss of corneal sensitivity. Neurotrophic keratitis is diagnosed and staged using one or more of eye exam as described herein, observations of a combination of rose bengal staining of the inferior palpebral conjuctiva, tear mucus viscosity, tear break-up time, fluorescein stain, observation of nonhealing corneal defect, swelling and edematous stroma within the Descemet's membrane, corneal ulcer, corneal perforation, and corneal stromal melting (Sacchetti et al. Clin Ophthalmol.; 8: 571-579 (2014)).

In some embodiments, the disclosure provides a method of maintaining and/or repairing the ocular surface in a subject with a chemical burn of the ocular surface. Chemical burn of the ocular surface typically presents as a sudden onset of severe pain, epiphoria, and blepharospasm. Chemical burn of the ocular surface can be diagnosed through a complete eye examination. Acute periocular signs of injury include periorbital edema and erythema, deepithelialized skin, and loss of eyelashes and eyebrows. Other signs include corneal and conjunctival epithelial defects, chemosis, conjunctival inflammation, limbal ischemia, corneal cloudiness, sterile ulceration, edema, and occasionally perforation. High intraocular pressure may also be a symptom and result from damage and/or inflammation of the trabecular meshwork (Eslani et al. J Ophthalmol.; 2014:196827 (2014)).

In some embodiments, the disclosure provides a method of maintaining and/or repairing the ocular surface in a subject with a corneal wound. Corneal wounds typically present with symptoms of eye pain, tearing, sensitivity to light, and foreign body sensation. Diagnosis is performed using one or more of an eye examination, vision loss assessment, identification of corneal infiltrate or ulcer, identification of hypopyon or hyphema, evidence of penetrating eye injury, identification of irregular pupils, and extension of ocular contents. Diagnosis is performed using one or more of an eye examination, vision loss assessment, identification of corneal infiltrate or ulcer, identification of hypopyon or hyphema, evidence of penetrating eye injury, presence for foreign bodies in any ocular structures, identification of irregular pupils, and extension of ocular contents. In addition, fluorescein staining may be used to help identify any corneal abrasion (Wipperman et al. Am Fam Physician.; 87(2):114-120 (2013)).

In some embodiments, the disclosure provides a method of maintaining and/or repairing the ocular surface in a subject with a corneal ulcer. Corneal ulcer occurs when there is a failure of healing in the corneal epithelium within a normal two-week period following an injury to the ocular surface. when there is a failure of healing in the corneal epithelium within a normal two-week period following an injury to the ocular surface. Diagnosis is performed using an eye examination as described herein.

Persistent epithelial defect occurs when there is a failure of healing in the corneal epithelium within a normal two-week period following an injury to the ocular surface. Diagnosis is performed using an eye examination as described herein.

In some embodiments, the disclosure provides a method of maintaining and/or repairing the ocular surface in a subject with dry eye disease. Dry eye disease presents with blurry vision, eye irritation, a gritty or foreign body sensation, burning, tearing, photophobia, stinging, or intermittent sharp pain. Dry eye disease may be diagnosed using one or more of an eye examination as described herein, a Schirmer test or tear function index analysis to assess tear production, fluorescein staining to identify corneal epithelial defects, rose bengal staining, lissamine green staining, tear break-up time, a functional visual acuity test, and a tear meniscus assessment (Zeev et al. Clin Ophthalmol.; 8: 581-590 (2014)).

In some embodiments, the disclosure provides a method of maintaining and/or repairing the ocular surface in a subject with a herpes simplex viral infection. Herpes simplex viral infection of the trigeminal nerve and/or the eye, also known as herpes simplex keratitis, presents with symptoms that include redness, discharge, watery eyes, irritation, itching, pain, photophobia, and/or coarse granular spots that form punctuate lesions. Diagnosis is performed using one or more of an eye examination as described herein, slit-lamp examination, lissamine green staining, rose bengal staining, PCR, immunofluorescence antibody assay, and ELISA assay (Azher et al. Clin Ophthalmol. 11: 185-191 (2017)).

In some embodiments, the disclosure provides a method of maintaining and/or repairing the ocular surface in a subject with a varicella zoster virus infection. Varicella zoster virus infection of the trigeminal nerve, also herpes zoster ophthalmicus, has corneal complications that present with varying degrees of decreased vision, pain, and light sensitivity. Diagnosis includes identification of punctate epithelial keratitis, elevated dendritic plaques, granular infiltrates, or other irregularities using slit lamp examination, rose bengal staining, and fluorescein staining (Shaikh et al. Am Fam Physician. 66 (9): 1723-1730 (2002)).

In some embodiments, the disclosure provides a method of maintaining and/or repairing the ocular surface in a subject with diabetic complications of the corneal nerves. Diabetic complications of the corneal nerves present with corneal alterations that include increased corneal thickness, epithelial defects, epithelial fragility and recurrent erosions, ulcers, edema, superficial punctate keratitis, delayed and incomplete wound repair, endothelial changes, low tear secretion, dry eye syndrome, and reduced corneal sensitivity (Ljubimov et al. Vision Res.; 139: 138-152 (2017)).

As herein, the term “subject” refers to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.).

A subject can have, for example, any of the symptoms or ocular disorders described herein.

In some embodiments, the methods described herein result in one or more symptoms of the ocular condition being reduced compared to the symptoms of the ocular condition before administration of the rAAV virion. As used herein, “symptoms” include any of the diagnostic criteria or symptoms associated with a given ocular condition, including those described herein. In some embodiments, symptoms can be reduced following administration of the compositions and rAAVs of the disclosure. In some embodiments, one or more symptoms of the ocular condition are reduced compared to the symptoms of the ocular condition in an untreated control subject. In some embodiments, one or more symptoms of the ocular condition are reduced compared to the symptoms of the ocular condition in a contralateral eye. By “contralateral eye” it is meant the eye of the subject that is opposite from the eye that has been treated with a composition according to the present disclosure. The contralateral eye can be used as a control for treatment, so long as the subject suffers from bilateral disease, or in the case of a model animal, has been subjected to the experimental protocol leading up to treatment in both eyes.

In some embodiments, expression of human NGF is increased 5%, 10%, 15%, 20%, 25%, 30%, 35%, 40%, 45%, 50%, 55%, 60%, 65%, 70%, 75%, 80%, 85%, 90%, 95%, or 99% compared to expression of human NGF in an untreated subject, or in the contralateral eye of a treated subject. In some embodiments, expression of human NGF may be increased 1-fold, 2-fold, 3-fold, 4-fold, or 5-fold, 6-fold, 7-fold, 8-fold, or 9-fold compared to expression of human NGF in an untreated subject, or in the contralateral eye of a treated subject.

In some embodiments, expression of human NGF is increased for about 1 day, 2 days, 3 days, 4 days, 5 days, 6 days, 7 days, 1 week, 2 weeks, 3 weeks, 4 weeks, 1 month, 2 months, 3 months, 4 months, 5 months, 6 months, 7 months, 8 months, 9 months, 10 months, 11 months, or 1 year compared to expression of human NGF in an untreated subject, or in the contralateral eye of a treated subject.

As used herein, “approximately” or “about,” as applied to one or more values of interest, refers to a value that is similar to a stated reference value. In certain embodiments, “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1% or less in either direction (greater than or less than) of the stated reference value unless otherwise stated or otherwise evident from the context (except where such number would exceed 100% of a possible value).

In some embodiments, a subject is administered an rAAV comprising a nucleotide encoding human NGF described herein, wherein expression of human NGF is increase in the subject compared to expression of human NGF in an untreated subject, or in the contralateral eye of a treated subject.

In some embodiments, the disclosure provides a method of treating a subject with an ocular disease or disorder, comprising administering to the subject an rAAV comprising a nucleotide encoding a human NGF protein, wherein expression of human NGF is increase in the subject compared to expression of human NGF in an untreated subject, or in the contralateral eye of a treated subject.

In some embodiments, expression of human GDNF is increased 5%, 10%, 15%, 20%, 25%, 30%, 35%, 40%, 45%, 50%, 55%, 60%, 65%, 70%, 75%, 80%, 85%, 90%, 95%, or 99% compared to expression of human GDNF in an untreated subject, or in the contralateral eye of a treated subject. In some embodiments, expression of human GDNF may be increased 1-fold, 2-fold, 3-fold, 4-fold, or 5-fold, 6-fold, 7-fold, 8-fold, or 9-fold compared to expression of human GDNF in an untreated subject, or in the contralateral eye of a treated subject.

In some embodiments, expression of human GDNF is increased for about 1 day, 2 days, 3 days, 4 days, 5 days, 6 days, 7 days, 1 week, 2 weeks, 3 weeks, 4 weeks, 1 month, 2 months, 3 months, 4 months, 5 months, 6 months, 7 months, 8 months, 9 months, 10 months, 11 months, or 1 year compared to expression of human GDNF in an untreated subject, or in the contralateral eye of a treated subject.

In some embodiments, a subject is administered an rAAV comprising a nucleotide encoding human GDNF described herein, wherein expression of human GDNF is increase in the subject compared to expression of human GDNF in an untreated subject, or in the contralateral eye of a treated subject.

In some embodiments, the disclosure provides a method of treating a subject with an ocular disease or disorder, comprising administering to the subject an rAAV comprising a nucleotide encoding a human GDNF protein, wherein expression of human GDNF is increase in the subject compared to expression of human GDNF in an untreated subject, or in the contralateral eye of a treated subject.

In some embodiments, the disclosure provides rAAV virions for use in a method of treating an ocular condition in a subject in need thereof, the method comprising administering a recombinant adeno-associated virus (rAAV) virion, the rAAV virion comprising an AAV capsid and an expression cassette comprising a polynucleotide encoding a neurotrophic factor operatively linked to a promoter, to at least one eye of the subject or to at least one lacrimal gland of the eye of the subject. In some embodiments, the rAAV virion for use is administered to the lacrimal gland of a subject. In some embodiments, the rAAV virion for use comprises an expression cassette encoding an NGF protein. In some embodiments, the rAAV virion for use comprises an expression cassette encoding an GDNF protein.

In some embodiments, the disclosure provides rAAV virion for use, or adaptable for use, to treat a subject with an ocular disease, disorder, or condition. In some embodiments, the rAAV virion for use or adaptable for use comprise an AAV capsid and an expression cassette comprising a polynucleotide encoding a neurotrophic factor operatively linked to a promoter.

In some embodiments, the disclosure provides rAAV virions for use in a method of treating an ocular condition in a subject in need thereof, the method comprising administering a recombinant adeno-associated virus (rAAV) virion described herein to at least one eye of the subject or to at least one lacrimal gland of the eye of the subject.

In some embodiments, the disclosure provides rAAV virions for use in a method of treating an ocular condition in a subject in need thereof, the method comprising administering a recombinant adeno-associated virus (rAAV) virion comprising an expression cassette comprising the nucleic acid sequence of SEQ ID NO: 25 to at least one eye of the subject or to at least one lacrimal gland of the eye of the subject. In some embodiments, the disclosure provides rAAV virions for use in a method of treating an ocular condition in a subject in need thereof, the method comprising administering a recombinant adeno-associated virus (rAAV) virion comprising an expression cassette comprising the nucleic acid sequence of SEQ ID NO: 24 to at least one eye of the subject or to at least one lacrimal gland of the eye of the subject. In some embodiments, the disclosure provides rAAV virions for use in a method of treating an ocular condition in a subject in need thereof, the method comprising administering a recombinant adeno-associated virus (rAAV) virion comprising an expression cassette comprising the nucleic acid sequence of SEQ ID NO: 23 to at least one eye of the subject or to at least one lacrimal gland of the eye of the subject.

### Modes of Administration

In some aspects, the disclosure provides methods comprising administering an rAAV virion, the rAAV virion comprising an AAV capsid and an expression cassette comprising a polynucleotide encoding a neurotrophic factor operatively linked to a promoter, to the eye of the subject or to a lacrimal gland of the eye of the subject.

As noted above, the lacrimal functional unit is composed of main and accessory lacrimal glands, the ocular surface, and the interconnecting innervation. For each eye, a main lacrimal gland is situated superotemporally in the orbital within the lacrimal fossa of the frontal bone. The accessory glands, known as the Wolfring's glands and Krause's glands are located in the eyelid. In the upper eyelid there are about 2 to 5 Wolfring's glands and about forty Krause's glands. In the lower eyelid there are about 6 to 8 Krause's glands. Specific location and anatomy of the lacrimal functional unit is well-known (Conrady et al. J Ophthalmol.; 2016: 7542929 (2016)). Together, the lacrimal glands secrete tear film onto the ocular surface through lacrimal ducts. Lacrimal glands also express and secrete proteins and products necessary for corneal regeneration and promoting transparency into the tear film, such as transforming growth factor-β and retinol (Conrady et al. J Ophthalmol.; 2016: 7542929 (2016); Pan et al. Optom Vis Sci.; 95:27-31 (2018)).

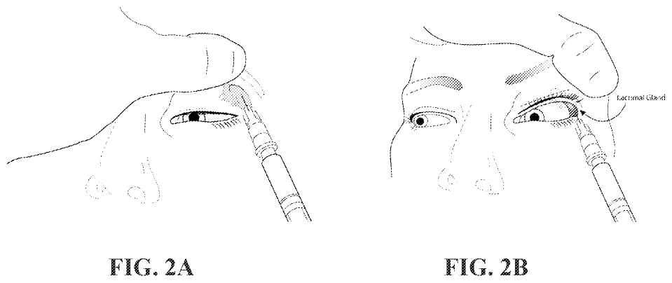

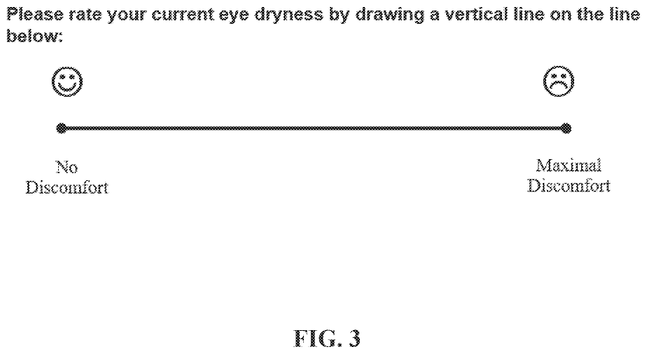

Administration of a viral vector to the lacrimal gland may be accomplished by topical administration to the ocular surface, direct injection into the lacrimal gland, and/or topical administration to the lacrimal gland. In some embodiments, the rAAV is administered to the lacrimal gland by topical administration. In some embodiments, the rAAV is administered to the lacrimal gland by direct injection. The lacrimal gland may be accessed surgically or by manipulation of the eyelid. Manipulation of the eyelid provides access to the tissue for administration topically (e.g. by lavage of the tissue with a pharmaceutical composition comprising the viral vector). Direct injection into the lacrimal gland may be done by penetrating the skin over the lacrimal gland (FIG. 2A) or by manipulating the eyelid to access the lacrimal gland (FIG. 2B). In some embodiments, the rAAV is administered to the lacrimal gland by direct injection as depicted in FIG. 2A. In some embodiments, the rAAV is administered to the lacrimal gland by manipulating the eyelid as depicted in FIG. 2B.

In some embodiments, cells within the eye and/or lacrimal gland are transduced by the rAAV virion. Cells within the eye and/or lacrimal gland are, without limitation, acinar cells, ductal cells, and/or myoepithelial cells. In some embodiments, acinar cells, ductal cells, and myoepithelial cells are transduced by the rAAV virion. In some embodiments, acinar cells are transduced by the rAAV virion. In some embodiments, ductal cells are transduced by the rAAV virion. In some embodiments, myoepithelial cells are transduced by the rAAV virion. In some embodiments, the transduced cells within the eye and/or lacrimal gland express a therapeutically effective amount of the neurotrophic factor into the tear film and optionally onto the ocular surface of the subject. In some embodiments, the transduced cells within the eye and/or lacrimal gland express an effective amount of the neurotrophic factor into the tear film and optionally onto the ocular surface of the subject. An “effective amount,” as used herein, refers to an amount or dose of an rAAV, treatment, or composition described herein that is sufficient to reduce the symptoms and or signs of an ocular condition described herein. The term “amount” as used herein refers to an absolute amount (e.g., an absolute amount of protein or rAAV particles) or concentration (e.g. a concentration of protein in a solution), whether the amount referred to in a given instance refers to an absolute amount, concentration, or both, will be clear to the skilled artisan based on the context provided herein.

Delivery of rAAV virions to the eye and/or lacrimal gland to express a transgene into tear film has been demonstrated in vivo. In one example, the main lacrimal glands of mice were directly injected with rAAV virions encoding a luciferase transgene with serotypes AAV2, AAV4, AAV5, AAV 5w8, AAV x5, AAV 9, AAV12, and bovine AAV (BAAV). AAV9, AAV 5w8, AAV5, and AAV2 each are able to transduce the lacrimal ductal and acinar cells of the lacrimal gland (Rocha et al., supra).

In some embodiments, the rAAV virion is administered to a lacrimal gland of the subject. In some embodiments, the lacrimal gland is the main lacrimal gland. In some embodiments, the lacrimal gland is any one of the Wolfring's glands or the Krause's glands of the subject.

Compositions and rAAV virions of the disclosure may be administered to the lacrimal gland of the subject by any suitable method. For example, the subject composition may be administered by direct injection to the main or accessory lacrimal glands.

Access to the lacrimal gland in human subjects can be achieved, for example, by manually elevating the upper eyelid to expose the palpebral lobe of the lacrimal gland and delivering the therapeutic agent using a syringe, e.g., with a 30 G needle.

The viral vectors of the disclosure are generally delivered to the subject as a pharmaceutical composition. Pharmaceutical compositions comprise a pharmaceutically acceptable solvent (e.g. water, etc.) and one or more excipients. In some embodiments, the pharmaceutical compositions comprise a buffer at about neutral pH (pH 5, 6, 7, 8, or 9). In some embodiments, the pharmaceutical composition comprises phosphate buffered saline (e.g. PBS at pH of about 7). The pharmaceutical compositions may comprise a pharmaceutically acceptable salt. The concentration of the salt may be selected to ensure that the pharmaceutical composition is isotonic to, or nearly isotonic to, the target tissue.

In various embodiments, the pharmaceutical compositions of the disclosure comprise about 1×10⁸ genome copies per milliliter (GC/mL), about 5×10⁸ GC/mL, about 1×10⁹ GC/mL, about 5×10⁹ GC/mL, about 1×10¹⁰ GC/mL, about 5×10¹⁰ GC/mL, about 1×10¹¹ GC/mL, about 5×10¹¹ GC/mL, about 1×10¹² GC/mL, about 5×10¹² GC/mL, about 5×10¹³ GC/mL, or about 1×10¹⁴ GC/mL of the viral vector (e.g. rAAV virion). In various embodiments, the pharmaceutical compositions of the disclosure comprise about 1×10⁸ genome copies per milliliter (GC/mL), about 5×10⁸ GC/mL to about 1×10⁹ GC/mL, about 1×10⁹ GC/mL to about 5×10⁹ GC/mL, about 5×10⁹ GC/mL to about 1×10¹⁰ GC/mL, about 1×10¹⁰ GC/mL to about 5×10¹⁰ GC/mL, about 5×10¹⁰ GC/mL to about 1×10¹¹ GC/mL, about 1×10¹¹ GC/mL to about 5×10¹¹ GC/mL, about 5×10¹¹ GC/mL to about 1×10¹² GC/mL, about 1×10¹² GC/mL to about 5×10¹² GC/mL, about 5×10¹² GC/mL to about 5×10¹³ GC/mL, or about 5×10¹³ GC/mL to about 1×10¹⁴ GC/mL of the viral vector (e.g. rAAV virion). In various further embodiments, the pharmaceutical compositions of the disclosure comprise about 5×10⁸ GC/mL to about 5×10⁹ GC/mL, about 5×10⁹ GC/mL to about 5×10¹⁰ GC/mL, about 5×10¹⁰ GC/mL to about 5×10¹¹ GC/mL, about 5×10¹¹ GC/mL to about 5×10¹² GC/mL, or about 5×10¹² GC/mL to about 1×10¹⁴ GC/mL of the viral vector (e.g. rAAV virion). In yet further embodiments, the pharmaceutical compositions of the disclosure comprise about 5×10⁸ GC/mL to about 5×10¹⁰ GC/mL, about 5×10¹⁰ GC/mL to about 5×10¹² GC/mL, or about 5×10¹² GC/mL to about 1×10¹⁴ GC/mL of the viral vector (e.g. rAAV virion).

In some embodiments, the pharmaceutical compositions of the disclosure comprise about 1×10¹² GC/mL to about 6.2×10¹² GC/mL of the viral vector (e.g., rAAV virion). In some embodiments, the pharmaceutical compositions of the disclosure comprise about 1×10¹² GC/mL or about 6.2×10¹² GC/mL of the viral vector (e.g., rAAV virion).

In some embodiments, the pharmaceutical compositions of the disclosure are administered in a total volume of about 10 μL, about 20 μL, about 30 μL, about 40 μL, about 50 μL, about 60 μL, about 70 μL, about 80 μL, about 90 μL, about 100 μL, 110 μL, about 120 μL, about 130 μL, about 140 μL, about 150 μL, about 160 μL, about 170 μL, about 180 μL, about 190 μL, or about 200 μL. In some embodiments, the pharmaceutical compositions of the disclosure are administered in a total volume of about 10 μL to about 20 μL, about 20 μL to about 30 μL, about 30 μL to about 40 μL, about 40 μL to about 50 μL, about 50 μL to about 60 μL, about 60 μL to about 70 μL, about 70 μL to about 80 μL, about 80 μL to about 90 μL, about 90 μL to about 100 μL, about 100 μL to 110 μL, 110 μL to about 120 μL, about 120 μL to about 130 μL, about 130 μL to about 140 μL, about 140 μL to about 150 μL, about 150 μL to about 160 μL, about 160 μL to about 170 μL, about 170 μL to about 180 μL, about 180 μL to about 190 μL, or about 190 μL to about 200 μL.

Genome copies per milliliter can be determined by quantitative polymerase change reaction (qPCR) using a standard curve generated with a reference sample having a known concentration of the polynucleotide genome of the virus. For AAV, the reference sample used is often the transfer plasmid used in generation of the rAAV virion but other reference samples may be used.

Alternatively, or in addition, the concentration of a viral vector can be determined by measuring the titer of the vector on a cell line. Viral titer is typically expressed as viral particles (vp) per unit volume (e.g., vp/mL). In various embodiments, the pharmaceutical compositions of the disclosure comprise about 1×10⁸ viral particles per milliliter (vp/mL), about 5×10⁸ vp/mL, about 1×10⁹ vp/mL, about 5×10⁹ vp/mL, about 1×10¹⁰ vp/mL, about 5×10¹⁰ vp/mL, about 1×10¹¹ vp/mL, about 5×10¹¹ vp/mL, about 1×10¹² vp/mL, about 5×10¹² vp/mL, about 5×10¹³ vp/mL, or about 1×10¹⁴ vp/mL of the viral vector (e.g., rAAV virion). In various further embodiments, the pharmaceutical compositions of the disclosure comprise about 1×10⁸ viral particles per milliliter (vp/mL) to about 5×10⁸ vp/mL, about 5×10⁸ vp/mL to about 1×10⁹ vp/mL, about 1×10⁹ vp/mL to about 5×10⁹ vp/mL, about 5×10⁹ vp/mL to about 1×10¹⁰ vp/mL, about 1×10¹⁰ vp/mL to about 5×10¹⁰ vp/mL, about 5×10¹⁰ vp/mL to about 1×10¹¹ vp/mL, about 1×10¹¹ vp/mL to about 5×10¹¹ vp/mL, about 5× 10¹¹ vp/mL to about 1×10¹² vp/mL, about 1×10¹² vp/mL to about 5×10¹² vp/mL, about 5×10¹² vp/mL to about 5×10¹³ vp/mL, or about 5×10¹³ vp/mL to about 1×10¹⁴ vp/mL of the viral vector (e.g., rAAV virion).

A variety of tests are available to evaluate ocular conditions in a subject before, during, and after treatment with any of the methods or compositions disclosed herein. In some of the embodiments disclosed herein, the effective treatment of the subject is indicated by one or more of the tests that can be, for example, a) Eye Dryness score test on a visual analog scale, b) Schirmer's test, c) Corneal Fluorescein Staining test, and d) Ocular Surface Disease Index test. Tests to evaluate the signs and symptoms of an ocular condition may be administered under standardized or reproducible conditions in order to obtain a subject's test score. Conditions include exposing the subject to an environment artificially created to adversely challenge the subject or where the environment (temperature, humidity, air flow) is monitored and carefully controlled.

The next sections provide further details on the Eye Dryness Score test on a visual analog scale, Schirmer's test, Corneal Staining test, and Ocular Surface Disease Index test.

### Eye Dryness Score Test

An Eye Dryness Score test on a visual analog scale may be used to evaluate ocular conditions, tearing levels, or ocular discomfort in a subject. The test involves the use of a visual analog scale and comprises a 100 mm horizontal line where one endpoint at 0 is labeled “no discomfort” and the other endpoint at 100 is labeled “maximal discomfort.” FIG. 3 shows an example of the visual analog scale (not shown at actual scale). The subject is asked to rate their ocular symptoms due to eye dryness by placing a vertical mark on the horizontal line to indicate their level of discomfort. The Eye Dryness score is then obtained by identifying where the subject's response is on the 100 mm scale.

The Eye Dryness score, measured in mm, can be used to evaluate the severity of the ocular symptom, and the effectiveness of a particular treatment of a subject. This test has the advantage of being administered to the subject as frequently as every 5 minutes, which allows the test administrator to closely monitor the changes in the subject's symptoms over time. Higher numbers indicate more discomfort for an ocular symptom compared to lower numbers, which indicate relatively lower levels of discomfort. A decrease in the Eye Dryness score is evidence that the treatment is effective in treating an ocular condition, increasing tear production, or improving ocular discomfort. Reduction in Eye Dryness scores over time is evidence of a reduction in or alleviation of the ocular symptom and generally indicates an improvement in the subject's condition.

In some of the embodiments disclosed herein, effective treatment is indicated by a statistically significant decrease in the subject's Eye Dryness score, and wherein the statistically significant decrease in the subject's Eye Dryness score is determined after administration to the subject of the first dose, or the optionally one or more subsequent doses, of the rAAV virion, and the first dose, or the optionally one or more subsequent doses, of the treatment that increases tear production, wherein the subject's Eye Dryness score is compared to a) an Eye Dryness score of the subject prior to administration of the first dose of the rAAV virion, and the first dose of the treatment that increases tear production; b) an Eye Dryness score of a subject administered a control; c) an Eye Dryness score of a subject administered a comparator compound; or d) an Eye Dryness score in a contralateral eye.

As used herein, the term “statistically significant” refers to a method of analysis selected by a person of skill in the art based upon study design and the data type generated to assess the difference observed between two or more groups, wherein the analysis determines if the difference is not random or due strictly to chance.

In some of the embodiments disclosed herein, the subject's Eye Dryness score is compared to an Eye Dryness score of the subject prior to administration of the first dose of the rAAV virion, and the first dose of the treatment that increases tear production.

In some of the embodiments disclosed herein, the subject's Eye Dryness score is compared to an Eye Dryness score of a subject administered a control.

In some of the embodiments disclosed herein, the subject's Eye Dryness score is compared to an Eye Dryness score of a subject administered a comparator compound.

In some of the embodiments disclosed herein, the subject's Eye Dryness score is compared to an Eye Dryness score in a contralateral eye.

In some embodiments, a statistically significant decrease in a subject's Eye Dryness score is observed in a subject administered an rAAV virion described herein compared to a control. In some of the embodiments described herein, the statistically significant decrease in the subject's Eye Dryness Score is at least 5%, 10%, 15%, 20%, 25%, 50%, 75%, 80%, 90%, 95%, 100%, 125%, 150%, 200%, 250%, 300%, or 350%. In some embodiments, a statistically significant decrease in a subject's Eye Dryness score is observed in a subject administered an rAAV virion described herein compared to a control, wherein the statistically significant decrease in the subject's Eye Dryness Score is at least 5%, 10%, 15%, 20%, 25%, 50%, 75%, 80%, 90%, 95%, 100%, 125%, 150%, 200%, 250%, 300%, or 350%.

In some of the embodiments described herein, the statistically significant decrease in the subject's Eye Dryness score is at least 3 mm, at least 5 mm, at least 10 mm, at least 15 mm, at least 20 mm, at least 25 mm, at least 30 mm, at least 35 mm, at least 40 mm, at least 45 mm, or at least 50 mm. In some embodiments, a statistically significant decrease in a subject's Eye Dryness score is observed in a subject administered an rAAV virion described herein compared to a control, wherein the statistically significant decrease in the subject's Eye Dryness Score is at least 3 mm, at least 5 mm, at least 10 mm, at least 15 mm, at least 20 mm, at least 25 mm, at least 30 mm, at least 35 mm, at least 40 mm, at least 45 mm, or at least 50 mm.

In some of the embodiments described herein, the statistically significant decrease in the subject's Eye Dryness score is between 3 mm and 10 mm, between 3 mm and 20 mm, between 3 mm and 25 mm, between 3 mm and 30 mm, between 3 mm and 35 mm, between 3 mm and 40 mm, between 3 mm and 45 mm, between 3 mm and 50 mm, between 5 mm and 10 mm, between 5 mm and 20 mm, between 5 mm and 25 mm, between 5 mm and 30 mm, between 5 mm and 35 mm, between 5 mm and 40 mm, between 5 mm and 45 mm, between 5 mm and 50 mm, between 10 mm and 15 mm, between 10 mm and 20 mm, between 10 mm and 25 mm, between 10 mm and 30 mm, between 10 mm and 35 mm, between 10 mm and 40 mm, between 10 mm and 45 mm, between 10 mm and 50 mm, between 15 mm and 20 mm, between 20 mm and 30 mm, between 25 mm and 35 mm, between 30 mm and 40 mm, between 30 mm and 45 mm, or between 30 mm and 50 mm.

In some of the embodiments described herein, the statistically significant decrease in the subject's Eye Dryness Score is characterized by a p value of 0.05 or less, 0.01 or less, 0.005 or less, or 0.001 or less. In some of the embodiments described herein, the statistically significant decrease in the subject's Eye Dryness Score is characterized by a p value of 0.05 or less. In some of the embodiments described herein, the statistically significant decrease in the subject's Eye Dryness Score is characterized by a p value of 0.01 or less.

In some of the embodiments described herein, the statistically significant decrease in the subject's Eye Dryness Score is within 1 day, within 2 days, within 3 days, within 4 days, within 1 week, within 2 weeks, within 3 weeks, within 4 weeks, within 1 month, within 2 months, within 3 months, within 4 months, within 5 months or within 6 months after treatment with any of the methods or compositions disclosed.

In some embodiments, the decrease in a subject's Eye Dryness Score persists for about 1 day, 2 days, 3 days, 4 days, 5 days, 6 days, 7 days, 1 week, 2 weeks, 3 weeks, 4 weeks, 1 month, 2 months, 3 months, 4 months, 5 months, 6 months, 9 months, or 1 year after treatment with any of the methods or compositions disclosed herein.

In some of the embodiments described herein, wherein the statistically significant decrease in the subject's Eye Dryness score is based on the subject's Eye Dryness score determined after administering the first dose of the rAAV virion.

In some of the embodiments described herein, the statistically significant decrease is based on the subject's Eye Dryness score determined after treatment with any of the methods or compositions disclosed herein.

### Schirmer Score Test

Ocular conditions may affect tear volume and tear production. A Schirmer's test may be used to evaluate tear production and assess the severity of dry eye disease, insufficient tearing, or ocular discomfort in a subject. The test measures the amount of tears produced in each eye. The test typically involves first placing an anesthetic into one or both of the subject's eyes. These drops prevent the eyes from watering in reaction to the test strips. Then, the test administrator places a piece of filter paper inside one or both lower eyelids and the person closes their eyes. After 5 minutes, the test administrator removes the filter paper and assesses how far the tears have travelled on the paper. The Schirmer's test may be administered to one or both eyes.

In general, the smaller the amount of moisture on the paper, the fewer tears that person has produced. In healthy eyes, each strip of paper typically contains more than 10 millimeters of moisture. A Schirmer's score of less than 10 millimeters of moisture may indicate one or more of the following conditions including the ocular conditions described herein, abnormally low tearing, or ocular discomfort.

A Schirmer's test may be used to evaluate the effectiveness of a particular treatment in a subject and may be administered multiple times to monitor any change in the severity of the subject's symptoms over a period of time. An increase in Schirmer's scores over time in a subject being treated for ocular conditions described herein, insufficient tearing, or ocular discomfort is evidence for an increase in tear volume or tear production and generally indicates improvement in the subject's condition. An increase in the Schirmer's score over time is evidence that treatment is effective in treating ocular conditions described herein, increasing tear production, or improving ocular discomfort.

In some of the embodiments disclosed herein, effective treatment is indicated by a statistically significant increase in the subject's Schirmer's score, and wherein the statistically significant increase in the subject's Schirmer's score is determined after treatment with any of the methods or compositions disclosed herein, wherein the subject's Schirmer's score is compared to a) a Schirmer's score of the subject prior to administration of the first dose of the rAAV virion, and the first dose of the treatment that increases tear production; b) a Schirmer's score of a subject administered a control; c) a Schirmer's score of a subject administered a comparator compound; or d) a contralateral eye.

In some of the embodiments disclosed herein, the subject's Schirmer's score is compared to a Schirmer's score of the subject prior to administration of a pharmaceutical composition described herein. In some of the embodiments disclosed herein, the subject's Schirmer's score is compared to a Schirmer's score of a subject administered a control. In some of the embodiments disclosed herein, the subject's Schirmer's score is compared to a Schirmer's score of a subject administered a comparator compound. In some of the embodiments disclosed herein, the subject's Schirmer's score is compared to a contralateral eye.

In some embodiments, a statistically significant increase in a subject's Schirmer's score is observed in a subject administered an rAAV virion described herein compared to a control.

In some of the embodiments described herein, the statistically significant increase in the subject's Schirmer's score is at least 5%, 10%, 15%, 20%, 25%, 50%, 75%, 80%, 90%, 95%, 100%, 125%, 150%, 200%, 250%, 300%, or 350%. In some of the embodiments described herein, the increase in the subject's Schirmer's score is at least 100%, 200%, or 300%. In some embodiments, a statistically significant increase in a subject's Schirmer's score is observed in a subject administered an rAAV virion described herein compared to a control, wherein the statistically significant increase in the subject's Schirmer's Score is at least 5%, 10%, 15%, 20%, 25%, 50%, 75%, 80%, 90%, 95%, 100%, 125%, 150%, 200%, 250%, 300%, or 350%.

In some of the embodiments described herein, the statistically significant increase in the subject's Schirmer's score is at least 3 mm, at least 5 mm, at least 10 mm, at least 15 mm, at least 20 mm, at least 25 mm, at least 30 mm, at least 35 mm, at least 40 mm, at least 45 mm, or at least 50 mm.

In some of the embodiments described herein, the statistically significant increase in the subject's Schirmer's score is between 3 mm and 5 mm, between 3 mm and 10 mm, between 3 mm and 15 mm, between 3 mm and 20 mm, between 3 mm and 25 mm, between 3 mm and 30 mm, between 5 mm and 10 mm, between 5 mm and 15 mm, between 5 mm and 20 mm, between 5 mm and 25 mm, between 5 mm and 30 mm, between 10 mm and 15 mm, between 10 mm and 20 mm, between 10 mm and 25 mm, between 10 mm and 30 mm, 15 mm and 20 mm, between 15 mm and 25 mm, between 15 mm and 30 mm, between 20 mm and 25 mm, or between 20 mm and 30 mm.

In some embodiments, a statistically significant increase in a subject's Schirmer score is observed in a subject administered an rAAV virion described herein compared to a control, wherein the statistically significant increase in the subject's Schirmer Score is at least 3 mm, at least 5 mm, at least 10 mm, at least 15 mm, at least 20 mm, at least 25 mm, at least 30 mm, at least 35 mm, at least 40 mm, at least 45 mm, or at least 50 mm.

In some of the embodiments described herein, the statistically significant increase in the subject's Schirmer's score is characterized by a p value of 0.05 or less, 0.01 or less, 0.005 or less, or 0.001 or less.

In some of the embodiments described herein, the statistically significant increase in the subject's Schirmer's score is within 1 day, within 2 days, within 3 days, within 4 days, within 1 week, within 2 weeks, within 3 weeks, within 4 weeks, within 1 month, within 2 months, within 3 months, within 4 months, within 5 months or within 6 months after treatment according to the methods or compositions disclosed herein. In some of the embodiments described herein, the statistically significant increase in the subject's Schirmer's score is within 1 minute, 2 minutes, 5 minutes, when measured acutely, or withing 1 day, within 2 days, within 3 days, within 4 days, within 1 week, within 2 weeks, within 3 weeks, within 4 weeks, within 1 month, within 2 months, within 3 months, within 4 months, within 5 months or within 6 months when measured chronically after treatment according to the methods or compositions disclosed herein.

In some embodiments, the increase in a subject's Schirmer's score persists for 1 minute, 2 minutes, 3 minutes, or at least 1 day, 2 days, 3 days, 4 days, 5 days, 6 days, 7 days, 1 week, 2 weeks, 3 weeks, 4 weeks, 1 month, 2 months, 3 months, 4 months, 5 months, 6 months, 9 months, or 1 year after treatment with any of the methods or compositions disclosed herein.

In some of the embodiments described herein, wherein the statistically significant increase in the subject's Schirmer's score is based on the subject's Schirmer's score determined after treatment according to the methods or compositions disclosed herein.

In some of the embodiments described herein, the statistically significant increase is based on the subject's Schirmer's score determined after treatment according to any of the methods or compositions disclosed herein.

### Corneal Staining Test

Corneal surface changes are associated with insufficient tear flow and excessive dryness, as well as the ocular conditions described herein and ocular discomfort. Corneal surface changes may include disruption of the mucin coating protecting the surface epithelial cells, and/or damage to the epithelial cell walls.

Corneal Staining, including fluorescein staining, lissamine green staining, and rose bengal staining, tests are diagnostic tests for determining corneal surface health and can indicate areas of damage on the corneal surface. The normal corneal surface does not take up water-soluble dyes instilled into the tear film. However, damaged epithelial cells allow water-soluble dyes to diffuse into the surface cells. The dyes, which stain damaged epithelial cells, may be visualized on the corneal surface indicating damage on the corneal surface.

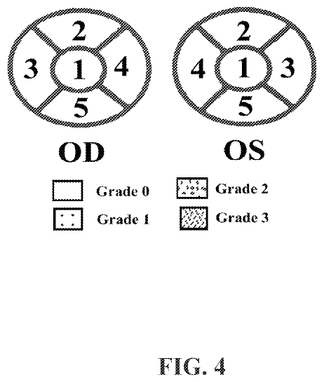

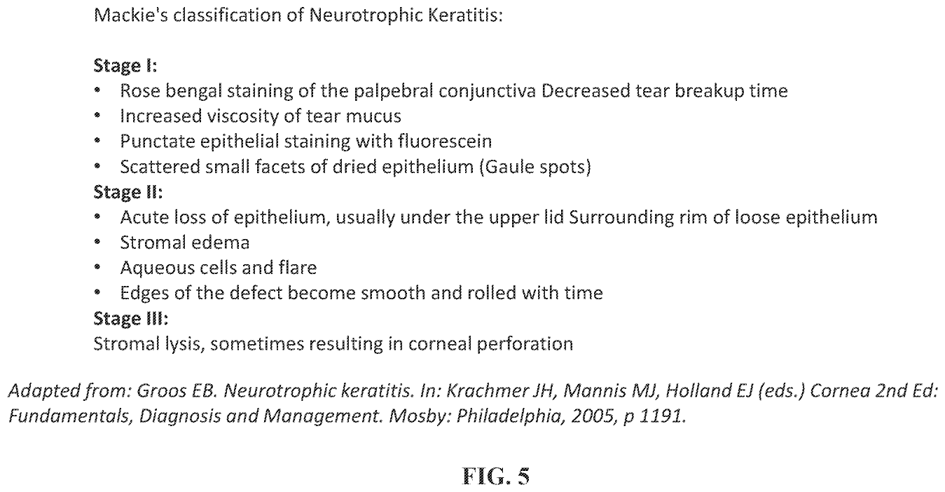

To administer a Corneal Staining test, a staining dye is applied to one or both eyes. The dye is allowed to penetrate and stain the area between surface cells. Devitalized cells and strands of devitalized surface tissue (filaments) can be visualized with this test. A test administrator then uses a corneal surface scoring system developed to rate the severity of damage observed. This scoring system is useful for treatment of an ocular condition over time. FIG. 4 depicts the NEI/Industry Workshop Scale used in the scoring system. Other equivalent standardized scoring systems may be used. A test administrator grades the areas of the corneal surface that are damaged and calculates the corneal score reflecting the severity of damage to the corneal surface.

A test administrator may use the corneal score to evaluate the effectiveness of a particular treatment in a subject. The test may be administered multiple times to monitor any change in the severity of the subject's ocular surface over a period of time. In general, higher numbers indicate more damage to the corneal surface compared to lower numbers, which indicate lower levels of damage to the corneal surface. Reduction in corneal scores over time is evidence of a reduction in the damage to corneal surface. The decrease in corneal scores generally indicates and improvement in the subject's condition. The decrease in corneal scores over time is also evidence that the treatment is effective in treating dry eye disease, increasing tear production, or improving ocular discomfort.

In some of the embodiments disclosed herein, effective treatment is indicated by a statistically significant decrease in the subject's corneal score, and wherein the statistically significant decrease in the subject's corneal score is determined after administration to the subject of the first dose, or the optionally one or more subsequent doses, of the compositions described herein, wherein the subject's corneal score is compared to a) a corneal score of the subject prior to administration of the first dose of the rAAV virion, and the first dose of the treatment that increases tear production; b) a corneal score of an subject administered a control; c) a corneal score of an subject administered a comparator compound; or d) a contralateral eye.

In some of the embodiments disclosed herein, the subject's corneal score is compared to a corneal score of the subject prior to administration of the pharmaceutical composition according to the methods described herein. In some of the embodiments disclosed herein, the subject's corneal score is compared to a corneal score of a subject administered a control. In some of the embodiments disclosed herein, the subject's corneal score is compared to a corneal score of a subject administered a comparator compound. In some of the embodiments disclosed herein, the subject's corneal score is compared to a contralateral eye.

In some of the embodiments disclosed herein, the subject's corneal score may be used to measure a definition of corneal healing defined as less than 0.5-mm fluorescein staining or no (zero) fluorescein staining (Bonini et al., Ophthalmology. 125:1332-1343 (2018)).

In some embodiments, a statistically significant decrease in a subject's corneal healing score is observed in a subject administered an rAAV virion described herein compared to a control.

In some of the embodiments described herein, the statistically significant decrease in the subject's corneal healing score is at least 5%, 10%, 15%, 20%, 25%, 50%, 75%, 80%, 90%, 95%, 100%, 125%, 150%, 200%, 250%, 300%, or 350%.

In some embodiments, a statistically significant decrease in a subject's corneal healing score is observed in a subject administered an rAAV virion described herein compared to a control, wherein the statistically significant decrease in the subject's corneal healing score is at least 5%, 10%, 15%, 20%, 25%, 50%, 75%, 80%, 90%, 95%, 100%, 125%, 150%, 200%, 250%, 300%, or 350%.

In some of the embodiments described herein, the statistically significant decrease in the subject's corneal score is characterized by a p value of 0.05 or less, 0.01 or less, 0.005 or less, or 0.001 or less.

In some of the embodiments described herein, the statistically significant decrease in the subject's corneal score is within 1 day, within 2 days, within 3 days, within 4 days, within 1 week, within 2 weeks, within 3 weeks, within 4 weeks, within 1 month, within 2 months, within 3 months, within 4 months, within 5 months or within 6 months after treatment according to the methods or compositions disclosed herein.

In some embodiments, the decrease in a subject's corneal score persists for at least 1 day, 2 days, 3 days, 4 days, 5 days, 6 days, 7 days, 1 week, 2 weeks, 3 weeks, 4 weeks, 1 month, 2 months, 3 months, 4 months, 5 months, 6 months, 9 months, or 1 year after treatment with any of the methods or compositions disclosed herein.

In some of the embodiments described herein, the statistically significant decrease is based on the subject's corneal score determined after administering one or more subsequent doses of the rAAV virion, and optionally the first or the one or more subsequent doses of the treatment that increases tear production.

### Ocular Surface Disease Index Test

Subjects treated for ocular conditions and associated symptoms can provide important information used to diagnose their condition and determine the severity of symptoms through questionnaires. A well-designed questionnaire can be validated for reproducibility and should consist of relevant questions that elicit responsive answers. One example of a questionnaire is the Ocular Surface Disease Index (OSDI), a 12-question survey for subjects with an ocular condition that's been shown to be a reliable and valid instrument for directly assessing symptom frequency. The ocular symptoms that are evaluated include, but are not limited to, burning/stinging, itching, foreign body sensation, eye discomfort, eye dryness, photophobia, and pain. Most people are familiar with questionnaires and understand that they are an efficient way for a health provider to gather information. Questionnaires also reduce bias, as there are no verbal or visual cues to inadvertently influence the respondent. The test administrator collects the response from the subject and calculates an OSDI based on the subject's answers to the questions.

The OSDI score can be used to evaluate the severity of the ocular symptom, and the effectiveness of a particular treatment of a subject. Higher numbers indicate a higher severity of an ocular condition. Reduction in OSDI scores over time is evidence of a reduction in or alleviation of the ocular symptoms and generally indicates an improvement in the subject's condition. A decrease in the OSDI score is also evidence that the treatment is effective in treating an ocular conditions and reducing symptoms.

In some of the embodiments disclosed herein, effective treatment is indicated by a statistically significant decrease in the subject's OSDI score, and wherein the statistically significant decrease in the subject's OSDI score is determined after administration to the subject of the first dose, or the optionally one or more subsequent doses, of the rAAV virion, and the first dose, or the optionally one or more subsequent doses, of the treatment that increases tear production, wherein the subject's OSDI score is compared to a) an OSDI score of the subject prior to administration of the first dose of the rAAV virion, and the first dose of the treatment that increases tear production; b) an OSDI score of an subject administered a control; c) an OSDI score of a subject administered a comparator compound; or d) a contralateral eye.

In some of the embodiments disclosed herein, the subject's OSDI score is compared to an OSDI score of the subject prior to treatment according to the disclosed methods described herein. In some of the embodiments disclosed herein, the subject's OSDI score is compared to an OSDI score of a subject administered a control. In some of the embodiments disclosed herein, the subject's OSDI score is compared to an OSDI score of a subject administered a comparator compound. In some of the embodiments disclosed herein, the subject's OSDI score is compared to an OSDI score of a contralateral eye.

In some embodiments, a statistically significant decrease in a subject's OSDI score is observed in a subject administered an rAAV virion described herein compared to a control.

In some of the embodiments described herein, the statistically significant decrease in the subject's OSDI score is at least 5%, 10%, 15%, 20%, 25%, 50%, 75%, 80%, 90%, 95%, 100%, 125%, 150%, 200%, 250%, 300%, or 350%. In some embodiments, a statistically significant decrease in a subject's OSDI score is observed in a subject administered an rAAV virion described herein compared to a control, wherein the statistically significant decrease in the subject's OSDI Score is at least 5%, 10%, 15%, 20%, 25%, 50%, 75%, 80%, 90%, 95%, 100%, 125%, 150%, 200%, 250%, 300%, or 350%.

In some of the embodiments described herein, the statistically significant decrease in the subject's OSDI score is characterized by a p value of 0.05 or less, 0.01 or less, 0.005 or less, or 0.001 or less.

In some of the embodiments described herein, the statistically significant decrease in the subject's OSDI score is within 1 day, within 2 days, within 3 days, within 4 days, within 1 week, within 2 weeks, within 3 weeks, within 4 weeks, within 1 month, within 2 months, within 3 months, within 4 months, within 5 months or within 6 months after treatment according to the methods or compositions disclosed herein.

In some embodiments, the decrease in a subject's OSDI score persists for at least 1 day, 2 days, 3 days, 4 days, 5 days, 6 days, 7 days, 1 week, 2 weeks, 3 weeks, 4 weeks, 1 month, 2 months, 3 months, 4 months, 5 months, 6 months, 9 months, or 1 year after treatment with any of the methods or compositions disclosed herein.

In some of the embodiments described herein, wherein the statistically significant decrease in the subject's OSDI score is based on the subject's OSDI score determined after administering the first dose of the rAAV virion.

In some of the embodiments described herein, the statistically significant decrease is based on the subject's OSDI score determined after treatment according to the methods or compositions disclosed herein.

### Maintenance Of Effective Treatment Over Time

The present disclosure may provide for effective treatment over a period of time where a statistically significant improvement in a subject's score is maintained. The term “maintained” as used in the present disclosure and as it relates to the maintenance of a statistically significant improvement in a subject's score (EDS, Schirmer, corneal staining, or OSDI) refers to the statistically significant improvement not diminishing below a certain threshold over time. A statistically significant improvement can be maintained even if, at a later point in time, the subject's score changes. An improvement after treatment according to the disclosed methods, can be maintained without additional dosing or after one or more subsequent doses.

For example, a corneal staining score of 0.5-mm or 0-mm could be defined as corneal healing and represent a significant improvement at 30 or 60 days after initiation of therapy (Bonini et al., Ophthalmology. 125:1332-1343 (2018)).

For example, an Eye Dryness score being “maintained within 10% means” that the decrease in subject's Eye Dryness score does not diminish by more than 10% during the specified time. A further improvement in the subject's Eye Dryness score would also be considered maintenance of the statistically significant improvement (e.g., if the Eye Dryness score further improved by 15% during the specified time, this would be considered “maintained within 10%”).

In another example, if there is a statistically significant decrease (improvement) in a subject's Eye Dryness score 30 days after treatment according to the methods described herein, and at a later point, the subject's score is the same or lesser (indicating a benefit to the subject), then the statistically significant improvement is said to be maintained. Alternatively, if at a later point, the subject's Eye Dryness score is greater than the Eye Dryness score 30 days after treatment according to the methods described herein, the subject may still be receiving a therapeutic benefit and the later determined score may still be a statistically significant improvement compared to prior administration of the first dose, or their pre-treatment baseline score.

In another example, if there is a statistically significant decrease (improvement) in a subject's Eye Dryness score 60 days after treatment according to the methods described herein, and at a later point, the subject's score is the same or lesser (indicating a benefit to the subject), then the statistically significant improvement is said to be maintained. Alternatively, if at a later point, the subject's Eye Dryness score is greater than the Eye Dryness score 60 days after treatment according to the methods described herein, the subject may still be receiving a therapeutic benefit and the later determined score may still be a statistically significant improvement compared to prior to treatment according to the methods described herein.

Note that what constitutes an improvement differs depending on the score being measured. For example, an improvement in an Eye Dryness score, the corneal score and the OSDI score is a decrease in the numerical value of the score. For the Schirmer's test, an improvement is typically an increase in the numerical value of the Schirmer's score.

In some of the embodiments described herein, the maintenance of the statistically significant improvement of the subject's score (e.g., EDS, Schirmer, corneal staining, or OSDI) means that the statistically significant improvement does not diminish by more than 10%, 20%, 30%, 40%, 50%, or 60%.

In some of the embodiments described herein, the statistically significant improvement in the subject's score (e.g., EDS, Schirmer, corneal, or OSDI) is maintained for at least 1 week, at least 1 month, at least 3 months, at least 6 months, at least 9 months, or at least 12 months. In some of the embodiments described herein, the statistically significant improvement in the subject's score is maintained for at least 30 days after treatment according to the methods described herein, wherein the statistically significant improvement does not diminish by more than 30%. In some embodiments, the statistically significant improvement in the subject's score is maintained for at least 1 month from administration of the first dose of the rAAV virion, and, optionally, the first dose of the treatment that increases tear production, wherein the statistically significant improvement does not diminish by more than 30%.

In some of the embodiments described herein, the statistically significant improvement in the subject's score (e.g., EDS, Schirmer, corneal, or OSDI) is maintained for at least 1 week, at least 1 month, at least 3 months, at least 6 months, at least 9 months, or at least 12 months after treatment according to the methods described herein, wherein the statistically significant improvement does not diminish by more than 10%, 20%, 30%, 40%, 50%, or 60% compared to the subject's score within 60 days after treatment according to the methods described herein.

In some of the embodiments described herein, the statistically significant improvement in the subject's score (e.g., EDS, Schirmer, corneal staining, or OSDI) is maintained for at least 1 week, at least 1 month, at least 3 months, at least 6 months, at least 9 months, or at least 12 months after treatment according to the methods described herein, wherein the statistically significant improvement does not diminish by more than 20% compared to the subject's corresponding Eye Dryness Score, corneal score, or OSDI score within 60 days after treatment according to the methods described herein.

### Dosing Timing And Method Of Administration

The schedule of doses administered to a subject depends on various considerations including the duration of effectiveness of each dose, the transduction efficiency of the rAAV virion, and the effect of the dose on the body. For example, wherein the patient's condition does not improve, upon the health provider's discretion, the method of treating an ocular condition as described herein, may be adjusted in dose or administered repeatedly in order to ameliorate or otherwise control or limit the symptoms of the subject's ocular condition. For instance, the period of time between administrations of one or more doses is extended, or the period of time between days the subject is administered one or more doses is extended. As a non-limiting example, administration of one or more doses is modified to administration of one or more doses after measuring symptoms of the ocular condition.

The term “dose”, as used herein, may refer to a dose of a pharmaceutical composition of the disclosure, or a dose of the treatment that reduces symptoms in an ocular condition.

In some of the embodiments described herein, a dose of the rAAV virion is a dose of a rAAV virion carrying an expression cassette. In such cases, delivery of an appropriate dose (e.g., effective amount) of the gene product is achieved by administering an appropriate amount/titer of the viral vector to the target site which allows expression of a therapeutically effective amount of the gene product over a period of time. In such cases, delivery of an appropriate dose (e.g., effective amount) of the gene product is achieved by administering an appropriate amount/titer of the viral vector to the target site which allows expression of an effective amount of the gene product over a period of time. In some embodiments, the viral vector is an rAAV virion. In some embodiments, the viral vector is administered to the lacrimal gland. In some embodiments, a dose of the viral vector results in the stable production of the gene product for a period of time (e.g., about 1 day, about 2 days, about 4 days, about 1 week, about 2 weeks, about 3 weeks, about 4 weeks, about 1 month, about 2 months, about 3 months, about 4 months, about 5 months, about 6 months, about 9 months, about 12 months, or longer). In some embodiments, a dose of the rAAV results in the stable production of the gene product for about 1 week. In some embodiments, a dose of the rAAV results in the stable production of the gene product for about 2 weeks. In some embodiments, a dose of the rAAV results in the stable production of the gene product for about 3 weeks. In some embodiments, a dose of the rAAV results in the stable production of the gene product for about 4 weeks. In some embodiments, a dose of the rAAV results in the stable production of the gene product for about 1 month. In some embodiments, a dose of the rAAV results in the stable production of the gene product for about 2 months. In some embodiments, a dose of the rAAV results in the stable production of the gene product for about 3 months. In some embodiments, a dose of the rAAV results in the stable production of the gene product for about 4 months. In some embodiments, a dose of the rAAV results in the stable production of the gene product for about 5 months. In some embodiments, a dose of the rAAV results in the stable production of the gene product for about 6 months. In some embodiments, a dose of the rAAV results in the stable production of the gene product for about 9 months. In some embodiments, a dose of the rAAV results in the stable production of the gene product for about 12 months.

In some embodiments, the rAAV is administered in repeat doses. In some embodiments, the rAAV is administered in 1, 2, 3, 4, 5, 6, 7, 8, 9, 10, or more doses. In some embodiments, the rAAV is administered in two doses. In some embodiments, the rAAV is administered in three doses. In some embodiments, the rAAV is administered in four doses. In some embodiments, the rAAV is administered in five doses. In some embodiments, the rAAV is administered in six doses. In some embodiments, the rAAV is administered in seven doses. In some embodiments, the rAAV is administered in eight doses. In some embodiments, the rAAV is administered in nine doses. In some embodiments, the rAAV is administered in ten doses.

In some embodiments, the method comprises delivering an effective amount of a gene product by administering a rAAV. In some embodiments, the method comprises delivering a first dose and one or more subsequent doses of the rAAV The one or more subsequent doses are administered after a period of time after the first dose. In some embodiments, this period of time between the first dose and the next subsequent dose is at least 1 day, at least 3 days, at least 1 week, at least 2 weeks, at least 3 weeks, at least 4 weeks, at least 1 month, at least 2 months, at least 4 months, at least 6 months, at least 9 months, at least 12 months, or longer. In some embodiments, this period of time between the first dose and the next subsequent dose is between 1-7 days, between 1-4 weeks, between 2-6 weeks, between 4-8 weeks, between 1-3 months, between 2-4 months, between 3-6 months, between 4-12 months, between 6-24 months. In some embodiments, the period of time between the one or more subsequent doses is at least 1 day, at least 3 days, at least 1 week, at least 2 weeks, at least 3 weeks, at least 4 weeks, at least 1 month, at least 2 months, at least 4 months, at least 6 months, at least 9 months, at least 12 months, or longer. In some embodiments, the period of time between the one or more subsequent doses is between 1-7 days, between 1-4 weeks, between 2-6 weeks, between 4-8 weeks, between 1-3 months, between 2-4 months, between 3-6 months, between 4-12 months, between 6-24 months.

In some embodiments, the doses are the same concentration of rAAV. In some embodiments, the doses are different concentrations of rAAV.

In some embodiments, the repeat doses are administered to the same eye or lacrimal gland. In some embodiments, the repeat doses are administered to at least one eye or lacrimal gland.

### Pharmaceutical Compositions and Kits

In some embodiments, the disclosure provides a pharmaceutical composition comprising an rAAV virion described herein. In some embodiments, the pharmaceutical composition comprises an rAAV virion described herein, and a pharmaceutically acceptable carrier, delivery agent, or excipient.

In some embodiments, the disclosure provides use of an rAAV virion or pharmaceutical composition described herein, in the manufacture of a medicament for treatment of an ocular disease, disorder, or condition. In some embodiments, the disclosure provides use of an rAAV virion or pharmaceutical composition herein for use, or adaptable for use, in the treatment of an ocular disease, disorder, or condition.

In some embodiments, the pharmaceutically acceptable carrier comprises phosphate buffered saline. In some embodiments, the pharmaceutical composition is formulated to be compatible with its intended route of administration (e.g., intralacrimal). In some embodiments, the pharmaceutical composition is formulated for administration into the lacrimal gland. In some embodiments, the pharmaceutical composition is formulated for administration onto the ocular surface.

Any concentration of viral particles suitable to effectively transduce cells of the eye and/or lacrimal gland can be prepared for contacting cells of the eye and/or lacrimal gland in vitro or in vivo. For example, the viral particles may be formulated at a concentration of 10⁸ vector genomes per ml or more, for example, 5×10⁸ vector genomes per mL; 10⁹ vector genomes per mL; 5×10⁹ vector genomes per mL, 10¹⁰ vector genomes per mL, 5×10¹⁰ vector genomes per mL; 10¹¹ vector genomes per mL; 5×10¹¹ vector genomes per mL; 10¹² vector genomes per mL; 5×10¹² vector genomes per mL; 10¹³ vector genomes per mL; 1.5×10¹³ vector genomes per mL; 3×10¹³ vector genomes per mL; 5×10¹³ vector genomes per mL; 7.5×10¹³ vector genomes per mL; 9×10¹³ vector genomes per mL; 1×10¹⁴ vector genomes per mL, 5×10¹⁴ vector genomes per mL or more, but typically not more than 1×10¹⁵ vector genomes per mL. Similarly, any total number of viral particles suitable to provide appropriate transduction of cells of the eye and/or lacrimal gland to confer the desired effect or treat the disease can be administered to the mammal or to the primate's eye. In various embodiments, at least 10⁷; 5×10⁷; 10⁸; 5×10⁸; 10⁹; 5×10⁹, 10¹⁰, 5×10¹⁰; 10¹¹; 5×10¹¹; 10¹²; 5×10¹²; 10¹³; 1.5×10¹³; 3×10¹³; 5×10¹³; 7.5×10¹³; 9×10¹³, 1×10¹⁴ viral particles, or 5×10¹⁴ viral particles or more, but typically not more than 1×10¹⁵ viral particles are injected per eye. In various embodiments, at least 10⁸; 5×10⁸; 10⁹; 5×10⁹, 10¹⁰, 5×10¹⁰; 10¹¹; 5×10¹¹; 10¹², 5×10¹²; 10¹³; 1.5×10¹³; 3×10¹³; 5×10¹³; 7.5×10¹³, 9×10¹³, 1×10¹⁴ viral particles, or 5×10¹⁴ viral particles or more, but typically not more than 1×10¹⁵ viral particles are injected per eye. Any suitable number of administrations of the vector to the mammal or the primate eye can be made. In one embodiment, the methods comprise a single administration; in other embodiments, multiple administrations are made over time as deemed appropriate by an attending clinician.

The subject viral vector may be formulated into any suitable unit dosage, including, without limitation, 1×10⁸ vector genomes or more, for example, 1×10⁹, 1×10¹⁰, 1×10¹¹, 1×10¹², or 1×10¹³ vector genomes or more, in certain instances, 1×10¹⁴ vector genomes, but usually no more than 4×10¹⁵ vector genomes. In some embodiments, the viral vector is formulated into any suitable unit dosage, including, without limitation, 1×10⁷, 1×10⁸, 1×10⁹, 1×10¹⁰, 1×10¹¹, 1×10¹², or 1×10¹³ vector genomes or more. In some cases, the unit dosage is at most about 5×10¹⁵ vector genomes, e.g., 1×10¹⁴ vector genomes or less, for example 1×10¹³, 1×10¹², 1×10¹¹, 1×10¹⁰, or 1×10⁹ vector genomes or less, in certain instances 1×10⁸ vector genomes or less, and typically no less than 1×10⁸ vector genomes. In some cases, the unit dosage is at most about 5×10¹⁵ vector genomes, e.g., 1×10¹⁴ vector genomes or less, for example 1×10¹³, 1×10¹², 1×10¹¹, 1×10¹⁰, 1×10⁹, 1×10⁸, or 1×10⁷ vector genomes or less. In some cases, the unit dosage is 1×10¹⁰ to 1×10¹¹ vector genomes. In some cases, the unit dosage is 1×10¹⁰ to 3×10¹² vector genomes. In some cases, the unit dosage is 1×10⁹ to 3×10¹³ vector genomes. In some cases, the unit dosage is 1×10⁸ to 3×10¹⁴ vector genomes.

In some cases, the unit dosage of pharmaceutical composition may be measured using multiplicity of infection (MOI). By MOI it is meant the ratio, or multiple, of vector or viral genomes to the cells to which the nucleic acid may be delivered. In some cases, the MOI may be 1×10⁶. In some cases, the MOI may be 1×10⁵-1×10⁷. In some cases, the MOI may be 1×10⁴-1×10⁸. In some cases, recombinant viruses of the disclosure are at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ MOI. In some cases, recombinant viruses of this disclosure are 1×10⁸ to 3×10¹⁴ MOI. In some cases, recombinant viruses of the disclosure are at most about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ MOI.

In some embodiments, the amount of pharmaceutical composition comprises about 1×10⁸ to about 1×10¹⁵ recombinant viruses, about 1×10⁹ to about 1×10¹⁴ recombinant viruses, about 1×10¹⁰ to about 1×10¹³ recombinant viruses, or about 1×10¹¹ to about 3×10¹² recombinant viruses.

In preparing the subject rAAV compositions, any host cells for producing rAAV virions may be employed, including, for example, mammalian cells (e.g., 293 cells), insect cells (e.g., SF9 cells), microorganisms and yeast. Host cells can also be packaging cells in which the AAV rep and cap genes are stably maintained in the host cell or producer cells in which the rAAV virion genome is stably maintained and packaged. Exemplary packaging and producer cells are derived from SF-9, 293, A549 or HeLa cells. rAAV virions are purified and formulated using standard techniques known in the art.

In some embodiments, the disclosure provides for use of an rAAV virion described herein in the manufacture of a medicament. In some embodiments, the disclosure provides for use of an rAAV virion described herein in the manufacture of a medicament for use in a method described herein.

In some embodiments, the disclosure provides a kit comprising an rAAV herein, and instructions for use. In some embodiments, the kit comprises an rAAV herein, and a package insert containing instructions for use of the kit. In some embodiments, the kit comprises an rAAV herein, and a pharmaceutically acceptable carrier, or a pharmaceutical composition comprising the rAAV and instructions for treating or delaying progression of a disease, disorder, or condition described herein in a subject in need thereof.

### EXEMPLARY EMBODIMENTS

The disclosure relates to the following embodiments. Throughout this section, the term embodiment is abbreviated as “E” followed by an ordinal. For example, EI-1 is equivalent to Embodiment I-1.

Embodiment I-1. A method of treating an ocular condition in a subject in need thereof, the method comprising administering a recombinant adeno-associated virus (rAAV) virion, the rAAV virion comprising an AAV capsid and an expression cassette comprising a polynucleotide encoding a neurotrophic factor operatively linked to a promoter, to at least one eye of the subject or to at least one lacrimal gland of the eye of the subject.

Embodiment I-2. The method of embodiment I-1, wherein the rAAV virion is administered to a lacrimal gland of the subject.

Embodiment I-3. The method of embodiment I-2, wherein the lacrimal gland is the main lacrimal gland or any one of the Wolfring's glands or the Krause's glands of the subject.

Embodiment I-4. The method of embodiment I-2, wherein the lacrimal gland is the main lacrimal gland.

Embodiment I-5. The method of any one of embodiments I-2 to I-4, wherein cells within the lacrimal gland are transduced by the rAAV virion.

Embodiment I-6. The method of embodiment I-5, wherein the transduced cells within the lacrimal gland express an effective amount of the neurotrophic factor into the tear film and optionally onto the ocular surface of the subject.

Embodiment I-7. The method of any one of embodiments I-1 to I-6, wherein the neurotrophic factor is a Nerve Growth Factor (NGF) protein.

Embodiment I-8. The method of embodiment I-7, wherein the polynucleotide encoding the NGF protein comprises a sequence that shares at least 95% identity to SEQ ID NO: 2.

Embodiment I-9. The method of embodiment I-7, wherein the NGF protein comprises a sequence that shares at least 95% identity to SEQ ID NO: 1.

Embodiment I-10. The method of embodiment I-7, wherein the NGF protein comprises SEQ ID NO: 1.

Embodiment I-11. The method of embodiment I-7, wherein the polynucleotide encoding the NGF protein comprises SEQ ID NO: 2.

Embodiment I-12. The method of any one of embodiments I-1 to I-6, wherein the neurotrophic factor is a Glial Derived Neurotrophic Factor (GDNF) protein.

Embodiment I-13. The method of embodiment I-12, wherein the polynucleotide encoding the GDNF protein comprises a sequence that shares at least 95% identity to SEQ ID NO: 4.

Embodiment I-14. The method of embodiment I-12, wherein the GDNF protein comprises a sequence that shares at least 95% identity to SEQ ID NO: 3.

Embodiment I-15. The method of embodiment I-12, wherein the GDNF protein comprises SEQ ID NO: 3.

Embodiment I-16. The method of embodiment I-12, wherein the polynucleotide encoding the GDNF protein comprises SEQ ID NO: 4.

Embodiment I-17. The method of any one of embodiments I-1 to I-16, wherein the AAV capsid shares at least 95%, 98%, or 100% identity to AAV2 VP1 (SEQ ID NO: 6), AAV2 VP3 (SEQ ID NO: 8), AAV5 (SEQ ID NO: 10), AAV8 (SEQ ID NO: 12), or AAV9 (SEQ ID NO: 14).

Embodiment I-18. The method of any one of embodiments I-1 to I-17, wherein the promoter is a CAG promoter (SEQ ID NO: 5).

Embodiment I-19. The method of any one of embodiments I-1 to I-18, wherein the ocular condition is a chemical burn of the ocular surface, corneal wound, corneal ulcer, persistent epithelial defect, dry eye disease, neurotrophic keratitis, herpes simplex viral infection of the trigeminal nerve and/or the eye, varicella zoster virus infection of the trigeminal nerve and/or the eye, and diabetic complications of the corneal nerves.

Embodiment I-20. The method of any one of embodiments I-1 to I-19, wherein one or more symptoms of the ocular condition are reduced compared to the symptoms of the ocular condition before administration of the rAAV virion.

Embodiment I-21. The method of any one of embodiments I-1 to I-20, wherein one or more symptoms of the ocular condition are reduced compared to the symptoms of the ocular condition in an untreated control subject.

Embodiment I-22. The method of any one of embodiments I-1 to I-21, wherein one or more symptoms of the ocular condition are reduced compared to the symptoms of the ocular condition in a contralateral eye.

Embodiment I-23. A recombinant adeno-associated virus (rAAV) virion, comprising an AAV capsid and an expression cassette, wherein the expression cassette comprises a polynucleotide encoding a neurotrophic factor operatively linked to a promoter.

Embodiment I-24. The rAAV virion of embodiment I-23, wherein the neurotrophic factor is a Nerve Growth Factor (NGF) protein.

Embodiment I-25. The rAAV virion of embodiment I-24, wherein the polynucleotide encoding the NGF protein comprises a sequence that shares at least 95% identity to SEQ ID NO: 2 or SEQ ID NO: 17.

Embodiment I-26. The rAAV virion of embodiment I-24, wherein the NGF protein comprises a sequence that shares at least 95% identity to SEQ ID NO: 1.

Embodiment I-27. The rAAV virion of embodiment I-24, wherein the NGF protein comprises SEQ ID NO: 1.

Embodiment I-28. The rAAV virion of embodiment I-24, wherein the polynucleotide encoding the NGF protein comprises SEQ ID NO: 2 or SEQ ID NO: 17.

Embodiment I-29. The rAAV virion of embodiment I-23, wherein the neurotrophic factor is a Glial Derived Neurotrophic Factor (GDNF) protein.

Embodiment I-30. The rAAV virion of embodiment I-29, wherein the polynucleotide encoding the GDNF protein comprises a sequence that shares at least 95% identity to SEQ ID NO: 4 or SEQ ID NO: 18.

Embodiment I-31. The rAAV virion of embodiment I-29, wherein the GDNF protein comprises a sequence that shares at least 95% identity to SEQ ID NO: 3.

Embodiment I-32. The rAAV virion of embodiment I-29, wherein the GDNF protein comprises SEQ ID NO: 3.

Embodiment I-33. The rAAV virion of embodiment I-29, wherein the polynucleotide encoding the GDNF protein comprises SEQ ID NO: 4 or SEQ ID NO: 18.

Embodiment I-34. The rAAV virion of any one of embodiments I-23 to I-33, wherein the AAV capsid comprises a VP3 that shares at least 95%, 98%, or 100% identity to AAV2 VP1 (SEQ ID NO: 6), AAV2 VP3 (SEQ ID NO: 8), AAV5 (SEQ ID NO: 10), AAV8 (SEQ ID NO: 12), or AAV9 (SEQ ID NO: 14).

Embodiment I-35. The rAAV virion of any one of embodiments I-23 to I-34, wherein the promoter is a CAG promoter (SEQ ID NO: 5) or a CMV promoter (SEQ ID NO: 16).

Embodiment I-36. The rAAV virion of embodiment I-23, wherein the expression cassette comprises a sequence that shares at least 90%, 95%, 96%, 97%, 98%, 99%, or 100% identity to SEQ ID NO: 23.

Embodiment I-37. The rAAV virion of embodiment I-23, wherein the expression cassette comprises a sequence that shares at least 90%, 95%, 96%, 97%, 98%, 99%, or 100% identity to SEQ ID NO: 24.

Embodiment I-38. The rAAV virion of embodiment I-23, wherein the expression cassette comprises a sequence that shares at least 90%, 95%, 96%, 97%, 98%, 99%, or 100% identity to SEQ ID NO: 25.

Embodiment I-39. A recombinant adeno-associated virus (rAAV) virion comprising an expression cassette comprising a polynucleotide of SEQ ID NO: 25.

Embodiment I-40. The rAAV of embodiment I-39, wherein the rAAV comprises an AAV capsid.

Embodiment I-41. The rAAV of embodiment I-40, wherein the AAV capsid shares at least 95%, 98%, or 100% identity to AAV2 VP1 (SEQ ID NO: 6), AAV2 VP3 (SEQ ID NO: 8), AAV5 (SEQ ID NO: 10), AAV8 (SEQ ID NO: 12), or AAV9 (SEQ ID NO: 14).

Embodiment I-42. A composition comprising an rAAV virion, wherein the rAAV virion comprises:
(a) an AAV capsid, and
(b) an expression cassette, wherein the expression cassette comprises a polynucleotide sharing at least 95% identity to SEQ ID NO: 2 or SEQ ID NO: 17, and wherein the polynucleotide is linked to a promoter.

Embodiment I-43. A composition comprising an rAAV virion, wherein the rAAV virion comprises:
(a) an AAV capsid, and
(b) an expression cassette, wherein the expression cassette comprises a polynucleotide sharing at least 95% identity to SEQ ID NO: 4 or SEQ ID NO: 18, and wherein the polynucleotide is linked to a promoter.

Embodiment I-44. A composition comprising an rAAV virion, wherein the rAAV virion comprises:
(a) an AAV2, AAV5, AAV8, or AAV9 capsid, and
(b) an expression cassette, wherein the expression cassette comprises a polynucleotide sharing at least 95% identity to SEQ ID NO: 2 or SEQ ID NO: 17, and wherein the polynucleotide is linked to a promoter.

Embodiment I-45. A composition comprising an rAAV virion, wherein the rAAV virion comprises:
(a) an AAV2, AAV5, AAV8, or AAV9 capsid, and
(b) an expression cassette, wherein the expression cassette comprises a polynucleotide sharing at least 95% identity to SEQ ID NO: 4 or SEQ ID NO: 18, and wherein the polynucleotide is linked to a promoter.

Embodiment I-46. The composition of any one of embodiments I-42 to I-45, wherein the AAV capsid shares at least 95%, 98%, or 100% identity to AAV2 VP1 (SEQ ID NO: 6), AAV2 VP3 (SEQ ID NO: 8), AAV5 (SEQ ID NO: 10), AAV8 (SEQ ID NO: 12), or AAV9 (SEQ ID NO: 14).

Embodiment I-47. The composition of any one of embodiments I-42 to I-45, wherein the AAV capsid is AAV2.

Embodiment I-48. The composition of any one of embodiments I-42 to I-45, wherein the AAV capsid is AAV5.

Embodiment I-49. The composition of any one of embodiments I-42 to I-45, wherein the AAV capsid is AAV9.

Embodiment I-50. A composition comprising an rAAV virion, wherein the rAAV virion comprises:
(a) an AAV2 capsid, and
(b) an expression cassette, wherein the expression cassette comprises a polynucleotide sharing at least 95% identity to SEQ ID NO: 25.

Embodiment I-51. A composition comprising an rAAV virion, wherein the rAAV virion comprises:
(a) an AAV5 capsid, and
(b) an expression cassette, wherein the expression cassette comprises a polynucleotide sharing at least 95% identity to SEQ ID NO: 25.

Embodiment I-52. A composition comprising an rAAV virion, wherein the rAAV virion comprises:
(a) an AAV9 capsid, and
(b) an expression cassette, wherein the expression cassette comprises a polynucleotide sharing at least 95% identity to SEQ ID NO: 25.

Embodiment I-53. A pharmaceutical composition comprising the rAAV virion of any one of embodiments I-23 to I-41, or the composition of any one of embodiments I-42 to I-52, and a pharmaceutically acceptable carrier.

Embodiment I-54. The pharmaceutical composition of embodiment I-53, wherein the composition comprises about 1×10⁷ to about 1×10¹⁴ genome copies per milliliter of the rAAV virion.

Embodiment I-55. The pharmaceutical composition of embodiment I-53, wherein the composition comprises about 1×10¹² to about 6.2×10¹² genome copies per milliliter of the rAAV virion.

Embodiment I-56. The pharmaceutical composition of any one of embodiments I-53 to I-55, wherein the composition is formulated for administration into the lacrimal gland.

Embodiment I-57. The pharmaceutical composition of any one of embodiments I-53 to I-55, wherein the composition is formulated for administration onto the ocular surface.

Embodiment I-58. The pharmaceutical composition of any one of embodiments I-53 to I-57, wherein the composition is formulated for use, or adaptable for use, in the treatment of an ocular disease, disorder, or condition.

Embodiment I-59. A method of treating an ocular condition in a subject in need thereof, the method comprising administering an effective amount of the pharmaceutical composition of any one of embodiments I-53 to I-58 to the eye of the subject to an ocular secretory gland of the subject.

Embodiment I-60. The method of embodiment I-59, wherein the rAAV virion is administered to the lacrimal gland of the subject.

Embodiment I-61. The method of embodiment I-60, wherein cells within the lacrimal gland are transduced by the rAAV virion.

Embodiment I-62. The method of embodiment I-61, wherein the transduced cells within the lacrimal gland express an effective amount of the neurotrophic factor into the tear film and optionally onto the ocular surface of the subject.

Embodiment I-63. The method of any one of embodiments I-59 to I-62, wherein the ocular condition is neurotrophic keratitis.

Embodiment I-64. The method of any one of embodiments I-59 to I-63, wherein one or more symptoms of the ocular condition are reduced compared to the symptoms of the ocular condition before administration of the rAAV virion.

Embodiment I-65. The method of any one of embodiments I-59 to I-64, wherein one or more symptoms of the ocular condition are reduced compared to the symptoms of the ocular condition in an untreated control subject.

Embodiment I-66. The method of any one of embodiments I-59 to I-65, wherein one or more symptoms of the ocular condition are reduced compared to the symptoms of the ocular condition in a contralateral eye.

Embodiment I-67. The rAAV virion of any one of embodiments I-23 to I-41, the composition of any one of embodiments I-42 to I-52, or the pharmaceutical composition of any one of embodiments I-53 to I-58 for use in the method of any one of embodiments I-1 to I-22.

Embodiment I-68. Use of the rAAV virion of any one of embodiments I-23 to I-41 or the composition of any one of embodiments I-42 to I-52 in the manufacture of a medicament for use in the method of any one of embodiments I-1 to I-22.

Embodiment I-69. A kit comprising the rAAV virion of any one of embodiments I-23 to I-41, the composition of any one of embodiments I-42 to I-52, or the pharmaceutical composition of any one of embodiments I-53 to I-58, and instructions for use in the method of any one of embodiments I-1 to I-22.

Embodiment I-70. A recombinant adeno-associated virus (rAAV) virion for use in a method of treating an ocular condition in a subject in need thereof, the method comprising administering a recombinant adeno-associated virus (rAAV) virion, the rAAV virion comprising an AAV capsid and an expression cassette comprising a polynucleotide encoding a neurotrophic factor operatively linked to a promoter, to at least one eye of the subject or to at least one lacrimal gland of the eye of the subject.

Embodiment I-71. The rAAV virion for use of embodiment I-70, wherein the rAAV virion is administered to a lacrimal gland of the subject.

Embodiment I-72. The rAAV virion for use of embodiment I-71, wherein cells within the lacrimal gland are transduced by the rAAV virion.

Embodiment I-73. The rAAV virion for use of any one of embodiments I-70 to I-72, wherein the neurotrophic factor is a Nerve Growth Factor (NGF) protein.

Embodiment I-74. The rAAV virion for use of any one of embodiments I-70 to I-72, wherein the neurotrophic factor is a Glial Derived Neurotrophic Factor (GDNF) protein.

Embodiment I-75. The rAAV virion for use of any one of embodiments I-70 to I-74, wherein the ocular condition is selected from chemical burn of the ocular surface, corneal wound, persistent epithelial defect, dry eye disease, neurotrophic keratitis, herpes simplex viral infection of the trigeminal nerve and/or the eye, varicella zoster virus infection of the trigeminal nerve and/or the eye, and diabetic complications of the corneal nerves.

Some embodiments of this disclosure relate to Embodiment II, as follows:

Embodiment II-1. A method of treating an ocular condition in a subject in need thereof, the method comprising administering a recombinant adeno-associated virus (rAAV) virion, the rAAV virion comprising an AAV capsid and an expression cassette comprising a polynucleotide encoding a neurotrophic factor operatively linked to a promoter, to at least one eye of the subject or to at least one lacrimal gland of the eye of the subject.

Embodiment II-2. The method of embodiment II-1, wherein the rAAV virion is administered to a lacrimal gland of the subject.

Embodiment II-3. The method of embodiment II-2, wherein the lacrimal gland is the main lacrimal gland.

Embodiment II-4. The method of embodiment II-2, wherein the lacrimal gland is the main lacrimal gland or any one of the Wolfring's glands or the Krause's glands of the subject.

Embodiment II-5. The method of any one of embodiments II-2 to II-4, wherein cells within the lacrimal gland is transduced by the rAAV virion.

Embodiment II-6. The method of embodiment II-5, wherein the transduced cells within the lacrimal gland express a therapeutically effective amount of the neurotrophic factor into the tear film and optionally onto the ocular surface of the subject.

Embodiment II-7. The method of any one of embodiments II-1 to II-6, wherein the neurotrophic factor is a Nerve Growth Factor (NGF) protein.

Embodiment II-8. The method of embodiment II-7, wherein the polynucleotide encoding the NGF protein comprises a sequence that shares at least 95% identity to SEQ ID NO: 2.

Embodiment II-9. The method of embodiment II-7, wherein the NGF protein comprises a sequence that shares at least 95% identity to SEQ ID NO: 1.

Embodiment II-10. The method of embodiment II-7, wherein the NGF protein comprises SEQ ID NO: 1.

Embodiment II-11. The method of embodiment II-7, wherein the polynucleotide encoding the NGF protein comprises SEQ ID NO: 2.

Embodiment II-12. The method of any one of embodiments II-1 to II-6, wherein the neurotrophic factor is a Glial Derived Neurotrophic Factor (GDNF) protein.

Embodiment II-13. The method of embodiment II-12, wherein the polynucleotide encoding the GDNF protein comprises a sequence that shares at least 95% identity to SEQ ID NO: 4.

Embodiment II-14. The method of embodiment II-12, wherein the GDNF protein comprises a sequence that shares at least 95% identity to SEQ ID NO: 3.

Embodiment II-15. The method of embodiment II-12, wherein the GDNF protein comprises SEQ ID NO: 3.

Embodiment II-16. The method of embodiment II-12, wherein the polynucleotide encoding the GDNF protein comprises SEQ ID NO: 4.

Embodiment II-17. The method of any one of embodiments II-1 to II-6, wherein the AAV capsid shares at least 95%, 98%, or 100% identity to AAV2 VP1 (SEQ ID NO: 6), AAV2 VP3 (SEQ ID NO: 8), AAV5 (SEQ ID NO: 10), AAV8 (SEQ ID NO: 12), or AAV9 (SEQ ID NO: 14).

Embodiment II-18. The method of any one of embodiments II-1 to II-16, wherein the promoter is a CAG promoter (SEQ ID NO: 5).

Embodiment II-19. The method of any one of embodiments II-1 to II-18, wherein the ocular condition is chemical burn of the ocular surface, corneal wound, persistent epithelial defect, dry eye disease, neurotrophic keratitis, herpes simplex viral infection of the trigeminal nerve and/or the eye, varicella zoster virus infection of the trigeminal nerve, and/or the eye or diabetic complications of the corneal nerves.

Embodiment II-20. The method of any one of embodiments II-1 to II-19, wherein one or more symptoms of the ocular condition are reduced compared to the symptoms of the ocular condition before administration of the rAAV virion.

Embodiment II-21. The method of any one of embodiments II-1 to II-20, wherein one or more symptoms of the ocular condition are reduced compared to the symptoms of the ocular condition in an untreated control subject.

Embodiment II-22. The method of any one of embodiments II-1 to II-21, wherein one or more symptoms of the ocular condition are reduced compared to the symptoms of the ocular condition in a contralateral eye.

Embodiment II-23. A recombinant adeno-associated virus (rAAV) virion, comprising an AAV capsid and an expression cassette comprising a polynucleotide encoding a neurotrophic factor operatively linked to a promoter.

Embodiment II-24. The rAAV virion of embodiment II-23, wherein the neurotrophic factor is a Nerve Growth Factor (NGF) protein.

Embodiment II-25. The rAAV virion of embodiment II-24, wherein the polynucleotide encoding the NGF protein comprises a sequence that shares at least 95% identity to SEQ ID NO: 2 or SEQ ID NO: 17.

Embodiment II-26. The rAAV virion of embodiment II-24, wherein the NGF protein comprises a sequence that shares at least 95% identity to SEQ ID NO: 1.

Embodiment II-27. The rAAV virion of embodiment II-24, wherein the NGF protein comprises SEQ ID NO: 1.

Embodiment II-28. The rAAV virion of embodiment II-24, wherein the polynucleotide encoding the NGF protein comprises SEQ ID NO: 2 or SEQ ID NO: 17.

Embodiment II-29. The rAAV virion of embodiment II-23, wherein the neurotrophic factor is a Glial Derived Neurotrophic Factor (GDNF) protein.

Embodiment II-30. The rAAV virion of embodiment II-29, wherein the polynucleotide encoding the GDNF protein comprises a sequence that shares at least 95% identity to SEQ ID NO: 4 or SEQ ID NO: 18.

Embodiment II-31. The rAAV virion of embodiment II-29, wherein the GDNF protein comprises a sequence that shares at least 95% identity to SEQ ID NO: 3.

Embodiment II-32. The rAAV virion of embodiment II-29, wherein the GDNF protein comprises SEQ ID NO: 3.

Embodiment II-33. The rAAV virion of embodiment II-29, wherein the polynucleotide encoding the GDNF protein comprises SEQ ID NO: 4 or SEQ ID NO: 19.

Embodiment II-34. The rAAV virion of any one of embodiments II-23 to II-33, wherein the AAV capsid comprises a VP3 that shares at least 95%, 98%, or 100% identity to AAV2 VP1 (SEQ ID NO: 6), AAV2 VP3 (SEQ ID NO: 8), AAV5 (SEQ ID NO: 10), AAV8 (SEQ ID NO: 12), or AAV9 (SEQ ID NO: 14).

Embodiment II-35. The rAAV virion of any one of embodiments II-23 to II-34, wherein the promoter is a CAG promoter (SEQ ID NO: 5) or a CMV promoter (SEQ ID NO: 16).

Embodiment II-36. The rAAV virion of embodiment II-23, wherein the expression cassette comprises a sequence that shares at least 90%, 95%, 96%, 97%, 98%, 99%, or 100% identity to SEQ ID NO: 23.

Embodiment II-37. The rAAV virion of embodiment II-23, wherein the expression cassette comprises a sequence that shares at least 90%, 95%, 96%, 97%, 98%, 99%, or 100% identity to SEQ ID NO: 24.

Embodiment II-38. A pharmaceutical composition comprising the rAAV virion of any one of embodiments II-23 to II-37 and a pharmaceutically acceptable carrier.

Embodiment II-39. The pharmaceutical composition of embodiment II-38, wherein the composition comprises about 1×10⁹ to about 1×10¹⁴ genome copies per milliliter of the rAAV virion.

Embodiment II-40. The pharmaceutical composition of embodiment II-38, wherein the composition comprises about 1×10¹² to about 6.2×10¹² genome copies per milliliter of the rAAV virion.

Embodiment II-41. A method of treating an ocular condition in a subject in need thereof, the method comprising administering a therapeutically effective amount of the pharmaceutical composition of any one of embodiments II-38 to II-40 to the eye of the subject to an ocular secretory gland of the subject.

Embodiment II-42. The method of embodiment II-41, wherein the rAAV virion is administered to the lacrimal gland of the subject.

Embodiment II-43. The method of embodiment II-42, wherein cells within the lacrimal gland are transduced by the rAAV virion.

Embodiment II-44. The method of embodiment II-43, wherein the transduced cells within the lacrimal gland express a therapeutically effective amount of the neurotrophic factor into the tear film and optionally onto the ocular surface of the subject.

Embodiment II-45. The method of any one of embodiments II-41 to II-44, wherein the ocular condition is neurotrophic keratitis.

Embodiment II-46. The method of any one of embodiments II-41 to II-45, wherein one or more symptoms of the ocular condition are reduced compared to the symptoms of the ocular condition before administration of the rAAV virion.

Embodiment II-47. The method of any one of embodiments II-41 to II-46, wherein one or more symptoms of the ocular condition are reduced compared to the symptoms of the ocular condition in an untreated control subject.

Embodiment II-48. The method of any one of embodiments II-41 to II-46, wherein one or more symptoms of the ocular condition are reduced compared to the symptoms of the ocular condition in a contralateral eye.

Embodiment II-49. The rAAV virion of any one of embodiments II-23 to II-34 or the pharmaceutical composition of any one of embodiments II-35 to II-38 for use in the method of any one of embodiments II-1 to II-22.

Embodiment II-50. Use of the rAAV virion of any one of embodiments II-23 to II-37 in the manufacture of a medicament for use in the method of any one of embodiments II-1 to II-22.

Embodiment II-51. A kit comprising the rAAV virion of any one of embodiments II-23 to II-37 or the pharmaceutical composition of any one of embodiments II-38 to II-40, and instructions for use in the method of any one of embodiments II-1 to II-22.

### EXAMPLES

The following specific examples are to be construed as merely illustrative, and not limitative of the remainder of the disclosure in any way whatsoever.

### Example 1

Expression of GFP Transgene in Lacrimal Gland Delivered by rAAV via Intralacrimal Injection

This example shows a 9-day pilot study of single dose rAAV virions administered as an Intralacrimal Gland Injection to Dutch-Belted Rabbits. It evaluates the effectiveness and tolerability of a panel of rAAV virion embodiments administered one time via injection to the lacrimal gland. Each rAAV virion composition in the panel was tested at two concentrations (1×10¹² GC/mL and 6.2×10¹² GC/mL). The panel of rAAV virions include embodiments with capsid proteins having AAV2, AAV5, AAV8, and AAV9 serotypes. The expression cassette delivered by the rAAV virion encodes an enhanced green fluorescent protein (eGFP) transgene that is operatively linked to a CMV promoter (as depicted in FIG. 1C). This approach evaluated the feasibility of capsid protein serotypes AAV2, AAV5, AAV8, and AAV9 to deliver a transgene to cells in the lacrimal gland resulting in measurable CAG promoter-driven expression of the transgene within the cells.

Animal studies were carried out in the Charles River Laboratories (CRL) facilities by CRL staff scientific personnel.

### Animal Test System, Husbandry, and In-life Monitoring

The animals used in this study were male Dutch-Belted rabbits between the ages of 4 to 5 months and weighed between 1.3 to 2.3 kg. Animals were acclimated for 10 days prior to the start of treatment. Each animal was housed individually and cared for using standard caregiving protocols including regular environmental conditions, feeding schedules, and veterinary care.

rAAV Virion Compositions and Formulations

In this study, the panel of compositions comprising rAAV virions containing an expression cassette encoding an eGFP transgene operatively linked to a CAG promoter are provided for intralacrimal injection under the conditions in Table 4. Each composition contains an rAAV virion with a different AAV capsid protein serotype. The compositions are labeled as OC-100a-d, each corresponding to a different AAV capsid protein serotype. Dose formulations for intralacrimal injection were prepared using clean procedures at the target concentrations described below in Table 4 by diluting with phosphate buffered saline solution.

TABLE 4
Summary of rAAV Compositions

| | rAAV | rAAV | rAAV | rAAV |
|---|---|---|---|---|
| | Composition | Composition | Composition | Composition |
| | 1 | 2 | 3 | 4 |
| | (OC-100a) | (OC-100b) | (OC-100c) | (OC-100d) |
| AAV Capsid | AAV2 | AAV5 | AAV8 | AAV9 |
| Serotype | | | | |
| Physical | Clear | Clear | Clear | Clear |
| Description | Liquid | Liquid | Liquid | Liquid |
| Concentration | 1.5 × 10¹³ | 1.3 × 10¹³ | 1.1 × 10¹³ | 4 × 10¹³ |
| | GC/mL | GC/mL | GC/mL | GC/mL |
| Storage | −80° C. | −80° C. | −80° C. | −80° C. |
| Conditions | | | | |

Intralacrimal Injection of rAAV Compositions

Animals were dosed via intralacrimal injection on day 1 of the study. A summary of the formulation concentration for each composition tested, dose volume, dose frequency, and number of animals and lacrimal glands is found in Table 5. Prior to the injection, animals were anesthetized by intramuscular injection of dexmedetomidine (0.25 mg/kg) followed by an isoflurane/oxygen mix through a mask to maintain anesthesia, if necessary. A topical antibiotic was applied to each eye after dose administration.

TABLE 5
Summary of Experimental Design

| | | Formulation | | | | Number of |
|---|---|---|---|---|---|---|
| | | Concentration | Dose | | | Lacrimal |
| Group | rAAV | (genome copies | Volume | Dose | Number of | Glands |
| No. | Composition | (GC)/mL) | (μL/gland/dose) | Frequency | Animals | Injected |
| 1 | OC-100a | 1 × 10¹² | 100 | once | 1 | 2 |
| 2 | OC-100a | 6.2 × 10¹² | 100 | once | 2 | 4 |
| 3 | OC-100b | 1 × 10¹² | 100 | once | 1 | 2 |
| 4 | OC-100b | 6.2 × 10¹² | 100 | once | 2 | 4 |
| 5 | OC-100c | 1 × 10¹² | 100 | once | 1 | 2 |
| 6 | OC-100c | 6.2 × 10¹² | 100 | once | 2 | 4 |
| 7 | OC-100d | 1 × 10¹² | 100 | once | 1 | 2 |
| 8 | OC-100d | 6.2 × 10¹² | 100 | once | 2 | 4 |

### Bioanalysis

Blood was collected on Day 1 before dosing, and again on Day 8 and 9 from an auricular vessel from all animals. Blood samples were placed on ice until plasma was separated by centrifugation. Plasma samples were separated into 250 μL aliquots and frozen at -80° ° C.for subsequent analysis.

A Schirmer tear test was performed to collect eye moisture from the animals on Day 8 and 9. Test strips were placed inside the lower eyelid for approximately 1 minute. The paper was removed and placed into separate tubes and frozen at −80° C. for subsequent analysis.

Plasma samples and Schirmer's test strips were analyzed for concentration of eGFP and eGFP mRNA concentration using a validated procedure at Syneos analytical laboratories. Anaylsis in the plasma samples detected no eGFP protein or eGFP mRNA, indicating that intralacrimal delivery of the rAAV did not induce cell lysis. This result suggests the intralacrimal route of administration may be safely used for delivering rAAV.

### Immunohistochemistry of Lacrimal Gland Tissue

Animals were euthanized on day 9 following collection of blood and eye moisture by intravenous injection of sodium pentobarbital. Five sagittal sections of the left eye and sections of the left and right lacrimal glands were prepared for immunohistochemistry (IHC) according to Charles River lab standard operating procedure. Lacrimal gland IHC samples were stained for eGFP and subject to microscopic evaluation.

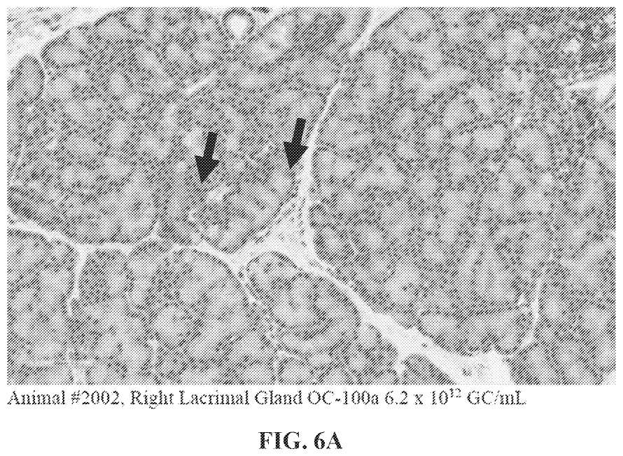

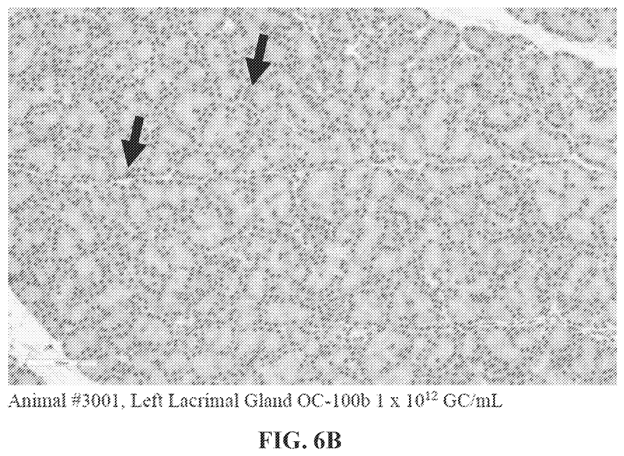

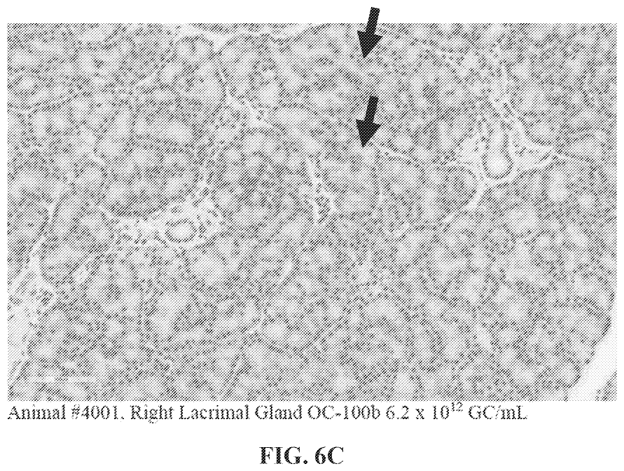

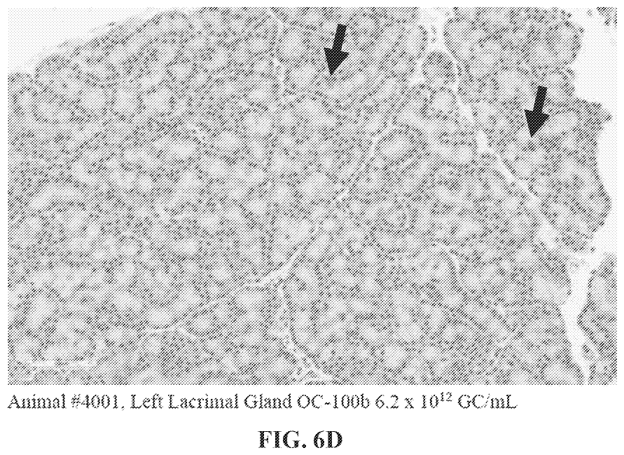

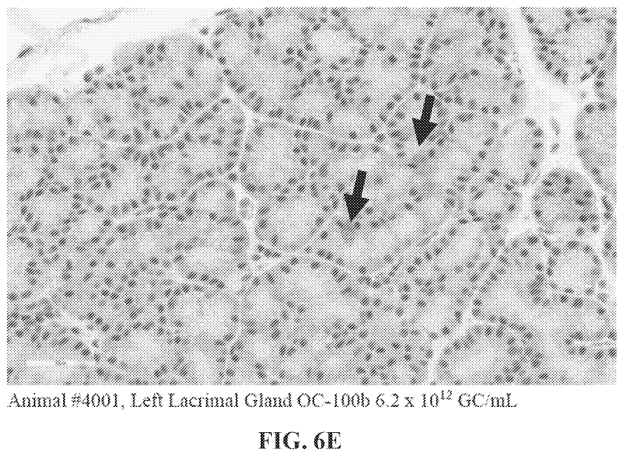

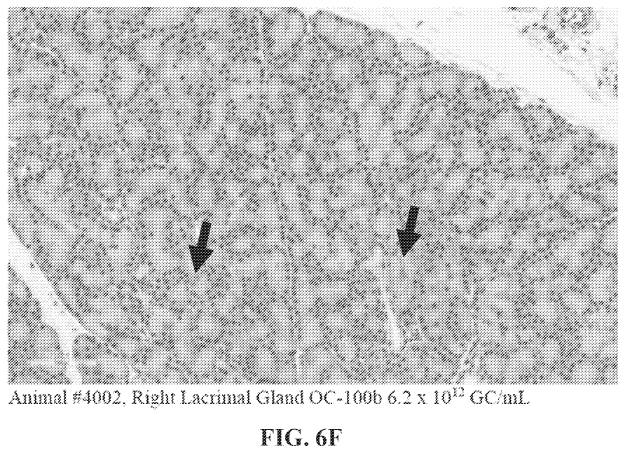

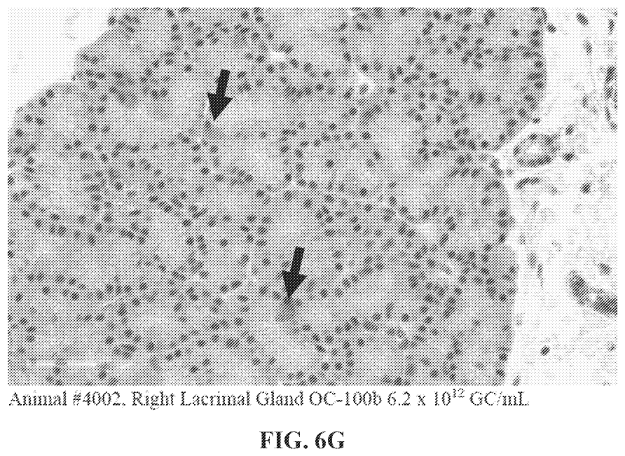

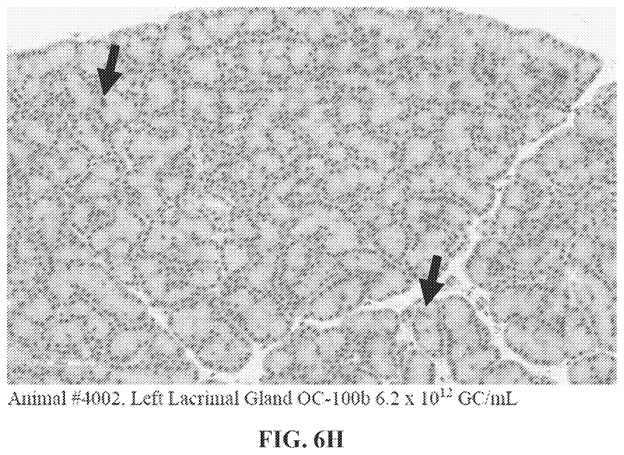

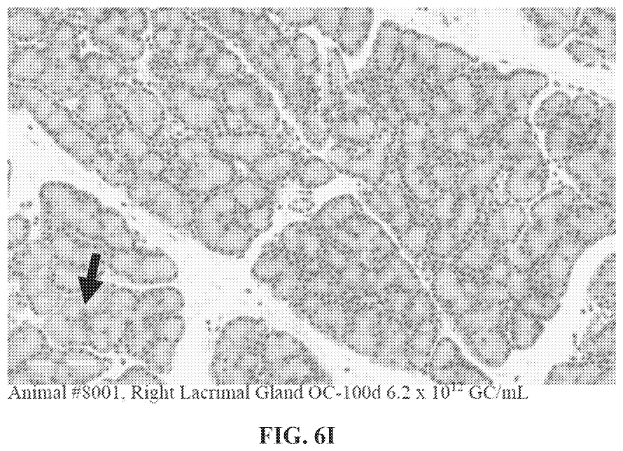

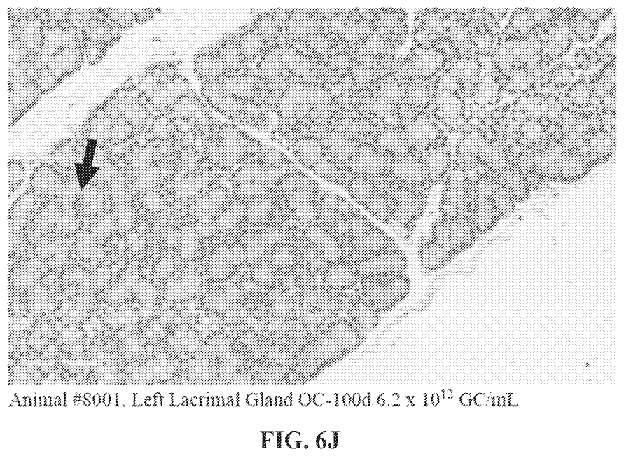

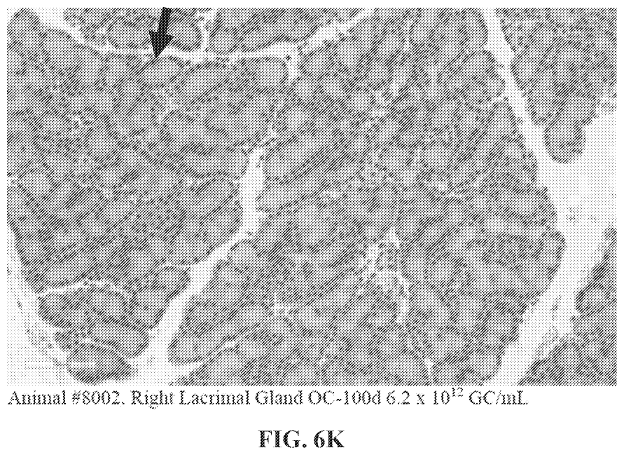

Microscopic evaluation was performed to determine the efficiency of eGFP expression in lacrimal gland tissues dosed in vivo with the rAAV compositions. Isolated positive acinar cells in the IHC samples had pink to red cytoplasmic staining indicative of GFP expression (FIGS. 6A-6K; exemplary staining indicated by black arrows). Positive eGFP expression was observed for the rAAV composition containing an AAV2 capsid protein (OC-100a) at 6.2×10¹² GC/mL (FIG. 6A), the rAAV composition containing an AAV5 capsid protein at both 1×10¹² GC/mL (FIG. 6B) and 6.2×10¹² GC/mL (FIGS. 6C-6H), and the rAAV composition containing an AAV9 capsid protein at 6.2× 10¹² GC/mL (FIGS. 6I-6K).

### Conclusion

These results from this example show that rAAV virions can be used to deliver an expression cassette to the lacrimal gland by direct injection. The results also show that rAAV virions containing capsid proteins with at least the AAV2, AAV5, or AAV9 serotypes can be used to deliver an expression cassette to cells within the lacrimal gland. Furthermore, the results demonstrate delivery of an expression cassette containing a transgene operatively linked to a CAG promoter sequence results in expression of the transgene in the cells of the lacrimal gland.

### Example 2

Expression of Neurotrophic Factors in Lacrimal Gland Delivered by rAAV via Intralacrimal Injection

This example describes a seven-day, single-dose study of varenicline administered as a single intranasal dose to Dutch-Belted rabbits after an AAV vector is administered as an intra-lacrimal gland injection. It evaluates the tolerability and effectiveness of various AAV vectors at two dose levels (1×10¹² GC/mL or 6.2×10¹² GC/mL as a 100 μL injection) administered as an intra-lacrimal gland injection followed by a single administration of varenicline nasal spray (1.2 mg/ml as a 50 μL spray) into each nostril of the subject. Specifically, this study design has two main objectives:
(1) To assess transduction of the lacrimal gland using adeno-associated virus vectors of serotype 2, 5, or 9 (AAV2, AAV5, AAV9) containing an expression cassette encoding either human nerve growth factor (hNGF) or human glial-derived neurotrophic factor (hGDNF) administered by intralacrimal injection. hNGF and hGDNF proteins are neutrophic factors that play a role in the maintenance and health of the corneal surface.
(2) To assess increasing the relative amount of protein present on the ocular surface by stimulating tear production with varenicline nasal spray.
   Varenicline is the following compound:

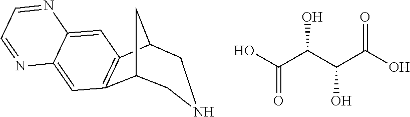 [SMILES: c1cnc2cc3c(cc2n1)C1CNCC3C1 ; O=C(O)[C@H](O)[C@@H](O)C(=O)O]

In this study an experimental viral vector or a control vector is administered to the lacrimal gland of Dutch-Belted Rabbits. The AAVx.hNGF and AAVx.hGDNF (x being 2, 5, or 9) vectors include a transgene encoding hNGF and hGDNF proteins, respectively. hNGF and hGDNF are illustrative therapeutic proteins to demonstrate feasibility for intralacrimal expression of AAV delivered transgene, whose secretion into tear film can be increased using a tear inducing agent, such as varenicline. AAVx.nNGF and AAVx.hGDNF is administered at a dose of 6.2×10¹¹ GC/mL. A vehicle control is used for comparison.

Animals are evaluated under anesthesia on Day 7 via slit lamp biomicroscopy using a blue light and appropriate filters. This evaluation is documented with slit lamp photography.

On Day 8, animals are administered a single 50 μL spray of 1.2 mg/mL varenicline nasal spray into each nostril. Immediately or within minutes after administration, animals are evaluated under anesthesia via slit lamp biomicroscopy using a blue light and appropriate filters. This evaluation is documented with slit lamp photography.

### Methods

Dose administration (route): Intralacrimal injection, 100 μL into each lacrimal gland. Dose Frequency: Single injection into each lacrimal gland. Procedure: Under anesthesia, approximately 100 μL of Reference Item 1 (PBS+0.001% F68), AAVx.hNGF (6.2×10¹¹ GC/mL), or AAVx.hNGF (6.2×10¹¹ GC/mL) is administered to each lacrimal gland under direct visualization. Lacrimal gland injection procedure is as follows: (1) a vertical incision is made underneath the right (OD) superior lid; (2) blunt dissection extends to the superior orbital rim until the lacrimal gland is visualized; (3) the lacrimal gland is injected with a 0.1 mL of Reference Item 1, AAVx.hNGF (6.2×10¹² GC/mL), or AAVx.hNGF (6.2×10¹² GC/mL) with a 1-mL sterile syringe with a 27G ½-inch sterile needle; (4) the incision is closed with sutures/cyanoacrylate glue; and (5) the procedure is repeated for the left (OS) lacrimal gland. Gene therapy administration is summarized in Table 6.

TABLE 6
Administration of rAAV

| | | | | | No. |
|---|---|---|---|---|---|
| | | Formulation | | | Animals |
| | | Concentration | | | (M/F) |
| Group | | (genome | Dose Volume | Dose | Main |
| No. | Dose | copies/volume) | (μL/animal/dose) | Frequency | Study |
| 1 | Reference | NA | 100 μL injection | once | 2/2 |
| | Item 1 | | into each gland | | |
| 2 | AAV2.hNGF | 6.2 × 10¹² GC/mL | 100 μL injection | once | 2/2 |
| | | | into each gland | | |
| 3 | AAV5.hNGF | 6.2 × 10¹² GC/mL | 100 μL injection | once | 2/2 |
| | | | into each gland | | |
| 4 | AAV9. hNGF | 6.2 × 10¹² GC/mL | 100 μL injection | once | 2/2 |
| | | | into each gland | | |
| 5 | AAV2.hGDNF | 6.2 × 10¹² GC/mL | 100 μL injection | once | 2/2 |
| | | | into each gland | | |
| 6 | AAV5.hGDNF | 6.2 × 10¹² GC/mL | 100 μL injection | once | 2/2 |
| | | | into each gland | | |
| 7 | AAV9. hGDNF | 6.2 × 10¹² GC/mL | 100 μL injection | once | 2/2 |
| | | | into each gland | | |

### Nasal Spray Tear Stimulation

Dose administration (route): Intranasal spray, both nostrils. Dose Frequency: One single administration; 50 μL spray once into each nostril; Day 8. Procedure: Animals is administered a 50 μL spray into each nostril utilizing an Aptar, Preservative Free nasal pump filled with varenicline solution 1.2 mg/ml. Termination: Day 8. Nasal spray tear stimulation is summarized in Table 7.

TABLE 7
Administration of varenicline

| | | | | | | No. |
|---|---|---|---|---|---|---|
| | | | Formulation | | | Animals |
| | | Dose | Concentration | Dose | | (M/F) |
| Group | | (mg/ | (weight/ | Volume^{a} | Dose | Main |
| No. | Test Item | animal) | volume) | (μL/animal/day) | Frequency | Study |
| 1 | Reference | 0.12 mg | NA | 100 μL | once | 2/2 |
| | Item 2 | | | | | |
| | (PBS + 0.001% | | | | | |
| | Pluronic | | | | | |
| | F-68) | | | | | |
| 2 | Varenicline | 0.12 mg | 1.2 mg/ml | 100 μL | once | 2/2 |
| 3 | Varenicline | 0.12 mg | 1.2 mg/ml | 100 μL | once | 2/2 |
| 4 | Varenicline | 0.12 mg | 1.2 mg/ml | 100 μL | once | 2/2 |
| 5 | Varenicline | 0.12 mg | 1.2 mg/ml | 100 μL | once | 2/2 |
| 6 | Varenicline | 0.12 mg | 1.2 mg/ml | 100 μL | once | 2/2 |
| 7 | Varenicline | 0.12 mg | 1.2 mg/ml | 100 μL | once | 2/2 |

^{a}The dose volume is administered to each animal, with the total volume to be administered 100 μL split equally between each nostril (50 μL per nostril per dose).

Transgene mRNA Quantification in the Lacrimal Tissue

The required section of lacrimal gland (0.005 g to ˜0.025 g) was immediately placed in RNAlater® (fully immersed) and stored at room temperature and within 8 hours was transferred to a refrigerator set to maintain a temperature of 4° C. for overnight storage. On the following day, the RNAlater® was removed and the samples were stored frozen in a freezer set to maintain a temperature −80° ° C. The samples were analyzed for hNGF expression using RT-qPCR.

The samples of (0.005 g to 0.01 g) from the harderian gland, optic nerve, trigeminal nerve, nasal cavity via epithelial cell scraping, nictitating membrane, and extraocular muscles will be collected from the right side and placed in RNAlater (fully immersed) and stored at room temperature and within 8 hours will be transferred to a refrigerator set to maintain a temperature of 4° C. for overnight storage. On the following day, the RNAlater® will be removed and the samples will be stored frozen in a freezer set to maintain a temperature −80° C. for potential vg DNA and mRNA analysis.

### Histology for Immunohistochemistry (IHC)

The appropriate section of left and right lacrimal glands were harvested for morphologic and immunohistochemical evaluation. Samples were embedded in 4% paraformaldehyde and fixed in 70% ethanol. After paraffin blocking, 5-μm thick sections were obtained and mounted on a slide; for each sample one section was stained with H&E and immunohistochemistry staining were also performed. Staining of hNGF was accomplished using an anti-hNGF antibody. Slides were observed and images captured by microscope are assessed for hNGF protein expression.

### Schirmer Test

A Schirmer tear test was performed to collect eye moisture from the animals on Days 7, 14, 21, 28, 41, and 42. Test strips were placed inside the lower eyelid for approximately 1 minute. The paper was removed and placed into separate tubes and frozen at −80° C. for subsequent analysis. Schirmer test strips were collected following intranasal administration of varenicline on Days 21, 28, and 41. Test strips were placed inside the lower eyelid for approximately 1 minute. The paper was removed and placed into separate tubes and frozen at −80° C. for subsequent analysis. Protein content absorbed onto Schirmer's test strips was analyzed for concentration of hNGF by ELISA assay.

### Results

Quantification of transgene expression in the lacrimal gland is shown in Table 8.

TABLE 8
Expression of neurotrophic factors in lacrimal gland

| | | | No. | | | mRNA | | | |
|---|---|---|---|---|---|---|---|---|---|
| | | Formulation | Animals | | No. | (ss copies/μg total RNA) | | | |
| | | Dose Level | Main | | Lacrimal | | | | |
| | | GC/gland/ | Study | | Glands | Mean | | St. Dev. | |
| GroupNo. | rAAV Vector | dose | M | F | Injected | RT | LT | RT | LT |
| 1 | Vehicle | NA | 2 | 2 | 8 | 0.0 | 0.0 | 0.0 | 0.0 |
| | Control | | | | | | | | |
| 2 | OC-101a | 6.2 × 10¹¹ | 2 | 2 | 8 | 10673.8 | 0.0 | 15505.4 | 0.0 |
| | (AAV2.hNGF) | | | | | | | | |
| 3 | OC-101b | 6.2 × 10¹¹ | 2 | 2 | 8 | 121597.6 | 156887.1 | 223042.3 | 286366.3 |
| | (AAV5.hNGF) | | | | | | | | |
| 4 | OC-101c | 6.2 × 10¹¹ | 2 | 2 | 8 | 18994.5 | 87761.3 | 31843.4 | 160533.3 |
| | (AAV9.hNGF) | | | | | | | | |
| 5 | OC-102a | 6.2 × 10¹¹ | 2 | 2 | 8 | 3674.2 | 1322.3 | 4906.5 | 1750.4 |
| | (AAV2.hGDNF) | | | | | | | | |
| 6 | OC-102b | 6.2 × 10¹¹ | 2 | 2 | 8 | 8817.6 | 124730.8 | 14948.1 | 249461.5 |
| | (AAV5.hGDNF) | | | | | | | | |
| 7 | OC-102c | 6.2 × 10¹¹ | 2 | 2 | 8 | 6271.2 | 3520.7 | 4671.0 | 3815.8 |
| | (AAV9.hGDNF) | | | | | | | | |

Tissues from all vehicle control animals tested negative for bGH gene expression, demonstrating that cross contamination was well controlled from animal dosing, sample collection, RNA extraction and RT-qPCR analysis. rAAV vector-derived hGDNF mRNAs were detected in all injected lacrimal gland tissues. For Groups 3 to 6, some injected lacrimal gland tissues had hNGF or hGDNF mRNAs from 1151 to 585,401 ss copies per ug of total RNA, while some others had no detectable vector-derived mRNA.

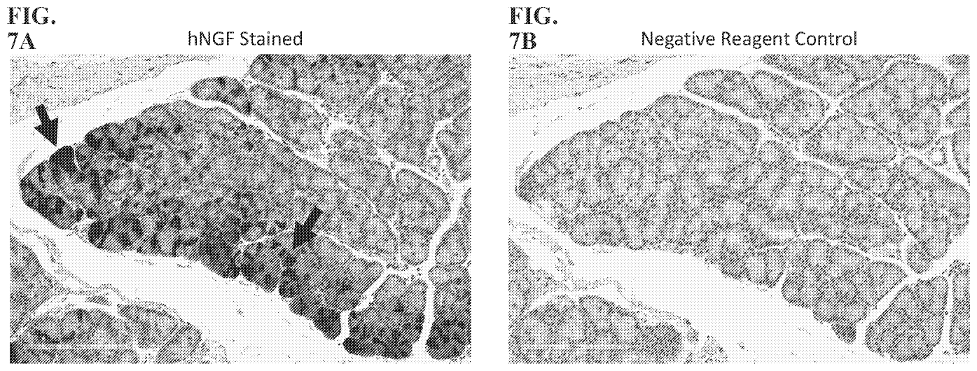

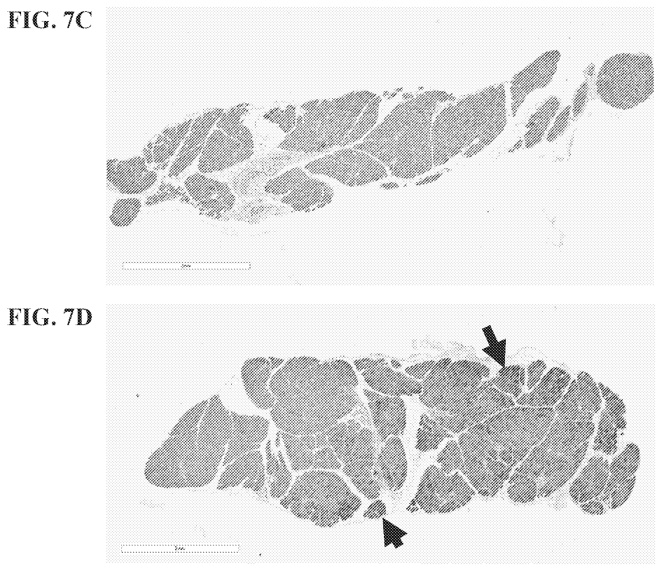

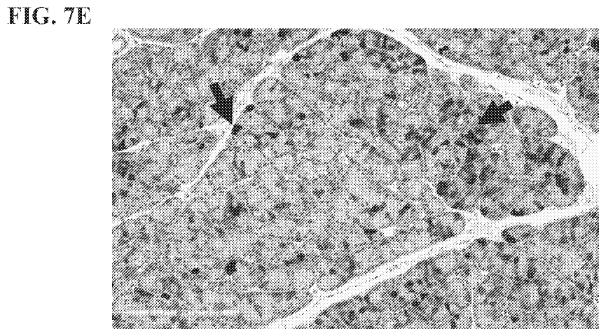

Lacrimal al tissue was further assessed for hNGF expression using immunohistochemistry (FIGS. 7A-7E). Following intralacrimal injection, animals were euthanized and sections of the injected lacrimal glands were prepared for immunohistochemistry (IHC). Lacrimal gland IHC samples were stained for hNGF and subject to microscopic evaluation. Microscopic evaluation was performed to determine the efficiency of hNGF expression in lacrimal gland tissues dosed in vivo with the rAAV compositions. FIG. 7A and FIGS. 7D-7E show illustrative examples of IHC staining in the lacrimal gland of rabbits injected with rAAV with an AAV9 serotype (exemplary staining shown by black arrows). The hNGF stained tissue shows strong expression of the hNGF transgene. The result is confirmed by comparing with the negative control sample (FIG. 7B and FIG. 7C), which shows little staining and thus rules out staining due to background. These results indicate intralacrimal injection of rAAV can be used to deliver and express a neurotrophic factor transgene, e.g. hNGF, in the lacrimal tissue.

A Schirmer tear test was performed to collect eye moisture from the animals on Days 7 and 14 following administration of rAAV. Test strips were placed inside the lower eyelid for approximately 1 minute. The paper was removed and placed into separate tubes and frozen at −80° C. for subsequent analysis. Test strips were placed inside the lower eyelid for approximately 1 minute. The paper was removed and placed into separate tubes and frozen at −80° C. for subsequent analysis. Protein content absorbed onto Schirmer's test strips was analyzed for concentration of hNGF by ELISA assay.

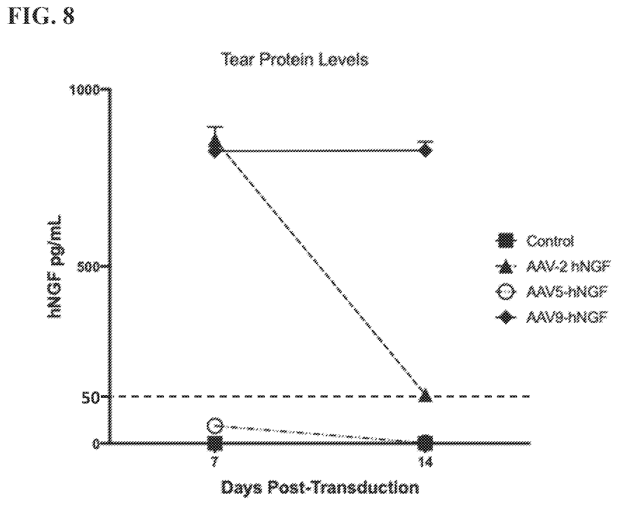

Schirmer's test strips were analyzed for concentration of hNGF in the collected tear film (FIG. 8). Normal NGF concentration in tears is expected to be about 50 pg/mL. Following intralacrimal administration of rAAV9.hNGF or rAAV2.hNGF, the hNGF concentration was over 100-fold greater than the normal concentration. On day 14, the concentration of hNGF in rabbits dosed with rAAV9.hNGF remained over 100-fold greater than the normal concentration, and the hNGF concentration in rabbits dosed with rAAV2.hNGF dropped, but remained well above the no rAAV control group. In rabbits dosed with rAAV5.hNGF showed an in increased of hNGF concentration at day 7, and dropped back to control levels by day 14.

In summary, the results show that neurotrophic factors, such as hNGF and hGDNF, are expressed as transgenes when delivered via intralacrimal injection with rAAV, including AAV2, AAV5, and AAV9 serotypes, with AAV9 serotype showing the best results.

### Example 3

### Expression of EGFP Transgene in Porcine Lacrimal Gland Delivered by rAAV via Intralacrimal Injection

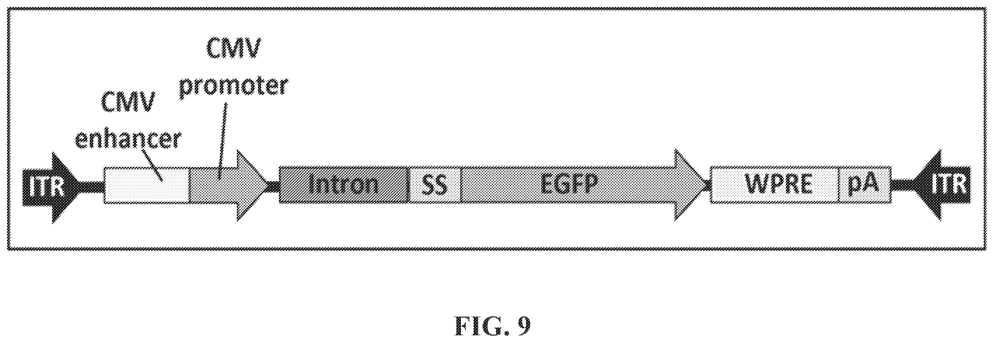

The study objective was to assess if the lacrimal gland is able to be leveraged as a method to modify or enrich the tear film with a protein of interest in pigs. Subsequently, in vivo study was performed to test if EGFP could be produced in the acinar cells of the lacrimal gland and then secreted into the tear film after delivery of an adenoviral vector consisting of a plasmid encoding eGFP. To get cDNA encoding EGFP into acinar cells, our approach was to inject the lacrimal gland with an adeno-associated virus (AAV) which contained cDNA encoding for secreted EGFP (secEGFP). To create each AAV of 2 different serotypes (2 and 9) for secEGFP, an AAV transfer plasmid was generated which contained between the inverted terminal repeats (ITRs) the essential elements for secEGFP expression. The DNA sequence between the ITRs was packaged into the AAV (FIG. 9) that was manufactured.

Eleven serotypes of AAV have been identified with the best characterized being AAV2. AAV pseudotyping is the mixing of a capsid and genome from different serotypes to improve transduction efficiency and these serotypes are denoted with a slash. For example, AAV2/5 indicates a virus containing serotype 2 packaged in the capsid from serotype 5. AAV5 and AAV9 have been reported to be capable of delivering a luciferase reporter gene to the lacrimal gland of mice (Rocha et al., Transduction, Tropism, and Biodistribution of AAV Vectors in the Lacrimal Gland, 2011; 52 (13):9567-9572). Mouse lacrimal gland transduction with GFP as well as mouse nerve growth factor (mNGF) has been observed for pseudotypes AAV2/5 and AAV2/9 and reported recently on a pre-print server site (Gautier et al., AAV2/9-mediated gene transfer into murine lacrimal gland leads to a long-term targeted tear film modification; bioRxiv, 2022).

### Design Analysis and Methodology

Research grade AAVs for secreted EGFP (serotypes 2 and 9) were synthesized at Sirion and were provided at a stock concentration of 5×10¹². The AAVs were in vitro tested by CJ Solutions using HEK 293T cells and ELISA to ensure that the manufactured AAVs would transduce cells. At Texas A&M, eight domestic pigs received a one-time intralacrimal gland injection of EGFP with the right (OD; oculus dexter) gland receiving a low dose and the left (OS; oculus sinister) gland receiving a high dose. Six weeks after the first injection, a second injection with AAV2 and AAV9 high doses were performed. The study assessed EGFP expression at Day 35. Following tear EGFP level confirmation, the study was terminated 8 weeks after the second injection to assess the presence of EGFP in the lacrimal glands and assess any potential inflammation or gland abnormalities. (Tables 9 & 10). In the study, nasal spray dosing was administered between weeks 3 to 4 (Table 11).

TABLE 9
Study plan for in vivo study of AAV2-secEGFP and AAV9-secEGFP in domestic pigs.

| | | |
|---|---|---|
| Title | | Fifteen week porcine study of AAV2-secEGFP and |
| | | AAV9-secEGFP following repeat intralacrimal gland |
| | | injection and OC-01 nasal spray administration. |
| GLP Status | | No |
| Objective(s) | | Fifteen week in vivo study to assess lacrimal gland |
| | | transduction following two intralacrimal gland injections |
| | | given 6 weeks apart. An initial of low-dose (1 × 10⁹ vg)- |
| | | or high-dose (1 × 10¹¹ vg) of AAV2-secEGFP or AAV9- |
| | | secEGFP was administered at Day 0 to transduce the |
| | | porcine lacrimal gland followed by a high dose (1 × 10¹¹ vg) |
| | | at week 6 (Day 47). The results are needed to: 1) confirm |
| | | expression and secretion of EGFP; 2) determine if EGFP is |
| | | detectable up to 8 weeks post-injection; and 3) assess any |
| | | AAV related safety signals following repeat |
| | | administration. |
| Study Animals | Breed | Domestic pig |
| | Species | Sus scrofa domesticus |
| | Age | 8-10 weeks |
| | Body | ~18-23 kg |
| | Weight | |
| | Sex—(#M/#F) | 8 males |
| | Total | 8 for study (+naïve) |

| | |
|---|---|
| Test article(s) | First Injection—AAV2-secEGFP injected at a 1.0 × 10⁹ vg |
| | dose (OD) or 1.0 × 10¹¹ vg dose (OS) into the lacrimal |
| | gland |
| | AAV9-secEGFP injected at a 1.0 × 10⁹ vg dose (OD) or |
| | 1.0 × 10¹¹ vg dose (OS) into the lacrimal gland |
| | Second injection: AAV2-secEGFP and AAV9-secEGFP at |
| | a 1 × 10¹¹ vg dose |
| Route of Administration | Intralacrimal gland injection for AAV and intranasal dose |
| | OC-01 |
| Study Groups and Dose | See Table Below |
| Levels | |
| Dosing Regimen | 1. First injection into each lacrimal gland; |
| | OD: single injection of 40 μL low dose |
| | OS: single injection of 40 μL high dose |
| | 2. Nasal spray given twice daily from day 21 to day 28 |
| | 3. Second injection into each lacrimal gland |
| | 4. |
| Randomization | No |
| Planned Sacrifice Timepoint | Day 103 |

TABLE 10
Injected dose and volume of AAV.

| | | | Dose | | | Number |
|---|---|---|---|---|---|---|
| | | | number | | | of |
| | | | and | | | Animals |
| | Dose Level | Injection | volume | Route | Route | (gender) |
| Group | (vg) | Time | (μL) | (Right Gland) | (Left Gland) | M |
| 1 | OD: low dose | Day 0 | OD: | Intralacrimal | Intralacrimal | 4 |
| | 1.0 × 10⁹ | | 40 μL | AAV2- | AAV2- | |
| | OS: high dose | | single | secEGFP | secEGFP | |
| | 1.0 × 10¹¹ | | injection | OD: 40 μL | OS: 40 μL | |
| | | | OS: | single injection | single injection | |
| | | | 40 μL | low dose | high dose | |
| | | | single | | | |
| | | | injection | | | |
| 2 | OD: low dose | Day 0 | OD: | Intralacrimal | Intralacrimal | 4 |
| | 1.0 × 10⁹ | | 40 μL | AAV9- | AAV9- | |
| | OS: high dose | | single | secEGFP | secEGFP | |
| | 1.0 × 10¹¹ | | injection | OD: 40 μL | OS: 40 μL | |
| | | | OS: | single injection | single injection | |
| | | | 40 μL | low dose | high dose | |
| | | | single | | | |
| | | | injection | | | |
| 1 | OD: high dose | Day 47 | OD: | Intralacrimal | Intralacrimal | 4 |
| | 1 × 10¹¹ | | 40 μL | AAV9- | AAV9- | |
| | OS: high dose | | single | secEGFP | secEGFP | |
| | 1.0 × 10¹¹ | | injection | OD: 40 μL | OS: 40 μL | |
| | | | OS: | single injection | single injection | |
| | | | 40 μL | | | |
| | | | single | | | |
| | | | injection | | | |
| 2 | OD: high dose | Day 47 | OD: | Intralacrimal | Intralacrimal | 4 |
| | 1 × 10¹¹ | | 40 μL | AAV2- | AAV2- | |
| | OS: high dose | | single | secEGFP | secEGFP | |
| | 1.0 × 10¹¹ | | injection | OD: 40 μL | OS: 40 μL | |
| | | | OS: | single injection | single injection | |
| | | | 40 μL | | | |
| | | | single | | | |
| | | | injection | | | |

vg = viral genomes

TABLE 11
OC-01 nasal spray dosing was from day 21 to day 28.

| | | | | Number |
|---|---|---|---|---|
| | | | | of |
| | | | | Animals |
| | OC-01 | | | (gender) |
| Group | Dose Level | Dose | Dose number | Male |
| 1-Bilateral | 0.05 mL of | BID | Twice daily from | 4 |
| nares dose | 30 mcg | | day 21-28 | |
| 2-Bilateral | 0.05 mL of | BID | Twice daily | 4 |
| nares dose | 30 mcg | | from day 21-28 | |

mcg = micrograms

Following the second AAV-secEGFP injection, tears were collected from each eye via Schirmer strips on day 82. Tears were collected by placing a Schirmer's Tear Test strip in the lower conjunctival cul-de-sac and leaving in place for 2 minutes. Tear protein was extracted from the Schirmer's Tear Test strip and mesoscale discovery (MSD) analysis was conducted to detect the presence of EGFP protein in the tears.

Lacrimal gland was collected for ocular histopathology on Day 103 and samples were sent to Zyagen, Inc. (San Diego, CA) for EGFP immunohistochemistry (IHC).

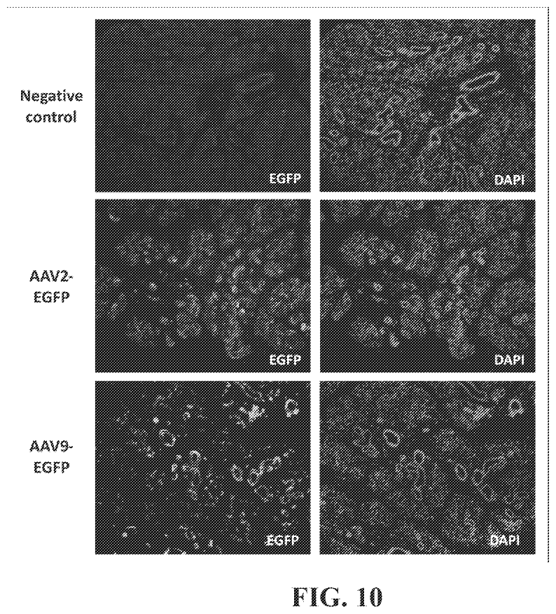

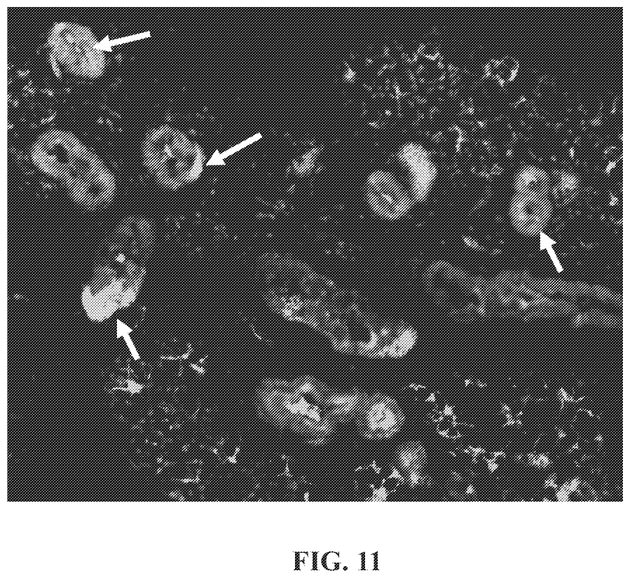

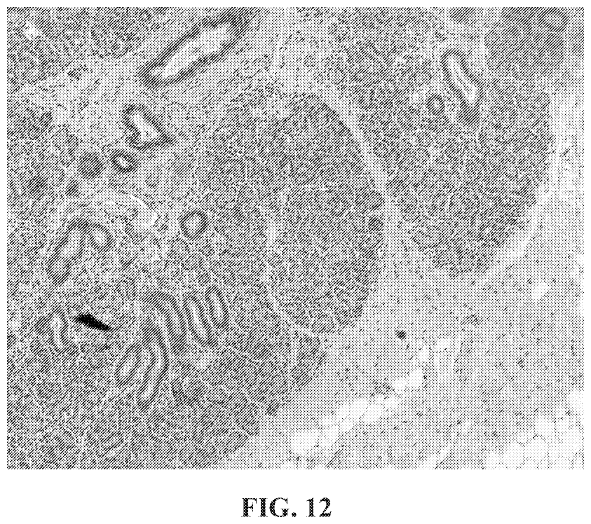

ELISA results showed that the AAV serotypes that were manufactured could transduce HEK 293T cells and produced secreted EGFP in vitro. EGFP expression in the tear samples was confirmed by MSD analysis 82 days after AAV transduction with eGFP some levels >400 pg/mL as well as by IHC (FIG. 10). IHC indicated that EGFP expression was within the acinar cells with greater acinar cell infectivity observed for AAV2 compared to AAV9. Additionally, transduction of ductile epithelial cells was observed for AAV9 injected lacrimal glands (FIG. 11). Hematoxylin and eosin staining of pig lacrimal gland after repeat AAV injections did not show any inflammatory infiltrate, atrophy or edema (FIG. 12).

Porcine lacrimal gland that is injected with either AAV2-secEGFP or AAV9-secEGFP expressed the EGFP transgene product in acinar cells as well as ductile epithelial cells. The EGFP that was expressed in the lacrimal gland was found to be secreted into the tear film. Additionally, no safety signals or inflammatory infiltrates were observed in any animals after repeat injections of AAV2 or AAV9 regardless if they initially received a low or high dose of AAV during the first injection. The results of this study demonstrate that the acinar cells of the lacrimal gland are a target for a gene therapy approach to modify and/or enrich the tear film.

### Example 4

### Detection of Human Nerve Growth Factor Beta Protein in Porcine Tears Following Transduction

Tears were collected from twelve pigs (Sus scrofa domesticus) seven (7), fourteen (14), twenty-one (21), twenty-eight (28), and thirty-five (35) days following transduction of lacrimal gland (via intralacrimal injection) using a single dose (2ell vg/mL) of an adeno-associated viral vector (AAV) encoding human nerve growth factor (AAV-hNGFβ) utilizing adeno-associated virus serotypes 2 (n=4), 5 (n=4), and 9 (n=4) vectors. Collection of tear samples was performed on Day 7, 14, 21, 28, and 35 utilizing Schirmer Tear Test strips placed under the lower eyelid of both the right (OD) and left (OS) eyes individually for approximately 120 seconds in anesthetized animals. On Days 22-27, 0.03 mg varenicline solution was administered to each nostril twice a day (BID). For the Day 28 tear collection, 0.03 mg of varenicline solution was administered to each nostril via a nasal spray pump 2 minutes prior to tear collection. On all collection days, the Schirmer Tear Test strip was cut just above the fluid migration line and then the fluid-saturated portion of the test strip was immediately placed into a microcentrifuge on ice. Collected samples were stored at −20 Celsius before being shipped to a testing facility on dry ice.

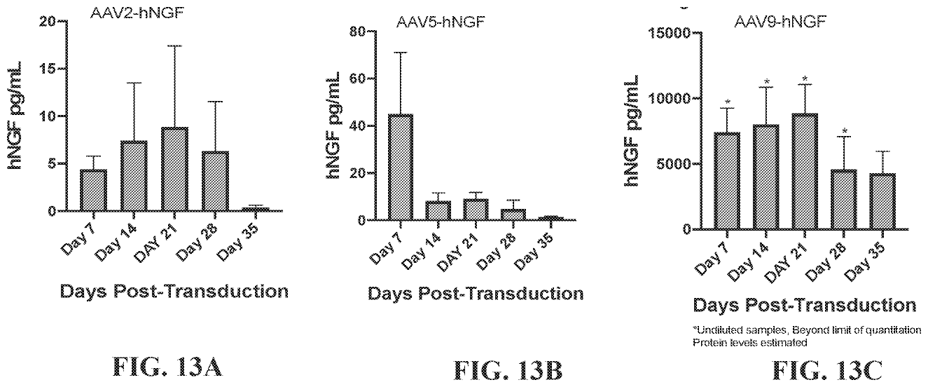

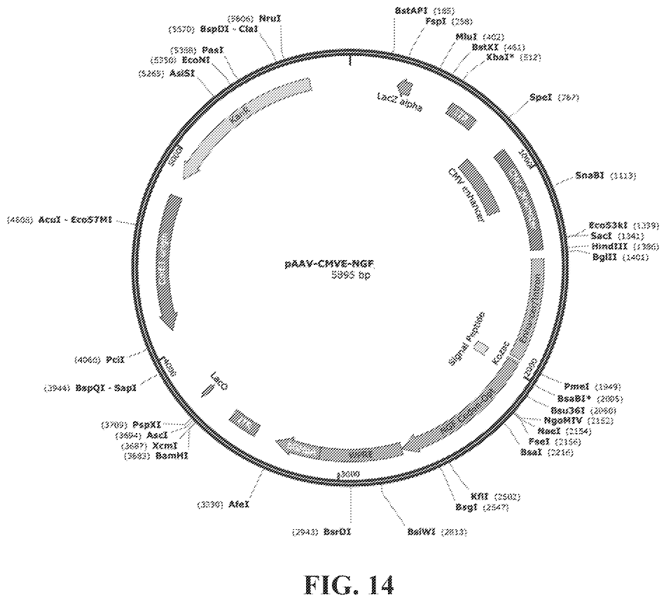

Protein was extracted from the Schirmer Tear Test strips of the left eye and a meso-scale discovery assay detecting human nerve growth factor protein (hNGFβ) was conducted on each sample. The minimum and maximum standard curve values of the MSD assay for human NGFβ were 0.104 to 427 pg/mL, respectively. Transduction of (comprising the expression cassette of SEQ ID NO: 25; depicted in FIG. 14) generated expression of hNFGβ in tears (FIGS. 13A-13C, respectively). The AAV9-hNGFß transduction generated such high expression that the sample had to be diluted 16-128× for detection (Table 12). The average calculated tear film level of hNGFB from AAV9-hNGFB transduced pigs derived from the dilution study is 5,612.84 pg/mL. It should be noted that hNGFβ was not detected in non-transduced porcine tear film based upon hNGF MSD data collected from previous studies utilizing AAV vectors encoding different proteins. Therefore, any hNGFβ protein detected from extracts of the Schirmer strip tear samples was easily identified during the MSD assay and attributed to transduction of the lacrimal gland by AAV-hNGFβ constructs. The samples collected on Day 21 for AAV2-hNGFβ (FIG. 13A) includes an outlier (p<0.05 as determined by Grubb's test) which was omitted from the analysis. No significant decrease was observed between Days 7-28 for AAV9-hNGFβ as shown in FIG. 13C). Additionally, hNGF protein levels were compared between samples from animals administered AAV9-hNGFβ collected on Day 21 (no nasal spray), Day 28 (nasal spray administration), and Day 35 (no nasal spray). Administration of nasal spray increased tear hNGFβ protein concentration when administered the nasal spray compared to AAV administration alone (data not shown).

Together, this data demonstrates transduction of the porcine lacrimal gland with an adenoviral-associated vector encoding hNGFβ resulted in expression, secretion, and transport of hNGFB to the tear film in detectable quantities. Expression was detectable within 7 days of lacrimal gland transduction with significant variability in expression levels dependent upon the AAV serotype utilized. AAV9 demonstrated the highest level of expression within 7 days and AAV2 showed the lowest level, albeit still within the range of the analytical assay utilized for detection of human nerve growth factor beta protein. AAV2 protein levels in tears is steadily increased out to 28 days, while AAV5 decreased after the first 7 days and appeared to have reached a steady-state at Days 14 through28 with further reduction at day 35. AAV9 transduced animals had a significant level of hNGF detected in the tear film at Day 7 that remained constant out to Day 35.

TABLE 12
Serial dilution of AAV9-hNGFβ tear sample

| Dilution of AAV9- | hNGFβ Concentration | |
|---|---|---|
| hNGF Tears | (pg/mL) | Range of Detection |
| 1:2 | 8,729.83 | Above Upper Assay Limit |
| 1:4 | 10,001.39 | Above Upper Assay Limit |
| 1:8 | 7,613.18 | Above Upper Assay Limit |
| 1:16 | 6,439.27 | Within Range of Detection |
| 1:32 | 5,499.16 | Within Range of Detection |
| 1:64 | 4,763.83 | Within Range of Detection |
| 1:128 | 5,749.08 | Within Range of Detection |

While embodiments of the present invention have been shown and described herein, those skilled in the art will understand that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of treating an ocular condition in a subject in need thereof, the method comprising administering a recombinant adeno-associated virus (“rAAV”) virion, the rAAV virion comprising an adeno-associated virus (“AAV”) capsid and an expression cassette comprising a polynucleotide encoding a neurotrophic factor operatively linked to a promoter, to at least one eye of the subject or to at least one lacrimal gland of the eye of the subject.

2. The method of claim 1, wherein the rAAV virion is administered to a lacrimal gland of the subject.

3. The method of claim 2, wherein the lacrimal gland is
(a) the main lacrimal gland or any one of the Wolfring's glands or the Krause's glands of the subject; and/or
(b) the main lacrimal gland.

4. (canceled)

5. The method of claim 2, wherein cells within the lacrimal gland are transduced by the rAAV virion.

6. The method of claim 5, wherein the transduced cells within the lacrimal gland express an effective amount of the neurotrophic factor into the tear film and optionally onto the ocular surface of the subject.

7. The method of claim 1, wherein the neurotrophic factor is a Nerve Growth Factor (“NGF”) protein.

8. The method of claim 7, wherein
(a) the polynucleotide encoding the NGF protein comprises a sequence that shares at least 95% identity to SEQ ID NO: 2;
(b) the NGF protein comprises a sequence that shares at least 95% identity to SEQ ID NO: 1;
(c) the NGF protein comprises SEQ ID NO: 1; and/or
(d) the polynucleotide encoding the NGF protein comprises SEQ ID NO: 2.

9-11. (canceled)

12. The method of claim 1, wherein the neurotrophic factor is a Glial Derived Neurotrophic Factor (“GDNF”) protein.

13. The method of claim 12, wherein
(a) the polynucleotide encoding the GDNF protein comprises a sequence that shares at least 95% identity to SEQ ID NO: 4;
(b) the GDNF protein comprises a sequence that shares at least 95% identity to SEQ ID NO: 3;
(c) the GDNF protein comprises SEQ ID NO: 3; and/or
(d) the polynucleotide encoding the GDNF protein comprises SEQ ID NO: 4.

14-16. (canceled)

17. The method of claim 1, wherein the AAV capsid shares at least 95%, 98%, or 100% identity to SEQ ID NO: 6, SEQ ID NO: 8, SEQ ID NO: 10, SEQ ID NO: 12, or SEQ ID NO: 14.

18. The method of claim 1, wherein the promoter is a CAG promoter.

19. The method of claim 1, wherein the ocular condition is a chemical burn of the ocular surface, corneal wound, corneal ulcer, persistent epithelial defect, dry eye disease, neurotrophic keratitis, herpes simplex viral infection of the trigeminal nerve and/or the eye, varicella zoster virus infection of the trigeminal nerve and/or the eye, or diabetic complications of the corneal nerves.

20. The method of claim 1, wherein one or more symptoms of the ocular condition are reduced compared to the symptoms of the ocular condition before administration of the rAAV virion.

21. The method of claim 1, wherein one or more symptoms of the ocular condition are reduced compared to the symptoms of the ocular condition in an untreated control subject.

22. The method of claim 1, wherein one or more symptoms of the ocular condition are reduced compared to the symptoms of the ocular condition in a contralateral eye.

23. A recombinant adeno-associated virus (“rAAV”) virion, comprising an adeno-associated virus (“AAV”) capsid and an expression cassette, wherein the expression cassette comprises a polynucleotide encoding a neurotrophic factor operatively linked to a promoter.

24-38. (canceled)

39. A recombinant adeno-associated virus (“rAAV”) virion comprising an expression cassette comprising a polynucleotide sharing at least 90% sequence identity with SEQ ID NO: 25.

40. (canceled)

41. (canceled)

42. A composition comprising a recombinant adeno-associated virus (“rAAV”) virion, wherein the rAAV virion comprises:
an adeno-associated virus (“AAV”) capsid and an expression cassette, wherein the expression cassette comprises
(a) a polynucleotide sharing at least 95% identity to SEQ ID NO: 2 or SEQ ID NO: 17, and wherein the polynucleotide is linked to a promoter; or
(b) a polynucleotide sharing at least 95% identity to SEQ ID NO: 4 or SEQ ID NO: 18, and wherein the polynucleotide is linked to a promoter.

43. (canceled)

44. The composition of claim 42, wherein the AAV capsid comprises an AAV2, AAV5, AAV8, or AAV9 capsid.

45-49. (canceled)

50. A composition comprising a recombinant adeno-associated virus (“rAAV”) virion, wherein the rAAV virion comprises:
(a) an adeno-associated virus (“AAV”) capsid comprising an AAV2, AAV5 or AAV9 capsid; and
(b) an expression cassette, wherein the expression cassette comprises a polynucleotide sharing at least 90% identity to SEQ ID NO: 25.

51-75. (canceled)
